(19) United States
(12) Patent Application Publication
(10) Pub. No.: US 2022/0281966 A1
(43) Pub. Date: Sep. 8, 2022

(54) ANTI-IL13 ANTIGEN BINDING PROTEINS

(71) Applicant: AMGEN INC., Thousand Oaks, CA (US)

(72) Inventors: Agnieszka KIELCZEWSKA, Vancouver (CA); Ian Nevin FOLTZ, Burnaby (CA); Palaniswami RATHANASWAMI, Vancouver (CA); Maria Sheena AMADOR, Surrey (CA); Bram ESTES, Camarillo, CA (US); Igor D'ANGELO, Newbury Park, CA (US)

(73) Assignee: AMGEN INC., Thousand Oaks, CA (US)

(21) Appl. No.: 17/630,437

(22) PCT Filed: Jul. 24, 2020
(86) PCT No.: PCT/US2020/043607

§ 371 Date: Jan. 26, 2022

Related U.S. Application Data
(60) Provisional application No. 62/879,335, filed on Jul. 26, 2019.

(51) Int. Cl. C07K 16/24; A61P 11/00; A61P 11/08; A61P 11/06; A61P 17/00

(52) U.S. Cl. CPC C07K 16/244; A61P 11/00; A61P 11/08; A61P 11/06; A61P 17/00; C07K 2317/565; C07K 2317/56; C07K 2317/92; C07K 2317/94

(57) ABSTRACT

The present invention is related to antibodies directed to IL-13 and uses of such antibodies. For example, in accordance with the present invention, there are provided human monoclonal antibodies directed to IL-13. Isolated polynucleotide sequences encoding, and amino acid sequences comprising, heavy and light chain immunoglobulin molecules, particularly sequences corresponding to contiguous heavy and light chain sequences spanning the framework regions (FR's) and/or complementarity determining regions (CDR's), are provided. Additionally, methods of using these antibodies to treat patients are also provided.

### CROSS REFERENCE TO RELATED APPLICATIONS

This application claims priority to and the benefit of U.S. Provisional Application No. 62/879,335 filed Jul. 26, 2019, which is incorporated by reference herein in its entirety.

### REFERENCE TO THE SEQUENCE LISTING

This application contains a Sequence Listing in computer-readable form. The Sequence Listing is provided as a text file entitled A-2421-WO-PCT_SeqList_ST25.txt, created Jul. 24, 2020, which is 205,493 bytes in size. The information in the electronic format of the Sequence Listing is incorporated herein by reference in its entirety.

### FIELD OF THE INVENTION

The present invention relates to the field of biopharmaceuticals. In particular, the invention relates to antibodies that specifically bind to human IL-13 antibodies and IL-13 binding fragments and derivatives thereof. The invention also relates to pharmaceutical compositions comprising the anti-IL-13 for treating inflammatory diseases as well as methods of making such antibodies.

### BACKGROUND OF THE INVENTION

IL-13 is a cytokine that was first recognized for its effects on B cells and monocytes, where it up-regulates class II expression, promotes IgE class switching and inhibits inflammatory cytokine production. The IL-13 receptor shares the IL-4 receptor alpha chain with the IL-4 receptor. As a result, IL-13 has many similar biological activities to IL-4.

IL-13 inhibits proinflammatory cytokine release and has an anti-inflammatory activity in vivo. IL-13 plays a role in IgE mediated allergic responses and is the central mediator of allergic asthma (Wills-Karp M., Curr. Opin. Pulm. Med., 2003; 9:21-27). In the lung it regulates eosinophilic inflammation, mucus secretion, and airway hyperresponsiveness. In addition to asthma, IL-13 is implicated in the pathogenesis of a large number of diseases (Wynn T A. Annu. Rev. Immunol. 2003. 21:425-456).

The human antibody Ab731 binds to human IL-13 with high affinity. However, the antibody binds to cynomolgus monkey (Macaca fascicularis, also referred to as “cyno” IL-13 (“cyIL-13”) with a relatively low affinity. Because cynomolgus monkeys are commonly used to assess preclinical safety of antibodies, it would be desirable to have an anti-human IL-13 antibody that also bound cynomologus IL-13 at a high affinity.

### SUMMARY OF THE INVENTION

Therapeutic antibodies are desired to display high affinity binding to both the human and cynomolgus monkey orthologue of a therapeutic target. The affinity gap is required to be within a 10-fold affinity window to enable toxicological studies. AMGN12 antibody, derived from an in vivo immunization on the XenoMouse®, demonstrated many favorable properties, including single digit pM affinity to the human orthologue of the target protein. However, that antibody displayed 200 fold weaker binding to the orthologue present in the cynomolgus monkey. The goal of this work was to “close” the affinity gap without compromising binding affinity to the human target, and lead to identification of variants with an over 100 fold affinity improvement to the cynomolgus orthologue as well as a 10 fold potency improvement in biological assays for function. A high resolution crystal structure of the final variant antibody complexed with the target was resolved and illustrated that the mutations leading to affinity improvements obtained by the HuTARG platform were that would be difficult to a priori design in silico.

The present invention relates to the field of biopharmaceuticals. In particular, the invention relates to antibodies that specifically bind to human and cyno IL-13 antibodies and IL-13 binding fragments and derivatives thereof. The invention also relates to pharmaceutical compositions comprising the anti-IL-13 for treating inflammatory diseases as well as methods of making such antibodies.

In embodiments, the anti-IL-13 antibodies, antigen (IL-13) binding fragments and derivatives (collectively referred to as “antigen binding proteins”) relate to the field of biopharmaceuticals. The invention relates to anti-IL-13 antibodies and other IL-13 binding proteins that specifically bind to human IL-13. The invention also relates to pharmaceutical compositions comprising the anti-IL-13 antigen binding proteins for treating inflammatory diseases as well as methods of making such antibodies.

In an embodiment of the invention, a small amino acid sequence change to the CDRs of Ab731 increase binding to cynomologus monkey IL-13.

The invention contains the following embodiments:
1. An antigen binding protein that specifically binds to human IL-13 comprising a light chain immunoglobulin variable region (VL1) and a heavy chain immunoglobulin variable region (VH),

wherein VL1 comprises (i) a CDRL1 comprising an amino acid sequence of SEQ ID NO: 11; (ii) a CDRL2 comprising an amino acid sequence of SEQ ID NO: 12, and (iii) a CDRL3 comprising an amino acid of SEQ ID NO: 13;

and VH1 comprising an amino acid sequence of (i) comprising an amino acid sequence of SEQ ID NO: 8 (ii) a CDRH2 comprising an amino acid sequence of SEQ ID NO: 9, and (iii) a CDRH3 comprising an amino acid sequence of SEQ ID NO: 10.

2. An antigen binding protein that specifically binds to human IL-13 comprising a light chain immunoglobulin variable region (VL1) and a heavy chain immunoglobulin variable region (VH),

wherein VL comprises the CDRs of the antibody expressed by cell 623,

and VH comprises the CDRS pf the antibody expressed by cell 623.

3. The antigen binding proteins of embodiments 1 further comprising the framework regions as the antibody expressed by cell 623.
4. The antigen binding protein of embodiments 1-3 wherein the antigen binding protein is an antibody.
5. The antigen binding protein of embodiments 1-3 wherein the antigen binding protein is an antibody fragment.
6. The antigen binding protein of embodiments 1-3 wherein the antigen binding protein is an antibody derivative comprising a bispecific antibody, a fusion protein.
7. The antigen binding protein of embodiments 1-6 wherein the antigen binding protein has human sequences.
8. The antigen binding protein of embodiments 1-6 wherein the antigen binding is a monoclonal antibody.
9. A human antibody that binds to IL-13, wherein the human antibody binds to IL-13 with a K_{D} of between 2 cM to 50 pM.
10. A human antibody that binds to IL-13, wherein the human antibody binds to IL-13 with a K_{D} of 2 cM to 40pM.
11. A human antibody or antigen binding fragment thereof that binds to human IL-13 selected from the group wherein the amino acid sequences comprise

(a) an antibody variable light chain amino acid sequence comprising LCDR1 of SEQ ID NO:11, a LCDR2 of SEQ ID NO:12, and a LCDR3 of SEQ ID NO: 13; and an variable antibody heavy chain amino acid sequence comprising HCDR1 of SEQ ID NO: 8, a HCDR2 of SEQ ID NO: 106 and a HCDR3 of SEQ ID NO:10;

(b) an antibody variable light chain amino acid sequence comprising LCDR1 of SEQ ID NO:11, a LCDR2 of SEQ ID NO: 12, and a LCDR3 of SEQ ID NO: 13; and an variable antibody heavy chain amino acid sequence comprising HCDR1 of SEQ ID NO: 8, a HCDR2 of SEQ ID NO: 83; and a HCDR3 of SEQ ID NO: 10.

(c) an antibody variable light chain amino acid sequence comprising LCDR1 of SEQ ID NO:11, a LCDR2 of SEQ ID NO:12, and a LCDR3 of SEQ ID NO: 13; and an variable antibody heavy chain amino acid sequence comprising HCDR1 of SEQ ID NO: 8, a HCDR2 of SEQ ID NO: 83, and a HCDR3 of SEQ ID NO:10;

(d) an antibody variable light chain amino acid sequence comprising LCDR1 of SEQ ID NO: 74, a LCDR2 of SEQ ID NO: 12, and a LCDR3 of SEQ ID NO: 76; and an variable antibody heavy chain amino acid sequence comprising HCDR1 of SEQ ID NO: 107, a HCDR2 of SEQ ID NO: 85, and a HCDR3 of SEQ ID NO: 10;

(e) an antibody variable light chain amino acid sequence comprising LCDR1 of SEQ ID NO: 77, a LCDR2 of SEQ ID NO: 12, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO: 10;

(f) an antibody variable light chain amino acid sequence comprising LCDR1 of SEQ ID NO: 79, a LCDR2 of SEQ ID NO: 12, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO: 10;

(g) an antibody variable light chain amino acid sequence comprising LCDR1 of SEQ ID NO: 79, a LCDR2 of SEQ ID NO: 80, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO:10;

(h) an antibody variable light chain amino acid sequence comprising LCDR1 of SEQ ID NO: 81, a LCDR2 of SEQ ID NO: 80, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO:10; and

(i) an antibody variable light chain amino acid sequence comprising LCDR1 of SEQ ID NO: 82, a LCDR2 of SEQ ID NO: 80, and a LCDR3 of SEQ ID NO: 78; and an antibody variable heavy chain amino acid sequence comprising HCDR1 of SEQ ID NO:107, a HCDR2 of SEQ ID NO: 85, and a HCDR3 of SEQ ID NO: 10.

12. A human antibody or antigen binding fragment thereof that binds to human IL-13 wherein the amino acid sequences comprise a variable light chain region and a variable heavy chain region selected from the group comprising

(a) an antibody variable light chain amino acid sequence comprising SEQ ID NO: 86 and an antibody variable heavy chain amino acid sequence comprising SEQ ID NO: 87;

(b) an antibody variable light chain amino acid sequence comprising SEQ ID NO: 88 and an antibody variable heavy chain amino acid sequence comprising SEQ ID NO: 89;

(c) an antibody variable light chain amino acid sequence comprising SEQ ID NO: 90 and an antibody variable heavy chain amino acid sequence comprising SEQ ID NO: 91;

(d) an antibody variable light chain amino acid sequence comprising SEQ ID NO: 92 and an antibody variable heavy chain amino acid sequence comprising SEQ ID NO: 93;

(e) an antibody variable light chain amino acid sequence comprising SEQ ID NO: 94 and an antibody variable heavy chain amino acid sequence comprising SEQ ID NO: 95;

(f) an antibody variable light chain amino acid sequence comprising SEQ ID NO: 96 and an antibody variable heavy chain amino acid sequence comprising SEQ ID NO: 97;

(g) an antibody variable light chain amino acid sequence comprising SEQ ID NO: 98 and an antibody variable heavy chain amino acid sequence comprising SEQ ID NO: 99;

(h) an antibody variable light chain amino acid sequence comprising SEQ ID NO: 100 and an antibody variable heavy chain amino acid sequence comprising SEQ ID NO: 101;

(i) an antibody variable light chain amino acid sequence comprising SEQ ID NO: 102 and an antibody variable heavy chain amino acid sequence comprising SEQ ID NO: 103, and

(j) an antibody variable light chain amino acid sequence comprising SEQ ID NO: 104 and an antibody variable heavy chain amino acid sequence comprising SEQ ID NO: 105.

13. A human antibody or antigen binding fragment thereof that binds to human IL-13 wherein the amino acid sequences

(a) comprises a light chain selected from the group comprising SEQ ID NO: 22, SEQ ID NO: 24, SEQ ID NO: 26, SEQ ID NO: 28, SEQ ID NO: 30, SEQ ID NO: 33, SEQ ID NO: 35, SEQ ID NO: 37; SEQ ID NO: 39, SEQ ID NO: 41; SEQ ID NO: 43, SEQ ID NO: 45, SEQ ID NO: 47, SEQ ID NO: 49, SEQ ID NO: 51, SEQ ID NO: 54, SEQ ID NO: 56, SEQ ID NO: 58, SEQ ID NO: 60, SEQ ID NO: 62, SEQ ID NO: 65, SEQ ID NO: 67, SEQ ID NO: 69, SEQ ID NO: 71, and SEQ ID NO:73, and

(b) a heavy chain selected from the group comprising SEQ ID NO: 23, SEQ ID NO: 25, SEQ ID NO: 27, SEQ ID NO: 29, SEQ ID NO: 31, SEQ ID NO: 32. SEQ ID NO: 34, SEQ ID NO: 36; SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 53, SEQ ID NO: 55. SEQ ID NO: 57, SEQ ID NO: 59, SEQ ID NO: 61, SEQ ID NO: 63, SEQ ID NO: 64, SEQ ID NO: 66, SEQ ID NO: 68, SEQ ID NO: 70, and SEQ ID NO: 72.

14. An antibody of comprising a light chain and a heavy chain having the amino acid sequences embodiments 1-14, 20, 25-27.
15. A nucleic acid sequence encoding an antibody or antibody fragment thereof embodiments 1-14
16. A vector comprising the nucleic acid sequence encoding an antibody or antibody fragment thereof embodiments 15, 20, 25-27
17. A host cell comprising the vector of claim 16.
18. The host cell of embodiment 17 wherein the host cell is a CHO cell or a SP2/0 cell
19. The host cell of embodiment 18 wherein the host cell is a CHO cell.
20. An antibody or antibody fragment produced by the host cell of embodiments 18-19.
21. A pharmaceutical composition comprising the antibody or antibody binding fragment of embodiment 20.
22. A method of producing an antibody or fragment thereof by culturing the host cells of embodiments 17-19.
23 A method of treating a patient suffering from COPD, emphysema, asthma, or atopic dermatitis by administering and effective amount of the antibodies or fragments thereof of embodiments 20, to the patient.
24 A method of treating a patient suffering from COPD, emphysema, asthma, or atopic dermatitis by administering and effective amount of the pharmaceutical composition of embodiments 21 to the patient.
25 The antibodies of embodiment 20 wherein there is a half-life extension mutation.
26 The antibodies of embodiment 25 wherein the half-life extension mutation is a mutation in the Fc at Eu positions M252Y, S254T, and T256E.
27 The antibodies of embodiment 25 wherein the half-life extension mutation is a mutation in the Fc at complement hexamer disrupting mutation in the Fc at Eu position S583K.

### BRIEF DESCRIPTION OF THE DRAWINGS

The invention will be better understood from the Detailed Description and from the appended drawings, which are meant to illustrate and not to limit the invention.

FIG. 1 shows a plot of the relative antibody concentration against neutralization data for each well. The data was used to identify wells with the highest potency antibodies.

FIG. 2 is a plot depicting the relationship of antigen coating—no this is a plot of ELISA OD of each antibody sample at 31 ng/mL Ag coating versus the concentration of antibody.

FIG. 3 is a graph showing the percent inhibition of IL-13 induced eotaxin release by recombinant antibodies 643 and 731 compared to an isotype matched control.

FIG. 4 is a bar graph comparing the ability of IL-13 or IL-13Q110R to inhibit binding of 731 or 623 to IL-13 coated ELISA plates.

FIG. 5A is a bar graph comparing on cell receptor competition between antibody 643 and an isotype control. Perhaps make this 5B

FIG. 5B is a bar graph comparing on cell receptor competition between antibody 731 and an isotype control. Make this 5D

FIG. 5C is a cartoon depicting the protocol and various predicted results from FIG. 5A. Make this 5A

FIG. 5D is a cartoon depicting the protocol and various predicted results from FIG. 5B. Make this 5C. These changes might make it easier to follow.

FIG. 6A shows the alignment of a phage-display derived peptide recognized by antibody 693 and part of IL-13 sequence.

FIG. 6B is a chart showing the secondary structure of IL-13 and indicates which regions of human IL-13 were replaced with mouse IL-13 for the construction of the chimeric proteins.

FIG. 7 is a chart depicting the various bins in which the various antibodies can be grouped.

FIG. 8A and FIG. 8B are bar graphs showing that CD4⁺ T cells from humanized IL-13 mice produce human IL-13 but not murine IL-13.

FIG. 9 is a graph demonstrating that anti-IL-13 antibodies 731 and 623 inhibit airway hyperresponsiveness.

FIG. 10 is a bar graph demonstrating that 731 and 623 inhibit mucus production.

FIG. 11 shows the crystal structure of the interaction between an affinity matured anti-IL-13 antibody and the IL-13.

FIG. 12A shows a detail of the crystal structure of the interaction between an affinity matured anti-IL-13 antibody and the IL-13.

FIG. 12B shows a detail of the crystal structure of the interaction between an affinity matured anti-IL-13 antibody and the IL-13.

FIG. 13 shows details of the crystal structure of the interaction between an affinity matured anti-IL-13 antibody and the IL-13.

FIG. 14 is a chart showing high affinity anti-IL13 antibody amino acid sequences with half-life extension mutations.

### DETAILED DESCRIPTION

Embodiments of the invention relate isolated antibodies that bind to IL-13 and methods of using those antibodies to treat diseases in humans. Preferably the antibodies are fully human neutralizing monoclonal antibodies that bind to IL-13 with high affinity, high potency, or both. In one embodiment, the antibodies or antibody fragments specifically bind to regions of the IL-13 molecule that prevent it from signaling through the IL-13 receptor complex.

In addition, embodiments of the invention include methods of using these anti-IL-13 antibodies as a diagnostic agent or treatment for a disease. For example, the antibodies are useful for treating asthma, including both allergic (atopic) and non-allergic (non-atopic), bronchial asthma, chronic bronchitis, emphysema, chronic obstructive pulmonary disease (COPD), hay fever, rhinitis, urticaria, angioedema, allergic dermatitis, including contact dermatitis, Stevens-Johnson syndrome, anaphylatctic shock, food allergies, keratitis, conjunctivitis, steroid-resistant nephritic syndrome, mastocytosis, fibrotic disease such as lung fibrosis, including idiopathic pulmonary fibrosis, cystic fibrosis, bleomycin-induced fibrosis, hepatic fibrosis and systemic sclerosis, cancers, such as Hodgkin's disease, B-cell proliferative disorders such as B-cell lymphoma, particularly mediastinal large B-cell lymphoma, B-cell leukemias, ovarian carcinoma, diseases characterized by non-malignant B-cell proliferation such as systemic lupus erythematosus, rheumatoid arthritis, chronic active hepatitis and cryoglobulinemias, high levels of autoantibodies, such as hemolytic anemia, thrombocytopenia, phospholipids syndrome and pemphigus, inflammatory bowel disease and graft-versus-host disease.

In association with such treatment, embodiments of the invention include articles of manufacture comprising the antibodies. An embodiment of the invention is an assay kit comprising IL-13 antibodies that is used to screen for diseases or disorders associated with IL-13 activity.

The nucleic acids described herein, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully below.

In one aspect, methods of identifying these antibodies are provided. In one embodiment, the method involves an eotaxin release assay.

In one aspect, antibodies that bind to a variant of IL-13 are also provided. Especially relevant are those antibodies that bind to an IL-13 variant with a Glutamine at position 110 of the endogenous IL-13 polypeptide.

In an embodiment, a mouse that is humanized for human IL-13 is provided. This mouse is useful for providing a test subject for airway hyperresponsiveness and inhibition of mucus production.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Generally, nomenclatures utilized in connection with, and techniques of, cell and tissue culture, molecular biology, and protein and oligo- or polynucleotide chemistry and hybridization described herein are those well-known and commonly used in the art, as described in various general and more specific references such as those that are cited and discussed throughout the present specification. See e.g. Singleton et al., Dictionary of Microbiology and Molecular Biology 2^{nd} ed., J. Wiley & Sons (New York, N.Y. 1994); Sambrook et al. Molecular Cloning: A Laboratory Manual (2d ed., Cold Spring Harbor Laboratory Press, Cold Spring Harbor, N.Y. (1989)), which is incorporated herein by reference. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. Standard techniques are also used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Pres, N Y, 1989). A used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Antibodies are heterotetrameric glycoproteins of about 150,000 daltons, composed of two identical and substantially full-length light (L) chains and two identical and substantially heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985); Novotny and Haber, Proc. Nal. Acad. Sci. U.S.A. 82:4592 (1985); Chothia et al., Nature 342:877-883 (1989)).

“Antibody fragments” include fragments of an antibody that bind the target antigen. Examples include Examples of antibody fragments include Fab, Fab′, F(ab′)₂, and Fv fragments.

“Antigen binding proteins” as used herein means a protein that specifically binds a specified antigen that are derived from antibodies. Examples of antigen binding proteins include but are not limited to antibodies, antibody fragments, antibody constructs, fusion proteins, bispecific antibodies, and scFv proteins.

An antigen binding protein is said to “specifically bind” to its antigen when the antigen binding protein binds its antigen with a dissociation constant (KD) is ≤10⁻⁷ M as measured via a surface plasma resonance technique (e.g., BIACore, GE-Healthcare Uppsala, Sweden) or Kinetic Exclusion Assay (KinExA, Sapidyne, Boise, Id.).

Antigen binding proteins of the invention can be neutralizing and inhibit binding of IL-13 to a signaling receptor, such as IL-13 receptor alpha-1 (IL-13Rα1) by at least 60% or 80%, and more usually greater than about 85% (as measured in an in vitro competitive binding assay). In an embodiment, the antibodies also inhibit binding to the decoy receptor IL-13Rα2, while in other embodiments the ability of IL-13 to bind IL-13Rα2 is maintained upon antibody binding to IL-13.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and terminal or internal amino acid sequence by use of a spinning cup sequenator, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “neutralizing antibody” is an antibody molecule which is able to eliminate or significantly reduce an effector function of a target antigen to which it binds. Accordingly, a “neutralizing” IL-13 antibody is capable of eliminating or significantly reducing an effector function, such as IL-13 signaling activity through the IL-13 receptor. In one embodiment, a neutralizing antibody will reduce an effector function by 1-10, 10-20, 20-30, 30-50, 50-70, 70-80, 80-90, 90-95, 95-99, 99-100%.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcRs expression on hematopoietic cells is summarized in Table 3 on page 464 of Ravetch and Kinet, Annu. Rev. Immunol. 9:457-492 (1991). To assess ADCC activity of a molecule of interest, an in vitro ADCC assay, such as that described in U.S. Pat. No. 5,500,362, or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1988).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the Ig light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al. (1991)). The constant domains are not involved directly in binding an antibody to an antigen, but exhibit various effector functions, such as participation of the antibody in antibody-dependent cellular toxicity.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites.

“Fab” when used herein refers to a fragment of an antibody which comprises the constant domain of the light chain and the CH1 domain of the heavy chain.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fusion protein” refers to protein that comprises an antibody fragment bound to another protein.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (Li), 50-62 (L2), and 89-97 (L3) in the light chain variable domain and 31-55 (H1), 50-65 (H2) and 95-102(H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (e.g. residues 26-32(L1), 50-52(L2) and 91-96 (L3) in the light chain variable domain and 26-32((H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

The term “epitope” is used to refer to binding sites for antibodies on protein antigens. Epitopic determinants usually consist of chemically active surface groupings of molecules such as amino acids or sugar side chains and usually have specific three-dimensional structural characteristics, as well as specific charge characteristics. An antibody is said to bind an antigen when the dissociation constant is ≤1 μM, preferably ≤100 nM and most preferably ≤10 nM. An increased or greater equilibrium constant (“K_{D}”) means that there is less affinity between the epitope and the antibody. In other words, that the antibody and the epitope are less favorable to bind or stay bound together. A decrease of lower equilibrium constant means that there is a higher affinity between the epitope and the antibody. In other words, it is more likely that the antibody and the epitope will bind or stay bound together. An antibody with a K_{D} of “no more than” a certain amount means that the antibody will bind to the epitope with the given affinity, or more strongly (or tightly).

While K_{D} describes the binding characteristics of an epitope and an antibody, “potency” describes the effectiveness of the antibody itself for a function of the antibody. A relatively low K_{D} does not automatically mean a high potency. Thus, antibodies can have a relatively low K_{D} and a high potency (e.g., they bind well and alter the function strongly), a relatively high K_{D} and a high potency (e.g., they don't bind well but have a strong impact on function), a relatively low K_{D} and a low potency (e.g., they bind well, but not in a manner effective to alter a particular function) or a relatively high K_{D} and a low potency (e.g., they simply do not bind to the target well). In one embodiment, high potency means that there is a high level of inhibition with a low concentration of antibody. In one embodiment, an antibody is potent or has a high potency when its IC₅₀ is a small value, for example, 130-110, 110-90, 90-60, 60-30, 30-25, 25-20, 20-15, or less pM.

“Substantially,” unless otherwise specified in conjunction with another term, means that the value can vary within the any amount that is contributable to errors in measurement that may occur during the creation or practice of the embodiments. “Significant” means that the value can vary as long as it is sufficient to allow the claimed invention to function for its intended use.

The term “amino acid” or “amino acid residue,” as used herein, refers to naturally occurring L amino acids or to D amino acids as described further below with respect to variants. The commonly used one and three-letter abbreviations for amino acids are used herein (Bruce Alberts et al., Molecular Biology of the Cell, Garland Publishing, Inc., New York (3d ed. 1994)).

The term “mAb” refers to monoclonal antibody.

The term “human antibody” refers to an antibody that where most of the antibody sequence (at least 95%) is derived from the human genome.

The term “XENOMOUSE® refers to strains of mice which have been engineered to contain 245 kb and 190 kb-sized germline configuration fragments of the human heavy chain locus and kappa light chain locus, as described in Green et al. Nature Genetics 7:13-21(1994), incorporated herein by reference. The XENOMOUSE® strains are available from Abgenix, Inc. (Fremont, Calif.).

The term “XENOMAX®” refers use of to the use of the “Selected Lymphocyte Antibody Method” (Babcook et al., Proc. Natl. Acad. Sci. USA, 93:7843-7848 (1996)), when used with XENOMOUSE® animals.

The term “SLAM®” refers to the “Selected Lymphocyte Antibody Method” (Babcook et al., Proc. Natl. Acad Sci. USA. 93:7843-7848 (1996), and Schrader, U.S. Pat. No. 5,627,052), both of which are incorporated by reference in their entireties.

The terms “disease,” “disease state” and “disorder” refer to a physiological state of a cell or of a whole mammal in which an interruption, cessation, or disorder of cellular or body functions, systems, or organs has occurred.

The term “symptom” means any physical or observable manifestation of a disorder, whether it is generally characteristic of that disorder or not. The term “symptoms” can mean all such manifestations or any subset thereof.

The term “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The term “inhibit,” when used in conjunction with a disease or symptom can mean that the antibody can reduce or eliminate the disease or symptom.

The term “patient” includes human and veterinary subjects.

“Administer,” for purposes of treatment, means to deliver to a patient. For example and without limitation, such delivery can be intravenous, intraperitoneal, by inhalation, intramuscular, subcutaneous, oral, topical, transdermal, or surgical.

“Therapeutically effective amount,” for purposes of treatment, means an amount such that an observable change in the patient's condition and/or symptoms could result from its administration, either alone or in combination with other treatment.

A “pharmaceutically acceptable vehicle,” for the purposes of treatment, is a physical embodiment that can be administered to a patient. Pharmaceutically acceptable vehicles can be, but are not limited to, pills, capsules, caplets, tablets, orally administered fluids, injectable fluids, sprays, aerosols, lozenges, neutraceuticals, creams, lotions, oils, solutions, pastes, powders, vapors, or liquids. One example of a pharmaceutically acceptable vehicle is a buffered isotonic solution, such as phosphate buffered saline (PBS).

“Neutralize,” for purposes of treatment, means to partially or completely suppress chemical and/or biological activity.

“Down-regulate,” for purposes of treatment, means to lower the level of a particular target composition.

“Mammal” for purposes of treatment refers to any animal classified as a mammal, including humans, domestic and farm animals, and zoo, sports, or pet animals, such as monkeys, dogs, horses, cats, cows, etc.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides can be either sense or antisense oligonucleotides.

The term “naturally occurring nucleotide” as used herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

The term “control sequence” as used herein refers to polynucleotide sequences which are necessary to effect the expression and processing of coding sequences to which they are connected. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Polypeptides in accordance with the invention comprise the human heavy chain immunoglobulin molecules represented Tables and 21, by SEQ ID NOs: 2, 6, 10, 14, 18, 22, 26, 30, 34, 38, 42, 46, 50, 54, 58, and 83-105, for example, and the human kappa light chain immunoglobulin molecules represented by SEQ ID NOs 4, 8, 12, 16, 20, 24, 28, 32, 36, 40, 44, 48, 52, 56, 60, and 106-126, for example, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

Unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as alpha-, alpha-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the righthand direction is the carboxy-terminal direction, in accordance with standard usage and convention.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are among those used to describe the sequence relationships between two or more polynucleotide or amino acid sequences: “reference sequence”, “comparison window”, “sequence identity”, “percentage of sequence identity”, “substantial identity”, and “homology.” A “reference sequence” is a defined sequence used as a basis for a sequence comparison. A reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity.

A “comparison window”, as used herein, refers to a conceptual segment of at least about 18 contiguous nucleotide positions or about 6 amino acids wherein the polynucleotide sequence or amino acid sequence is compared to a reference sequence of at least 18 contiguous nucleotides or 6 amino acid sequences and wherein the portion of the polynucleotide sequence in the comparison window may include additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482(1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), GENEWORKS™, or MACVECTOR® software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

Two amino acid sequences or polynucleotide sequences are “homologous” if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least about 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family.

For example, it is reasonable to expect that an isolated replacement of a leucine with an isoleucine or valine, an aspartate with a glutamate, a threonine with a serine, or a similar replacement of an amino acid with a structurally related amino acid will not have amajor effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein.

Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). The foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physiocochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thomton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally occurring sequence deduced, for example, from a full-length cDNA sequence. Fragments typically are at least 5, 6, 8 or 10 amino acids long, preferably at least 14 amino acids long, more preferably at least 20 amino acids long. In other embodiments polypeptide fragments are at least 25 amino acids long, more preferably at least 50 amino acids long, and even more preferably at least 70 amino acids long.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

### Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. (See generally, Fundamental Immunology Ch. 7 (Paul, W., ed., 2nd ed. Raven Press, N.Y. (1989)), incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. (See, e.g., Songsivilai & Lachmann Clin. Exp. Immunol. 79: 315-321 (1990), Kostelny et al. J. Immunol. 148:1547-1553 (1992)). Production of bispecific antibodies can be a relatively labor-intensive process compared with production of conventional antibodies and yields and degree of purity are generally lower for bispecific antibodies. Bispecific antibodies do not exist in the form of fragments having a single binding site (e.g., Fab, Fab′, and Fv).

### Human Antibodies and Humanization of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of human antibody function into a rodent so that the rodent produces fully human antibodies.

One method for generating fully human antibodies is through the use of XENOMOUSE® strains of mice which have been engineered to contain 245 kb and 190 kb-sized germline configuration fragments of the human heavy chain locus and kappa light chain locus. See Green et al. Nature Genetics 7:13-21(1994). The XENOMOUSE® strains are available from Abgenix, Inc. (Fremont, Calif.).

The production of the XENOMOUSE® is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm Intemational, Inc., have utilized a “minilocus” approach. In the minilocus approach, an exogenous Ig locus is mimicked through the inclusion of pieces (individual genes) from the Ig locus. Thus, one or more V_{H} genes, one or more DH genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference in their entireties.

Human anti-mouse antibody (HAMA) responses have also led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against multimeric enzymes in order to vitiate concerns and/or effects of HAMA or HACA response.

### Preparation of Antibodies

Antibodies, as described herein, were prepared using the XENOMOUSE® technology, as described below. Such mice are capable of producing human immunoglobulin molecules and antibodies and are deficient in the production of murine immunoglobulin molecules and antibodies. Technologies utilized for achieving the same are disclosed in the patents, applications, and references referred to herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through use of such technology, fully human monoclonal antibodies to IL-13 were produced, as described in detail below. Essentially, XENOMOUSE® lines of mice were immunized with human IL-13, lymphatic cells (such as B-cells) were recovered from mice that expressed antibodies, and the recovered cell lines were fused with a myeloid-type cell line to prepare immortal hybridoma cell lines. These hybridoma cell lines were screened and selected to identify hybridoma cell lines that produced antibodies specific to the IL-13. Further, provided herein are characterization of the antibodies produced by such cell lines, including nucleotide and amino acid sequence analyses of the heavy and light chains of such antibodies.

Alternatively, instead of being fused to myeloma cells to generate hybridomas, the recovered cells, isolated from immunized XENOMOUSE® lines of mice, can be screened further for reactivity against the initial antigen, preferably human IL-13. Such screening includes an ELISA with the desired IL-13 protein and functional assays such as IL-13-induced eotaxin-1 production. Single B cells secreting antibodies that specifically bind to IL-13 can then be isolated using a desired IL-13-specific hemolytic plaque assay (Babcook et al., Proc. Natl. Acad. Sci. USA, i93:7843-7848 (1996)). Cells targeted for lysis are preferably sheep red blood cells (SRBCs) coated with IL-13. In the presence of a B cell culture secreting the immunoglobulin of interest and complement, the formation of a plaque indicates specific IL-13-mediated lysis of the target cells.

The single antigen-specific plasma cell in the center of the plaque can be isolated and the genetic information that encodes the specificity of the antibody isolated from the single plasma cell. Using reverse-transcriptase PCR, the DNA encoding the variable region of the antibody secreted can be cloned. Such cloned DNA can then be further inserted into a suitable expression vector, preferably a vector cassette such as a pcDNA (Invitrogen, Carlsbad, Calif.), more preferably such a pcDNA vector containing the constant domains of immunoglobulin heavy and light chain. The generated vector can then be transfected into host cells, preferably CHO cells, and cultured in conventional nutrient media modified as appropriate for inducing promoters, selecting transformants, or amplifying the genes encoding the desired sequences.

Herein, is described the isolation of multiple single plasma cells that produce antibodies specific to IL-13. Further, the genetic material that encoded an antibody that specifically bound IL-13 was isolated, and that material was introduced into a suitable expression vector and thereafter transfected into host cells.

In general, antibodies produced by the above-mentioned cell lines possessed fully human IgG1 or IgG2 heavy chains with human kappa light chains. The antibodies possessed high affinities, typically possessing KD's of from about 10⁻⁹ through about 10⁻¹³ M, when measured by either solid phase and solution phase.

As mentioned above, anti-IL-13 antibodies can be expressed in cell lines other than hybridoma cell lines. Sequences encoding particular antibodies can be used for transformation of a suitable mammalian host cell, such as a CHO cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed.

Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, Sp2/0 cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with IL-13 binding properties.

### Antibody Sequences

The heavy chain and light chain variable region nucleotide and amino acid sequences of representative human anti-IL-13 antibodies are provided in the sequence listing, the contents of which are summarized in Table 1 below.

TABLE 1

| Description | Sequence | Seq ID NO: |
|---|---|---|
| MMAB7 HCD1 | SYAMS | 8 |
| MMAB7 HCD2 | AFSGWDVSTYYADSVKG | 9 |
| MMAB7 HCD3 | DGLGPYFYNYGMDV | 10 |
| MMAB7 HLD1 | SGDKLGDKYS | 11 |
| MMAB7 HLD2 | HDSKRPS | 12 |
| MMAB7 HLD3 | QAWDSSTYV | 13 |

### Antibody Therapeutics

Anti-IL-13 antibodies have therapeutic value for treating symptoms and conditions related to IL-13 activity. IL-13 has been implicated in a wide variety of diseases and disorders, including inflammatory diseases, cancer, fibrotic disease and diseases characterized by non-malignant cell proliferation. In specific embodiments, the anti-IL-13 antibodies disclosed herein are used in the treatment of inflammatory diseases or disorders such as asthma, including both allergic (atopic) and non-allergic (non-atopic), bronchial asthma, chronic bronchitis, emphysema, chronic obstructive pulmonary disease (COPD), hay fever, rhinitis, urticaria, angioedema, allergic dermatitis, including contact dermatitis, Stevens-Johnson syndrome, anaphylactic shock, food allergies, keratitis, conjunctivitis, steroid-resistant nephritic syndrome. In other embodiments they are used to treat mastocytosis. In still other embodiments they are used to treat fibrotic disease such as lung fibrosis, including idiopathic pulmonary fibrosis, cystic fibrosis, bleomycin-induced fibrosis, hepatic fibrosis and systemic sclerosis. In further embodiments the anti-IL-13 antibodies are used to treat cancers, such as Hodgkin's disease, B-cell proliferative disorders such as B-cell lymphoma, particularly mediastinal large B-cell lymphoma, B-cell leukemias, ovarian carcinoma.

In still further embodiments the anti-IL-13 antibodies are used to treat diseases characterized by non-malignant B-cell proliferation such as systemic lupus erythematosus, rheumatoid arthritis, chronic active hepatitis and cryoglobulinemias; disease characterized by high levels of autoantibodies, such as hemolytic anemia, thrombocytopenia, phospholipids syndrome and pemphigus; inflammatory bowel disease; and graft-versus-host disease.

If desired, the isotype of an anti-IL-13 antibody can be switched, for example to take advantage of a biological property of a different isotype. For example, in some circumstances it may be desirable for the therapeutic antibodies against IL-13 to be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

By way of example, the anti-IL-13 antibodies discussed herein are human antibodies. If an antibody possessed desired binding to IL-13, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

Biologically active antibodies that bind IL-13 are preferably used in a sterile pharmaceutical preparation or formulation to reduce the activity of IL-13. Anti-IL-13 antibodies preferably possess adequate affinity to potently suppress IL-13 activity to within the target therapeutic range. The suppression preferably results from the ability of the antibody to interfere with the binding of IL-13 to a signaling receptor, such as IL-13Ra1 (also known as, IL-13 Rα1, Rα1, IL-13R alpha 1, IL-13 receptor alpha 1, or other similar terms). In other embodiments the antibody may suppress IL-13 activity by interfering with the ability of IL-13 to signal through the receptor, even if it is able to bind. For example, the antibody may prevent interaction of the IL-13Ra1 with a co-receptor that is necessary for signaling, such as the IL-4 receptor alpha chain. In some embodiments the antibody is able to prevent IL-13 activity through a signaling receptor while allowing for IL-13 binding to a decoy receptor, such as IL-13Ra2. In this case, binding to the decoy receptor may allow clearance of the bound IL-13 and enhance the ability of the antibody to suppress IL-13 activity.

When used for in vivo administration, the antibody formulation is preferably sterile. This is readily accomplished by any method know in the art, for example by filtration through sterile filtration membranes. The antibody ordinarily will be stored in lyophilized form or in solution. Sterile filtration may be performed prior to or following lyophilization and reconstitution.

Therapeutic antibody compositions generally are placed into a container having a sterile access port, for example, an intravenous solution bag or vial having an adapter that allows retrieval of the formulation, such as a stopper pierceable by a hypodermic injection needle.

The modality of antibody administration is in accord with known methods, e.g., injection or infusion by subcutaneous, intravenous, intraperitoneal, intracerebral, intradermic, intramuscular, intraocular, intraarterial, intrathecal, or intralesional routes, or by inhalation or by sustained release systems as noted below. In some situations the antibody is preferably administered by infusion or by bolus injection. In other situations a therapeutic composition comprising the antibody can be administered through the nose or lung, preferably as a liquid or powder aerosol (lyophilized). The composition may also be administered intravenously, parenterally or subcutaneously as desired. When administered systemically, the therapeutic composition should be sterile, pyrogen-free and in a parenterally acceptable solution having due regard for pH, isotonicity, and stability. These conditions are known to those skilled in the art.

Antibodies for therapeutic use, as described herein, are typically prepared with suitable carriers, excipients, and other agents that are incorporated into formulations to provide improved transfer, delivery, tolerance, and the like. Briefly, dosage formulations of the antibodies described herein are prepared for storage or administration by mixing the antibody having the desired degree of purity with one or more physiologically acceptable carriers, excipients, or stabilizers. These formulations may include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, carbowax (polyethylene glycols of various molecular weights), semi-solid gels, and semi-solid mixtures containing carbowax. The formulation may include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Other acceptable carriers, excipients and stabilizers are well known to those of skill in the art. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J. Pharm. Sci. 89(8):967-78 (2000), Powell et al. “Compendium of excipients for parenteral formulations” PDA J. Pharm. Sci. Technol. 52:238-311 (1998) and the citations therein for additional information.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

The antibodies can also be administered in and released over time from sustained-release preparations. Suitable examples of sustained-release preparations include semipermeable matrices of solid hydrophobic polymers containing the polypeptide. The matrices may be in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Nal. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient may be determined by the attending physician. In determining the appropriate dosage the physician may take into consideration various factors known to modify the action of therapeutics, including, for example, severity and type of disease, body weight, sex, diet, time and route of administration, other medications and other relevant clinical factors. Therapeutically effective dosages may be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.001 mg/kg to up to 100 mg/kg or more, depending on the factors mentioned above. Typically, the clinician will administer the therapeutic antibody until a dosage is reached that achieves the desired effect. The progress of this therapy is easily monitored by conventional assays.

It is expected that the antibodies described herein will have therapeutic effect in treatment of symptoms and conditions resulting from or related to the activity of IL-13.

### Design and Generation of Other Therapeutics

In accordance with the present invention and based on the activity of the antibodies that are produced and characterized herein with respect to IL-13, advanced antibody therapeutics may be employed to treat specific diseases. These advanced therapeutics may include bispecific antibodies, immunotoxins, radiolabeled therapeutics, peptide therapeutics, gene therapies, particularly intrabodies, antisense therapeutics, and small molecules.

In connection with the generation of advanced antibody therapeutics, where complement fixation is a desirable attribute, it may be possible to sidestep the dependence on complement for cell killing through the use of bispecifics, immunotoxins, or radiolabels, for example.

For example, bispecific antibodies can be generated that comprise (i) two antibodies, one with a specificity to IL-13 and another to a second molecule, that are conjugated together, (ii) a single antibody that has one chain specific to IL-13 and a second chain specific to a second molecule, or (iii) a single chain antibody that has specificity to both IL-13 and the other molecule. Such bispecific antibodies can be generated using techniques that are well known; for example, in connection with (i) and (ii) see e.g., Fanger et al. Immunol Methods 4:72-81 (1994) and Wright and Harris, supra, and in connection with (iii) see e.g., Traunecker et al. Int. J. Cancer (Suppl) 7:51-52(1992). In each case, the second specificity can be made as desired. For example, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)).

In some embodiments, an article of manufacture is provided comprising a container, comprising a composition containing an anti-IL-13 antibody, and a package insert or label indicating that the composition can be used to treat disease mediated by IL-13. Preferably a mammal and, more preferably, a human, receives the anti-IL-13 antibody. In preferred embodiments, the disease to be treated is selected from the group consisting of asthma, including both allergic (atopic) and non-allergic (non-atopic), bronchial asthma, chronic bronchitis, emphysema, chronic obstructive pulmonary disease (COPD), hay fever, rhinitis, urticaria, angioedema, allergic dermatitis, including contact dermatitis, Stevens-Johnson syndrome, anaphylatctic shock, food allergies, keratitis, conjunctivitis, steroid-resistant nephritic syndrome, mastocytosis, fibrotic disease such as lung fibrosis, including idiopathic pulmonary fibrosis, cystic fibrosis, bleomycin-induced fibrosis, hepatic fibrosis and systemic sclerosis, cancers, such as Hodgkin's disease, B-cell proliferative disorders such as B-cell lymphoma, particularly mediastinal large B-cell lymphoma, B-cell leukemias, ovarian carcinoma, diseases characterized by non-malignant B-cell proliferation such as systemic lupus erythematosus, rheumatoid arthritis, chronic active hepatitis and crioglobulnimias, high levels of autoantibodies, such as hemolytic anemia, thrombocytopenia, phospholipids syndrome and pemphigus, inflammatory bowel disease and graft-versus-host disease.

In some embodiments an anti-IL-13 antibody is used to treat asthma. In a particular embodiment the antibody is the 623 antibody or variants thereof described herein. In another particular embodiment the antibody is the 731 antibody or variants thereof described herein.

### EXAMPLES

The following examples, including the experiments conducted and results achieved are provided for illustrative purposes only and are not to be construed as limiting upon the teachings herein.

### Example 1: Antibody Generation

### IL-13 and IL-13 Antigen Preparation

The following IL-13 peptides were used in the experiments described below.

Recombinant Human IL-13 (R&D 213-IL-005;
SEQ ID NO: 1):
GPVPPSTALRELIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGQFN
Recombinant Human IL-13 (Peprotech 200-13;
SEQ ID NO: 2):
SPGPVPPSTALRELIEELVNITQNQKAPLCNGSMVWSINLTAGMYCAALE
SLINVSGCSAIEKTQRMLSGFCPHKVSAGQFSSLHVRDTKIEVAQFVKDL
LLHLKKLFREGRFN
Recombinant Human IL-13 (Peprotech 200-13A;
SEQ ID NO: 3):
MSPGPVPPSTALRELIEELVNITQNQKAPLCNGSMVWSINLTAGMYCAAL
ESLINVSGCSAIEKTQRMLSGFCPHKVSAGQFSSLHVRDTKIEVAQFVKD
LLLHLKKLFREGQFN
Human IL-13-human Fc fusion protein (with leader
sequence; SEQ ID NO: 4):
MALLLTTVIALTCLGGFASPGPVPPSTALRELIEELVNITQNQKAPLCNG
SMVWSINLTAGMYCAALESLINVSGCSAIEKTQRMLSGFCPHKVSAGQFS
SLHVRDTKIEVAQFVKDLLLHLKKLFREGRFNEPKSCDKTHTCPPCPAPE
LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALH
NHYTQKSLSLSPGK
Human IL-13-rabbit Fc fusion protein (with leader
sequence; SEQ ID NO: 5):
MALLLTTVIALTCLGGFASPGPVPPSTALRELIEELVNITQNQKAPLCNG
SMVWSINLTAGMYCAALESLINVSGCSAIEKTQRMLSGFCPHKVSAGQFS
SLHVRDTKIEVAQFVKDLLLHLKKLFREGRFNRYLDKTVAPSTCSKPTCP
PPELLGGPSVFIFPPKPKDTLMISRTPEVTCVVVDVSQDDPEVQFTWYIN
NEQVRTARPPLREQQFNSTIRVVSTLPIAHQDWLRGKEFKCKVHNKALPA
PIEKTISKARGQPLEPKVYTMGPPREELSSRSVSLTCMINGFYPSDISVE
WEKNGKAEDNYKTTPAVLDSDGSYFLYNKLSVPTSEWQRGDVFTCSVMHE
ALHNHYTQKSISRSPGK
Human IL-13-Mouse IL-13 Helix A (underlined;
SEQ ID NO: 6):
MHPLLNPLLLALGLMALLLTTVIALTCLGGFASPGPVPRSVSLPLTLKEL
IEELVNITQNQKAPLCNGSMVWSINLTAGMYCAALESLINVSGCSAIEKT
QRMLSGFCPHKVSAGQFSSLHVRDTKIEVAQFVKDLLLHLKKLFREGQFN
Human IL-13-Mouse IL-13 Helix B (underlined;
SEQ ID NO: 7):
MHPLLNPLLLALGLMALLLTTVIALTCLGGFASPGPVPPSTALRELIEEL
VNITQNQKAPLCNGSMVWSINLTAGGFCVALDSLTNVSGCSAIEKTQRML
SGFCPHKVSAGQFSSLHVRDTKIEVAQFVKDLLLHLKKLFREGQFN
Human IL-13-Mouse IL-13 Helix C (underlined;
SEQ ID NO: 68):
MHPLLNPLLLALGLMALLLTTVIALTCLGGFASPGPVPPSTALRELIEEL
VNITQNQKAPLCNGSMVWSINLTAGMYCAALESLINVSGCSAIYRTQRIL
HGLCPHKVSAGQFSSLHVRDTKIEVAQFVKDLLLHLKKLFREGQFN
Human IL-13-Mouse IL-13 Helix D (underlined;
SEQ ID NO: 69):
MHPLLNPLLLALGLMALLLTTVIALTCLGGFASPGPVPPSTALRELIEEL
VNITQNQKAPLCNGSMVWSINLTAGMYCAALESLINVSGCSAIEKTQRML
SGFCPHKVSAGQFSSLHVRDTKIEVAHFITKLLSYTKQLFRHGQQFN

As will be appreciated by one of skill in the art, only a subset of the above residues may actually be involved in the formation ofan epitope. For example, in SEQ ID NOs: 66-69 above, the epitopes may actually be the helix portion of each peptide (the underlined section).

### Immunization of Animals

Monoclonal antibodies against IL-13 were developed by immunizing XenoMouse® mice (XenoMouse® XMG2L3 and XenoMouse® XMG2, Abgenix, Inc. Fremont, Calif.). The human IL-13-human Fc fusion protein (SEQ ID NO: 64) or human IL-13-rabbit Fc fusion protein (SEQ ID NO: 65) was used as the immunogen for antibody generation. Each mouse was immunized via the footpad route of administration. The animals were immunized on days 0, 4, 7, 11, 14, 18, 21 and 25. The initial immunization was with 10 ug of antigen in CpG/Alum per mouse. Subsequent boosts were with 5 ug of antigen in CpG/Alum per mouse. The final boost on day 25 was with 5 ug of antigen in PBS without adjuvant per mouse. The animals were bled on day 20 to obtain sera for determination of titer as described below.

### Titer Analysis

Titer was determined using a standard protocol. Briefly, Costar 3368 plates were coated with either IL-13 rabbit Fc fusion protein (SEQ ID NO: 65) or full-length rabbit antibody overnight at 4° C. The plates were washed using Titertek Program ADG9, dried, and blocked with 250 μl 1% no fat skim milk/1×PBS. Following blocking, the plates were washed again using Titertek Program ADGP and dried. The sera to be tested was titrated vertically 1:2 in duplicate from a 1:100 initial dilution. The samples were run in 1% non fat skim milk/1×PBS at 50 ul/well and incubated for 1 h at room temperature.

After washing using Titertek Program ADG9 and drying, the plates were incubated for 1 hour at room temperature with a secondary rabbit anti-human Fc antibody conjugated to POD (1:8000 dilution; 50 μL/well) with minimal cross-reactivity to rabbit Fc in 1% no fat skim milk/1×PBS. Plates were then washed a final time using Titertek Program ADG9 and dried. POD substrate one-step TMB solution (50 μl/well) was added and allowed to develop for 30 minutes at room temperature. The reaction was stopped with 1 N HCL (50 μ/well) and the optical density was read immediately with a Titertek Plate reader.

Three animals with high titers for the IL-13 immunogen, as shown in Table 2, were selected for harvest.

TABLE 2

Coating

| Mouse | IL-13 Rb Fc | RbIgG |
|---|---|---|
| 1 | 3855 | <100 |
| 2 | 5444 | <100 |
| 3 | >6400 | 268 |
| naïve | <100 | <100 |

### Primary Screen

The hyperimmune animals were harvested and CD19+ B-cells were isolated for subsequent B cell culture. The cells were induced to proliferate and terminally differentiate into plasma cells. Supernatants from these plasma cells were screened by ELISA to identify primary wells containing anti-IL-13-specific antibodies. The cultures were commonly run with 50 to 500 CD19+ B cells per well to allow the identification of monoclonal antigen-specific B cell cultures.

Briefly, IL-13-RbFc was coated onto Costar 3368% well plates at 1 ug/mL overnight. Each plate was washed 5 times with dH₂O and 40 μL of 1% milk in PBS were added to the plate. Subsequently, 10 μL of B cell supernatant was added to each well. After an hour at room temperature, the plates were again washed 5 times with dH₂O. To each well was added 50 μL of Rabbit anti-Human Fc-HRP with minimum anti-rabbit cross-reactivity (Jackson Laboratories; 1:8000 dilution). After 1 hour at room temperature, the plates were again washed 5 times with dH₂O and 50 μL of TMB substrate (Neogen) were added to each well. The reaction was stopped after 30 minutes by the addition of 50 μL of 1 N hydrochloric acid to each well and the plates were read at wavelength 450 nm.

Representative data resulting from the primary screen is shown below in Table 3. Positive wells were identified as those that were found to have a signal at least three times that of a control well. A total of 968 positive antigen-specific B cell wells were identified in the primary screen. All of these wells were taken forward for screening in a functional assay, as described below.

TABLE 3

| Plate | Well | Primary O.D. |
|---|---|---|
| 2357 | G11 | 2.598 |
| 2361 | G5 | 3.218 |
| 2372 | B8 | 2.308 |
| 2383 | H5 | 3.05 |
| 2398 | C5 | 2.203 |
| 2401 | G12 | 3.566 |
| 2413 | G11 | 3.347 |
| 2384 | G12 | 4.057 |
| 2388 | A10 | 4.219 |
| 2407 | G11 | 3.448 |

### IL-13-Induced Eotaxin-1 Production Assay

All of the 968 ELISA positive wells were screened twice in an IL-13-induced Eotaxin-1 release assay. The assay was performed such that only wells containing a high concentration of antibody or wells containing high affinity antibody were identified as neutralizing. A total of 78 neutralizing antibodies were identified as neutralizing in this assay. The specific data from several wells of interest are also shown for illustrative purposes in Table 4.

For the assay, half of the area of 96-well assay plates was seeded with 4000 HDFa cells/well in 50 μL of Medium 106 supplemented with low serum growth supplement (Cascade). The plates were then incubated overnight at 37° C. in 5% CO₂. In a separate plate, 12.5 μL sample antibody, negative control or positive control was aliquoted into sterile 96-well assay plates. Approximately 600 μM of IL-13 was prepared in Medium 106 (4× final concentration) and approximately 100 ng/mL TNF-alpha was prepared in Medium 106 (2× final concentration).

To begin the assay, 12.5 μL of IL-13 or media alone was added to each well and allowed to incubate at 37° C. in 5% CO₂ for 1 hr. Following the 1 hr incubation, the media of the HDFa cells was carefully removed using a multichannel pipette. 25 μL of TNF-alpha was added to each well. 25 μL sample/IL-13 was transferred to HDFa/TNF-alpha wells and cells were incubated at 37° C. in 5% CO₂ for 48 hrs.

Following 48 hours of incubation, supernatant from HFDa assay wells was collected into 96-well VEE bottom plate. Samples were centrifuged at 1500 rpm for 5 min.

30 μL of sample was assayed for Eotaxin-1 release in an assay kit (R&D systems) according to standard protocol with the following modifications. (1) 50 μL Capture Ab was coated at 2 μg/mL; (2) 50 μL sample or standard was used (30 μL sample+20 μL media for a final volume of 50 μL); (3) 50 μL of detection Ab was used at 0.1 μg/mL; (4) 50 μL Streptavidin-HRP was used at 0.5 μg/mL; and (5) 50 μL Substrate Solution was used.

TABLE 4

| | | | Eotaxin | | | Eotaxin | |
|---|---|---|---|---|---|---|---|
| | | ELISA | Concentration | % | ELISA | Concentration | % |
| Plate | Well | O.D. | (pg/mL) | Inhibition | O.D. | (pg/mL) | Inhibition |
| 2357 | G11 | 0.429 | 25 | 79 | 0.283 | 13 | 80 |
| 2361 | G05 | 0.393 | 19 | 85 | 0.295 | 15 | 76 |
| 2372 | B08 | 0.532 | 41 | 72 | 0.282 | 13 | 80 |
| 2383 | H05 | 0.42 | 23 | 84 | 0.247 | 6 | 90 |
| 2398 | C05 | 0.34 | 11 | 90 | 0.228 | 3 | 96 |
| 2401 | G12 | 0.564 | 46 | 70 | 0.384 | 31 | 57 |
| 2413 | G11 | 0.401 | 20 | 84 | 0.283 | 13 | 82 |
| 2384 | G12 | 0.517 | 38 | 73 | 0.297 | 15 | 76 |
| 2388 | A10 | 0.459 | 29 | 78 | 0.274 | 11 | 82 |
| 2407 | G11 | 0.469 | 31 | 78 | 0.278 | 12 | 84 |

### High Antigen (HA) Analysis of Anti-IL-13 Specific B Cell Culture Wells

Using an ELISA method, supernatants for concentration of antigen specific antibody were normalized. Using an anti-target (IL-13) antibody of known concentration titrated in parallel, a standard curve was generated and the amount of antigen specific antibody in the supernatant was compared to the standard and its concentration determined, see Table 5 below.

TABLE 5

| | | | | | | Ab Concentration |
|---|---|---|---|---|---|---|
| | | | | | | (ng/ml) Based on |
| | | ELISA OD determined at different | | | | an anti-IL-13 |
| Plate | Well | antibody dilutions | | | | Standard Curve |
| 2357 | G11 | 3.944 | 1.769 | 0.708 | 0.424 | 386 |
| 2361 | G5 | 4.483 | 2.345 | 0.794 | 0.438 | 532 |
| 2372 | B8 | 3.209 | 1.238 | 0.552 | 0.373 | 240 |
| 2383 | H5 | 4.389 | 2.361 | 0.768 | 0.438 | 523 |
| 2398 | C5 | 2.057 | 0.752 | 0.383 | 0.324 | 114 |
| 2401 | G12 | 4.312 | 2.285 | 0.796 | 0.441 | 521 |
| 2413 | G11 | 3.977 | 1.783 | 0.648 | 0.415 | 360 |
| 2384 | G12 | 4.639 | 3.132 | 1.072 | 0.528 | 856 |
| 2388 | A10 | 4.689 | 3.23 | 1.261 | 0.612 | 1049 |
| 2407 | G11 | 4.891 | 2.9 | 1.072 | 0.537 | 824 |

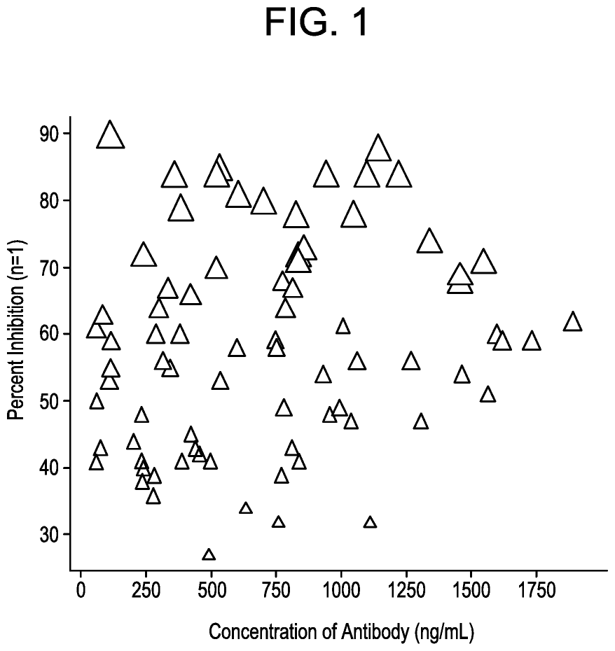

The amount of antigen-specific antibody in each well was quantitated and plotted against the neutralization data for that well to identify the highest potency wells (FIG. 1). The wells containing the highest potency antibodies are those with the best inhibition with the lowest concentration of antibody (upper left quadrant of the graph).

### Limiting Antigen (LA) Analysis of Anti-IL-13 Specific B Cell Culture Wells

The limited antigen analysis is a method that affinity ranks the antigen-specific antibodies prepared in B-cell culture supernatants relative to all other antigen-specific antibodies. In the presence of a very low coating of antigen, only the highest affinity antibodies should be able to bind to any detectable level at equilibrium. (See, e.g., PCT Publication WO03/048730A2, incorporated herein by reference).

Here, biotinylated IL-13 was bound to streptavidin plates at four concentrations (250 ng/mL; 125 ng/mL; 62 ng/mL; and 31 ng/mL) for 1 hour at room temperature on %-well culture plates. Each plate was washed 5 times with dH₂O and 45 μL of 1% milk in PBS with 0.05% sodium azide was added to the plate. This was followed by the addition of 5 μL of B cell supernatant to each well. After 18 hours at room temperature on a shaker, the plates were again washed 5 times with dH₂O. To each well was added 50 μL of Gt anti-Human (Fc)-HRP at 1 μg/mL. After 1 hour at room temperature, the plates were again washed 5 times with dH₂O and 50 μL of TMB substrate were added to each well. The reaction was stopped by the addition of 50 μL of 1M phosphoric acid to each well and the plates were read at wavelength 450 nm.

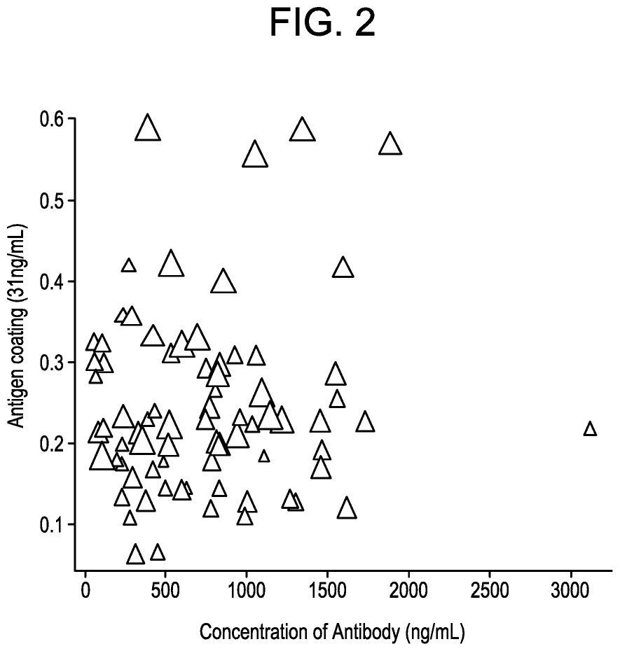

However, a number of wells including 2388A10 and 2357G11 were clearly superior as measured by OD at the lowest antigen coating, as illustrated in FIG. 2. The results presented in FIG. 2 demonstrate the ability of the different antibodies to bind at low concentration of antigen coating. The antibodies giving the highest OD signals have the highest affinity under the conditions of this assay. The remaining clones were further analyzed by combining the high antigen data which measures specific antibody concentration and the limited antigen output. In this way it was possible to compare the affinity of antibodies at different concentrations in B-cell culture supernatants. The wells containing the highest affinity antibodies are those with the highest ELISA OD in the context of lowest concentration of Ag-specific antibody.

Based on all of the screening data, the wells listed in Table 6 were identified for further analysis (plaque assay and micromanipulation, single cell PCR and recombinant expression).

Five wells were selected based on potency (inhibition/total specific Ab): 2372B8, 2383H5, 2398C5, 2401G12 and 2413G11. Three wells were selected based on affinity and inhibition: 2357G11, 2361G5 and 2384G12, and two wells were selected based on neutralization data alone: 2388A10 and 2407G11.

TABLE 6

| | | ELISA OD determined at different antigen coatings | | | |
|---|---|---|---|---|---|
| Plate | Well | 250 ng/ml | 125 ng/ml | 62 ng/ml | 31 ng/ml |
| 2357 | G11 | 2.582 | 1.553 | 1.066 | 0.59 |
| 2361 | G5 | 2.582 | 1.505 | 1.075 | 0.423 |
| 2372 | B8 | 1.616 | 0.79 | 0.506 | 0.234 |
| 2383 | H5 | 1.533 | 0.817 | 0.459 | 0.224 |
| 2398 | C5 | 1.187 | 0.694 | 0.425 | 0.186 |
| 2401 | G12 | 1.295 | 0.827 | 0.407 | 0.198 |
| 2413 | G11 | 1.274 | 0.783 | 0.449 | 0.203 |
| 2384 | G12 | 2.056 | 1.161 | 0.759 | 0.401 |
| 2388 | A10 | 2.637 | 1.76 | 1.152 | 0.558 |
| 2407 | G11 | 1.627 | 0.887 | 0.583 | 0.285 |

### IL-13-Specific Hemolytic Plaque Assay

Cells secreting IL-13-specific antibodies of interest were isolated utilizing an IL-13 specific hemolytic plaque assay generally as described in Babcook et al. (Proc. Natl. Acad. Sci. USA, 93:7843-7848 (1996), incorporated herein by reference). The cells that were isolated are identified in Table 7 below.

### Biotinylation of Sheep Red Blood Cells (SRBC)

SRBC were stored in RPMI media as a 25% stock. A 250 μl SRBC packed-cell pellet was obtained by aliquoting 1.0 ml of the stock into an eppendorf tube, spinning down the cells (pulse spin at 8000 rpm (6800 rcf) in microfuge) and removing the supernatant. The cells were then washed twice with 1 ml of PBS pH 8.6. The cell pellet was then re-suspended in 4.75 ml PBS at pH 8.6 in a 15 ml tube. In a separate 50 ml tube, 2.5 mg of Sulfo-NHS biotin was added to 45 ml of PBS at pH 8.6. Once the biotin had completely dissolved, the 5 ml of SRBCs were added and the tube rotated at RT for 1 hour. The SRBCs were centrifuged at 3000 g for 5 min, the supernatant drawn off and the SRBCs resuspended in 1 ml PBS at pH 7.4 in an Eppendorf tube. SRBCs were washed 3 times with 1 ml PBS at pH 7.4. The SRBCs were then resuspended in 4.75 ml immune cell media (RPMI 1640 with 10% FCS) in a 15 ml tube (5% B-SRBC stock). Stock was stored at 4° C. until needed.

### Streptavidin (SA) Coating of B-SRBC

One ml of the 5% B-SRBC stock was transferred into to a fresh eppendorf tube. The B-SRBCs were pelleted, the supernatant drawn off, the pellet re-suspended in 1.0 ml PBS at pH 7.4, and the centrifugation repeated. The wash cycle was repeated 2 times, and then the B-SRBC pellet was resuspended in 1.0 ml of PBS at pH 7.4 to give a final concentration of 5% (v/v). 10 μL of a 10 mg/ml streptavidin (CalBiochem, San Diego, Calif.) stock solution was added and the tube mixed and rotated at RT for 20 min. The washing steps were repeated and the SA-SRBC were re-suspended in lml PBS pH 7.4 (5% (v/v)).

### Human IL-13 Coating of SA-SRBC

The SA-SRBC were coated with photobiotinylated-Human IL-13-RbFc fusion at 100 ug/ml, then mixed and rotated at RT for 20 min. The SRBC were washed twice with 1.0 ml of PBS at pH 7.4 as above. The IL-13-coated SRBC were re-suspended in RPMI (+10% FCS) to a final concentration of 5% (v/v).

### Determination of the Quality of IL-13-SRBC by Immunofluorescence (IF)

Approximately 10 μl of 5% SA-SRBC and 10 μl of 5% IL-13-coated SRBC were each added to separate fresh 1.5 ml eppendorf tube containing 40 μl of PBS. A control human anti-IL-13 antibody was added to each sample of SRBCs at 45 μg/ml. The tubes were rotated at RT for 20 min, and the cells were then washed three times with 100 ul of PBS. The cells were re-suspended in 50 μl of PBS and incubated with 20 sg/mL Gt-anti Human IgG Fc antibody conjugated to Alexa488 (Molecular Probes, Eugene, Oreg.). The tubes were rotated at RT for 20 min, and then washed with 100 μl PBS and the cells re-suspended in 10 0 PBS. 10 0 of the stained cells were spotted onto a clean glass microscope slide, covered with a glass cover slip, observed under fluorescent light, and scored on an arbitrary scale of 0-4.

### Preparation of Plasma Cells

The contents of a single B cell culture well previously identified by the various assays described above as containing a B cell clone secreting the immunoglobulin of interest were harvested. Using a 100-1000 μL pipetteman, the contents of the well were recovered by adding 37C RPMI (+10% FCS). The cells were re-suspended by pipetting and then transferred to a fresh 1.5 ml Eppendorf tube (final vol. approx 700-1000 μl). The cells were centrifuged in a microfuge at 2500 rpm for 1 minute at room temperature. The tube was then rotated 180 degrees and spun again for 1 minute at 2500 rpm. The freeze media was drawn off and the immune cells resuspended in 100 μL RPMI (10% FCS), then centrifuged. This washing with RPMI (+10% FCS) was repeated and the cells re-suspended in 75 μL RPMI (+10% FCS) and stored on ice until ready to use.

### Plaque Assay

To a 75 μL sample of cells was added 75 uL each of IL-13-coated SRBC (5% (v/v) stock, diluted as necessary if the SRBC lawn was too thick), 4× guinea pig complement (Sigma, Oakville, ON) stock prepared in RPMI (+10% FCS), and 4× enhancing sera stock (1:900 in RPMI (+10% FCS)). The mixture (3-5 μL) was spotted onto TC plate lids (BD Biosciences, San Jose, Calif.) and the spots covered with undiluted paraffin oil. The slides were incubated at 37° C. for a minimum of 1 hour.

TABLE 7

| Plate | Well | Single Cell (SC) Numbers |
|---|---|---|
| 2407 | G11 | SC-IL-13-557-576 |
| 2388 | A10 | SC-IL-13-577-596 |
| 2401 | G12 | SC-IL-13-597-616 |
| 2372 | B8 | SC-IL-13-617-636 |
| 2413 | G11 | SC-IL-13-637-657 |
| 2398 | C5 | SC-IL-13-658-670 |
| 2383 | H5 | SC-IL-13-671-690 |
| 2384 | G12 | SC-IL-13-691-710 |
| 2357 | G11 | SC-IL-13-711-730 |
| 2361 | G5 | SC-IL-13-731-750 |

### Cloning and Expression

After isolation of the single plasma cells, mRNA was extracted and reverse transcriptase PCR was conducted to generate cDNA encoding the variable heavy and light chains of the antibody secreted by each cell. The human variable heavy chain region was cloned into an IgG2 expression vector. This vector was generated by cloning the constant domain of human IgG2 into the multiple cloning site of pcDNA3.1+/Hygro (Invitrogen, Burlington, ON). The human variable light chain region was cloned into an IgK or IgL expression vector. These vectors were generated by cloning the constant domain of human IgK or human IgL into the multiple cloning site of pcDNA3.1+/Neo (Invitrogen, Burlington, ON).

The heavy chain and the light chain expression vectors were then co-transfected using lipofectamine into a 60 mm dish of 70% confluent human embryonal kidney (HEK) 293 cells. The transfected cells secreted a recombinant antibody with the identical specificity as the original plasma cell for 24-72 hours. 3 mL of supernatant was harvested from the HEK 293 cells and the secretion of an intact antibody was demonstrated with a sandwich ELISA to specifically detect human IgG. Specificity was confirmed through binding of the recombinant antibody to IL-13 using ELISA.

The secretion ELISA tests were performed as follows. Control plates were coated with 2 mg/mL goat anti-human IgG H+L overnight as for binding plates, IL-13 was coated onto Costar Labcoat Universal Binding Polystyrene 96 well plates and held overnight at 4° C. The plates were washed five times with dH₂O. Recombinant antibodies were titrated 1:2 for 7 wells from the undiluted lipofection supernatant. The plates were washed five times with dH₂O. A goat anti-human IgG Fc-specific HRP-conjugated antibody was added at a final concentration of 1 μg/mL for 1 hour at RT for the secretion and the two binding assays. The plates were washed five times with dH₂O. The plates were developed with the addition of TMB for 30 minutes and the ELISA was stopped by the addition of 1 M phosphoric acid. Each ELISA plate was analyzed to determine the optical density of each well at 450 nm.

### Purification of Recombinant Anti-IL-13 Antibodies

For larger scale production, heavy and light chain expression vectors (2.5 μg of each chain/dish) were lipofected into HEK293 cells in ten 100 mm dishes that were 70% confluent. The transfected cells were incubated at 37° C. for 4 days, at which time the supernatant (6 mL) was harvested and replaced with 6 mL of fresh media. At day 7, the supernatant was removed and pooled with the initial harvest (120 mL total from 10 plates).

Each antibody was purified from the supernatant using Protein-A Sepharose (Amersham Biosciences, Piscataway, N.J.) affinity chromatography (1 mL). The antibodies were eluted from the Protein-A column with 500 mL of 0.1 M Glycine (pH 2.5). The eluates were dialyzed in PBS (pH 7.4) and filter sterilized. The antibodies were analyzed by non-reducing SDS-PAGE to assess purity and yield. Concentration was also measured by UV analysis at OD 280.

### Example 2: Recombinant Antibody Characterization

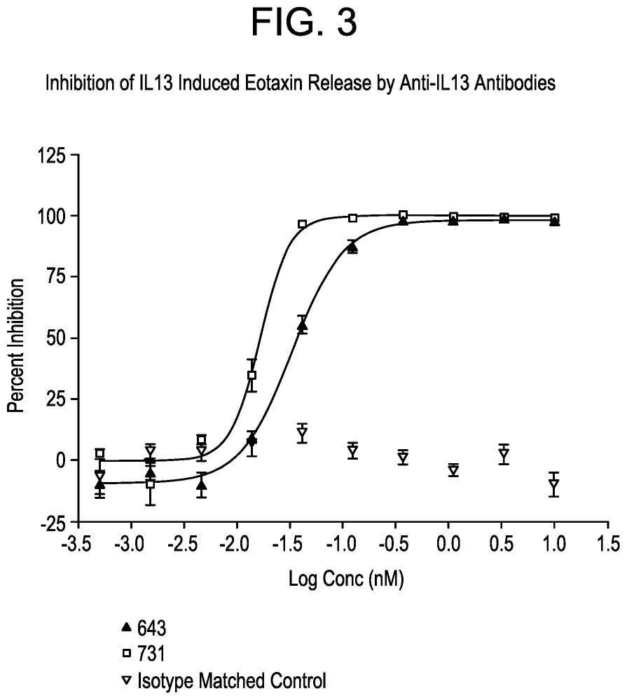

Recombinant antibodies were analyzed for potency in the Eotaxin-1 assay as described above. The results are presented in Table 8 below. Also included are the measured IC50's in this assay for murine IL-13 receptor α2/FC and human IL-13 receptor α2/Fc. FIG. 3 shows the percent inhibition of IL-13 induced eotaxin release by recombinant antibodies 643 and 731 compared to an isotype matched control, e.g., an irrelevant IgG2 monoclonal antibody.

TABLE 8

| | IC50 (pM) | | | | |
|---|---|---|---|---|---|
| | | | | | Standard |
| mAb ID | n = 1 | n = 2 | n = 3 | Average | Dev |
| 731 | 11 | 19 | 17 | 16 | 4 |
| 713 | 21 | 21 | 19 | 21 | 1 |
| mIL-13Ralpha2/Fc | 29 | 39 | 29 | 32 | 6 |
| 643 | 44 | 28 | 33 | 35 | 8 |
| 623 | 31 | 40 | 35 | 36 | 4 |
| 693 | 38 | 69 | 53 | 54 | 16 |
| 602 | 80 | 53 | ND | 66 | NA |
| 353 | 99 | 123 | 80 | 101 | 22 |
| hIL-13Ralpha2/Fc | 128 | 147 | 119 | 131 | 14 |
| 785 | 223 | 144 | 160 | 176 | 42 |
| 11.18.3 | 213 | 304 | 217 | 245 | 51 |
| 157 | 260 | 207 | 306 | 258 | 50 |
| 176 | 233 | ND | ND | 233 | NA |
| 183 | 1040 | 1842 | ND | 1441 | NA |
| 264 | 293 | 313 | 284 | 297 | 15 |
| 243 | 253 | ND | ND | 253 | NA |
| 356 | 1087 | 913 | ND | 1000 | NA |

### BiaCore Affinity

Affinity to human IL-13 (R&D) was investigated by BiaCore assay for six of the antibodies (602, 623, 643, 693rep1, 693rep2 and 7310). First, two high-density goat α-human antibody surfaces were prepared on a CM5 Biacore chip using routine amine coupling for the capture of the mAbs three at a time. All mAbs were diluted to ˜5 μg/Ml using HBS-P running buffer containing 100 μg/ml BSA. Each purified mAb was captured for one minute on a different flow cell surface for every IL-13 injection cycle using a Biacore 2000 instrument.

IL-13 (R&D) was injected using the KINJECT command at concentrations of 100.9, 50.4, 25.2, 12.6, 6.30, 3.15, 1.58 and 0.788 nM for mAbs 693, 713 and 731 and 25.2, 12.6, 6.30, 3.15, 1.58, 0.788, and 0.394 nM for mAbs 602, 623, and 643, over all surfaces for 1.5 min., followed by a twenty minute dissociation. The IL-13 samples were prepared in HBS-P running buffer containing 100 μg/ml BSA. All samples were randomly injected in duplicate with several mAb capture/buffer KINJECT cycles interspersed for double referencing.

The high-density goat α-human antibody surfaces were regenerated with a 12-second pulse of 1/100 diluted concentrated phosphoric acid (146 mM, pH 1.5) after each cycle. mAb 693 was run twice because there was an extra flow cell available on the instrument during the last series of medium resolution experiments.

The data was fit to a 1:1 interaction model with a term for mass transport using CLAMP. The data for the six antibodies are shown in Table 9.

TABLE 9

| Antibody | kₐ (M⁻¹ s⁻¹) | k_{d} (s⁻¹) | K_{D} (pM) |
|---|---|---|---|
| 602 | 3.0 × 10⁶ | 5.1 × 10⁻⁴ | 172 |
| 623 | 4.9 × 10⁶ | 2.5 × 10⁻⁴ | 52 |
| 643 | 4.4 × 10⁶ | 2.9 × 10⁻⁴ | 66 |
| 693 rep 1 | 2.0 × 10⁶ | 3.8 × 10⁻⁴ | 189 |
| 693 rep 2 | 2.5 × 10⁶ | 2.7 × 10⁻⁴ | 109 |
| 713 | 2.9 × 10⁶ | 3.4 × 10⁻⁵ | 12 |
| 731 | 3.9 × 10⁶ | 3.5 × 10⁻⁵ | 9 |

### Kinetic Analysis

Kinetic measurements of several of the antibodies were evaluated using the KinExA® method. This method involves solution-based determination of formal affinity measurements at equilibrium.

One hundred μg of each mAb was coupled to CNBr-activated Sepharose 4B or Azlactone beads. The remaining active groups on the beads were blocked as recommended by the manufacturer. The beads were then blocked with 10 mg/ml BSA in 1 M Tris and stored in the blocking solution. For some experiments the mAb was directly absorption coated to PMMA beads as recommended by the manufacturer and blocked with 10 mg/ml BSA in PBS and stored in the blocking solution.

KinExA experiments were performed using an automated flow immunoassay system, KinExA 3000, in which beads coupled with the relevant mAbs served as the solid phase. Briefly, a constant amount of native human or macaque monkey IL-13 (10-650 pM), prepared by purifying and stimulating PBMCs according to standard protocols, was incubated with titrating concentrations of anti-h-IL-13 mAbs starting at 25 nM in sample buffer (PBS with 0.1% BSA to reduce nonspecific binding). Antigen/antibody complexes were incubated at RT for 48 hrs to 168 hrs to allow equilibrium to be reached. The mixture was drawn through the corresponding antibody-coupled beads to accumulate unbound antigen. The volumes and flow rates of the mixture were varied depending upon the specific signal obtained in each experiment.

The captured IL-13 was detected using solutions containing a secondary Ab (either a polyclonal anti-IL-13 Ab or a monoclonal Ab that binds to another epitope) and Cy5-conjugated anti-species Ig to the secondary antibody in sample buffer. In some cases the bead bound IL-13 was detected using a mixture of SA-Cy5 and a biotinylated antibody that binds to an epitope other than that bound by the bead immobilized Ab.

The concentrations, volumes, and flow rates of the secondary antibody solutions were varied to optimize the signal to noise ratio in each experiment. The bound signals were converted into relative values as a proportion of control in the absence of hIL-13. Three replicates of each sample were measured for all equilibrium experiments. The equilibrium dissociation constant (K_{D}) was obtained from nonlinear regression analysis of the data using a one-site homogeneous binding model contained within the KinExA software. The software calculates the K_{D} and determines the 95% confidence interval by fitting the data points to a theoretical K_{D} curve. The 95% confidence interval is given as K_{D} low and K_{D} high. The affinities are summarized in Tables 10 for native human IL-13 and 11 for native macaque IL-13.

TABLE 10

| Antibody | K_{D} | K_{D}low | K_{D}high |
|---|---|---|---|
| 623 | 24 pM | 6.6 pM | 60 pM |
| 643 | 13 pM | 6.2 pM | 25 pM |
| 713 | 3.6 pM | 1.1 pM | 7.3 pM |
| 731 | 8.9 pM | 6.2 pM | 12 pM |

TABLE 11

| Antibody | K_{D} | K_{D}low | K_{D}high |
|---|---|---|---|
| 623 | 37 pM | 18 pM | 64 pM |
| 731 | 1.6 nM | 880 pM | 2.2 nM |

The association rate constant was investigated using KinExA for two of the antibodies, 623 and 731. The same IL-13 coupled beads were used as the probe and the “direct” or “injection” methods were used. These methods are identical to the KinExA equilibrium assays with respect to antigen capture, antigen concentration and antigen detection. In the direct method, the antigen and antibody are mixed in advance and then run on the KinExA. In the injection method, the antibody and a titration of antigen are mixed together for a set time before reading. Briefly, hIL-13 was mixed with an amount of mAb that would bind approximately 80% of the antigen based on the equilibrium experiments. The free antigen present in the sample was probed repeatedly, pre-equilibrium. Since the binding signals are proportional to the concentration of free antigen in the solution, the signals decreased over time until the solution reached equilibrium. The volumes and flow rates of the antigen-mAb mixtures and the Cy5-labeled secondary antibody were varied depending upon the mAb tested. Data was analyzed utilizing the KinExA analysis software. This software graphically represents the decrease in binding signals over time, and fits the collected data points to an exact solution of the kinetic differential equations for binding. From this curve, an optimal solution for the kₒₙ was determined (Table 12). The k_{off} was indirectly calculated from solutions for the kₒₙ and K_{D}.

TABLE 12

| | | KD | | kₒₙ bounds | | | |
|---|---|---|---|---|---|---|---|
| Antibody | Method | (pM) | kₒₙ (M⁻¹ s⁻¹) | kₒₙ High | kₒₙ Low | k_{off} (s⁻¹) | % Error |
| 623 | Kinetic | 24 | 1.1E+07 | 1.4E+07 | 5.1E+06 | 2.7E−04 | 1.37 |
| | Direct | | | | | | |
| 623 | Kinetic | 24 | 1.5E+07 | 2.1E+07 | 1.1E+07 | 3.6E−04 | 5.46 |
| | inject | | | | | | |
| 731 | Kinetic | 8.9 | 4.7E+06 | 6.3E+06 | 3.4E+06 | 4.2E−05 | 4.96 |
| | inject | | | | | | |

### Binding to the IL-13 Variant Protein

The ability of antibodies 623 and 731 to bind to an IL-13 variant protein in which the wildtype arginine 110 is replaced with glutamine (IL-13Q110R) was investigated.

Briefly, plates were coated in IL-13RbFc (50 μL of 2.5 μg/mL) by incubation in 1×PBS (pH7.4) and 0.05% azide overnight at 4′C. The plates were then washed with 1×PBS and blocked for 30 minutes with 100 μL of 1% no fat skim milk/1×PBS at room temperature.

IL-13 or IL-13Q110R was pre-incubated with anti-IL-13 antibodies for 1 hr at room temperature. Titrated IL-13 vertically from 2000 ng/ml with final volume of 30 μl/well. 30 μl of mAb was added per well at 40 ng/ml (sc731, 623) and 80 ng/ml (sc693), resulting in a final concentration of IL-13 at the first point in the titration of 1000 ng/ml, a final concentration of antibodies 623 and 731 at the first point in the titration of 20 ng/ml and final concentration of antibody 693 at the first point in the titration of 40 ng/ml.

After pre-incubation, 50 μl/well was transferred from the pre-incubation solution to a plate pre-coated with IL-13RbFc and incubated for 30 minutes at room temperature. Plates were washed and rabbit anti Hu IgG Fc HRP was added at a concentration of 200 ng/ml. Following a further 30 minutes incubation and subsequent wash, TMB was added and incubated for an additional 30 minutes. Reactions were stopped with 1N HCL and plates were read as soon as possible on a Powerwave X340 96 well microplate reader (Biotek).

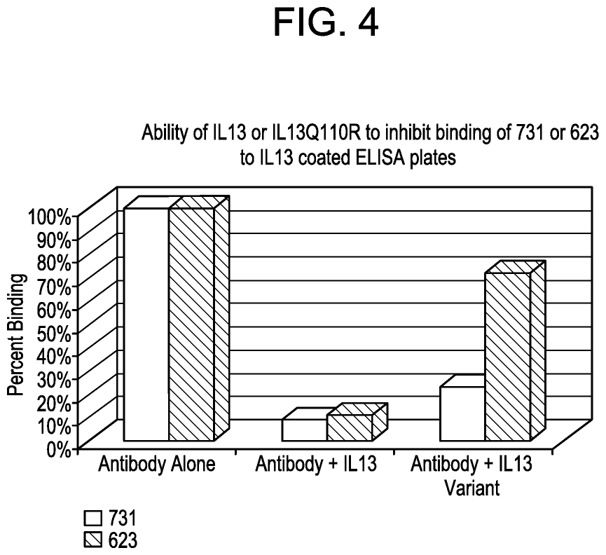

As can be seen in FIG. 4, pre-incubation with IL-13 inhibits binding of both antibodies 623 and 731 to IL-13 coated ELISA plates, while pre-incubation with IL-13 variant IL-13Q110R inhibits binding of 731 to a much greater extent than binding of 623.

### Receptor Chain Competition

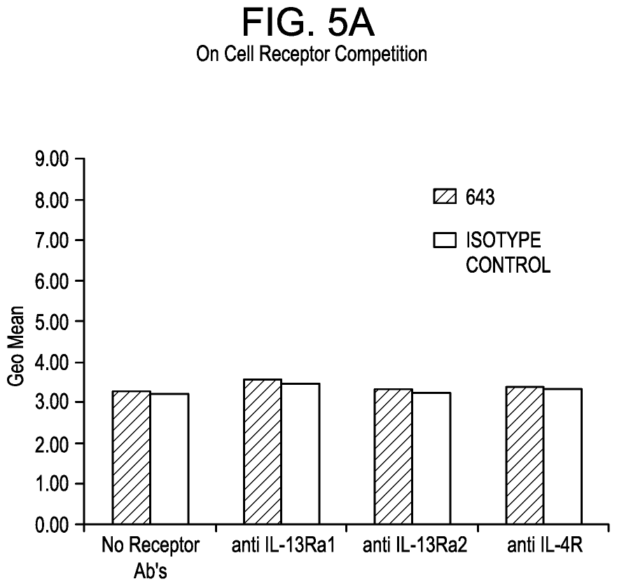

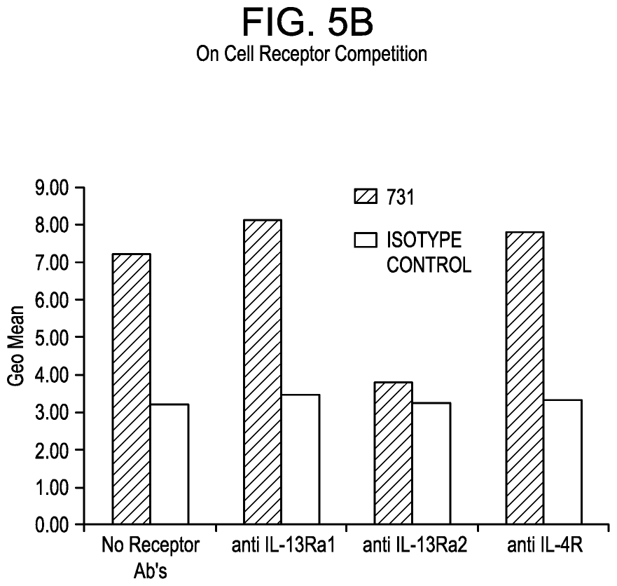

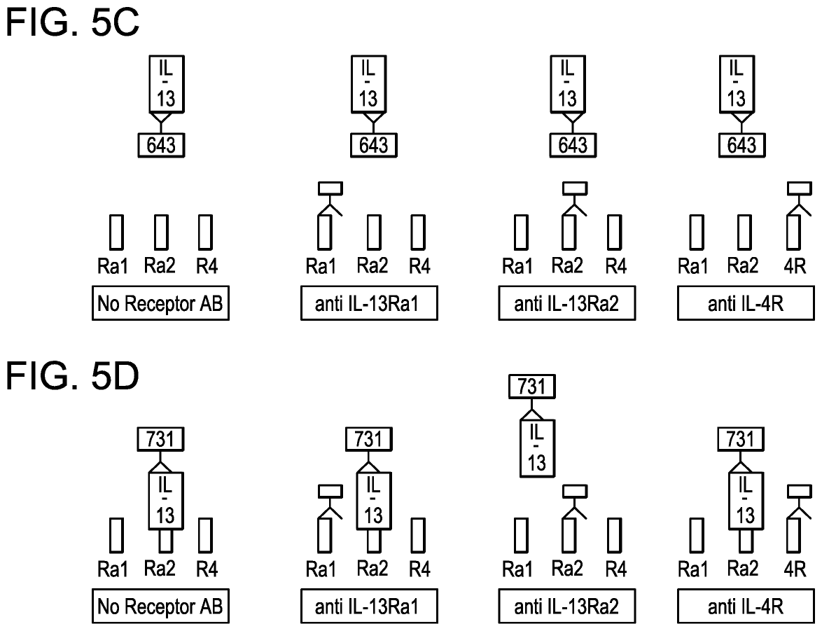

The ability of anti-IL-13 antibodies to block IL-13 binding to the receptors IL-13Rα1 and IL-13Rα2 was investigated. Samples were analyzed using the flow cytometer. The results are presented in FIG. 5A and FIG. 5B. The data demonstrated the ability of Ab 643 (FIG. 5A) and of Ab 731 (FIG. 5B) or an isotype control antibody to bind to IL-13 and the receptors involved in the binding process. The particular receptor (e.g., IL-13Ra2, IL-13Ra1, or IL-4R) that was binding IL-13 and allowing the antibody to interact with the cells was determined using neutralizing antibodies against all possible IL-13 receptors expressed on HDFa cells. A summary of the various experiments and predicted results is displayed in FIG. 5C and FIG. 5D (adjust figure legends if this change is accepted).

Briefly, HDFa cells were resuspended in FACS buffer to yield about 200 000 cells/well/100 μL and 100 μL of cells were aliquoted into 96-well VEE bottom plates. Neutralizing anti-receptor antibodies (anti human IL-13Ra1 (R&D Systems), anti human IL-13Ra2 (R&D Systems) or anti human IL-4R (R&D Systems)) were diluted in FACS buffer at twice the final concentration (10 μg/mL FINAL). Anti-IL-13 and Control Ab's were also diluted in FACS buffer at 2× final concentration (1 μg/mL), as was IL-13 (human R&D; 10 ng/mL FINAL).

A VEE bottom plate of HDFa cells was centrifuged at 180×g for 7 min and the supernatant removed by inversion (PLATE #1). Cells were resuspended in 50 μL FACS buffer and an additional 50 μL of anti human IL-13Ra1, anti human IL-13Ra2, anti human IL-4R or FACS buffer (No Receptor Ab Control) was added to appropriate wells. The cells and antibodies were then incubated on ICE for about 1.5 hrs.

A second VEE bottom plate was used for Ab/IL-13 pre-incubation (PLATE #2). 60 μL of the test antibody was aliquoted into aVEE bottom plate. 60 μL of IL-13 added to appropriate wells and the mixture was incubated on ice for about 1.5 hrs.

After the incubation HDFa cells were centrifuged at 180×g for 7 min and the supernatant was removed by inversion. The cells in PLATE #1 were resuspended in 100 μL FACS buffer or 100 μL of Ab/IL-13 and incubated for a further 1.5 hrs.

Following the second incubation the cells were centrifuged, washed 1× with FACS buffer and 100 μL of FACS buffer, 7AAD or 2 μg/mL goat anti Hu IgG-Fc-Cy5 was added to appropriate wells.

The cells and secondary antibody were incubated on ice for 20 minutes, followed by a wash with FACS buffer. Cells were then resuspended in 100 μL FACS buffer and aliquoted into pre-labeled FACS tubes containing 300 μL cold FACS buffer.

Samples were analyzed using the flow cytometer. The results are presented in FIG. 5A and FIG. 5B. A summary of the above protocol and predicted results for each ofthe antibodies is shown in FIG. 5C and FIG. 5D. As shown by FIG. 5A, IL-13 does not bind to HDFa cells in the presence of Ab 623. It appears that Ab 623 prevents IL-13 from binding to its receptors on HDFa cells, as shown in each of the panels of FIG. 5C. As can be seen in FIG. 5B, this is not the case for Ab 731. IL-13 allows Ab731 to bind to HDFa cells. This binding is not blocked by Abs against IL-13Ralpha1 or IL-4R but is blocked by antibodies against IL-13Ralpha2, indicating that Ab 731 prevents IL-13 from binding to IL-13Ralpha1 or IL-4R but not to IL-13Ralpha2, as displayed in FIG. 5D.

The amount of IL-13 Ra1, IL-13 Ra2 and IL-4R surface expression on HDFa cells was determined by FACS analysis using anti Receptor antibodies. HDFa cells prepared as described above were incubated with anti-receptor antibodies at a concentration of 5 μg/ml on ice for 1 hr. Cells were washed with FACS buffer and incubated with Cy5 secondary (anti-hum) antibody at 2 μg/ml. on ice for 30 min. After washing, samples were analyzed by flow cytometry. The results are presented in Table 13 below.

TABLE 13

| Antibody Target | FACS Geometric Mean Average |
|---|---|
| IL13 Receptor Alpha 1 | 8.39 |
| IL13 Receptor Alpha 2 | 9.4 |
| IL4 Receptor Alpha 1 | 9.15 |
| Negative Control | 3.8 |

### Epitope Mapping

The epitopes for the antibody-IL-13 complexes were analyzed by three methods, 1) SELDI, 2) Screening of Random peptide phage display libraries, and 3) expression of Chimeric Human/Mouse IL-13 molecules. These three techniques combined with knowledge of the structure of IL-13 produced a coherent view of the relative binding sites and antigenic regions of these mAbs. This has permitted the identification of functional epitopes, particularly for the regions involved in binding to the signaling receptor.

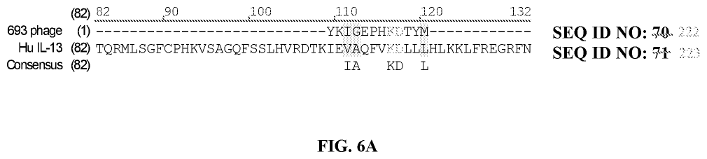

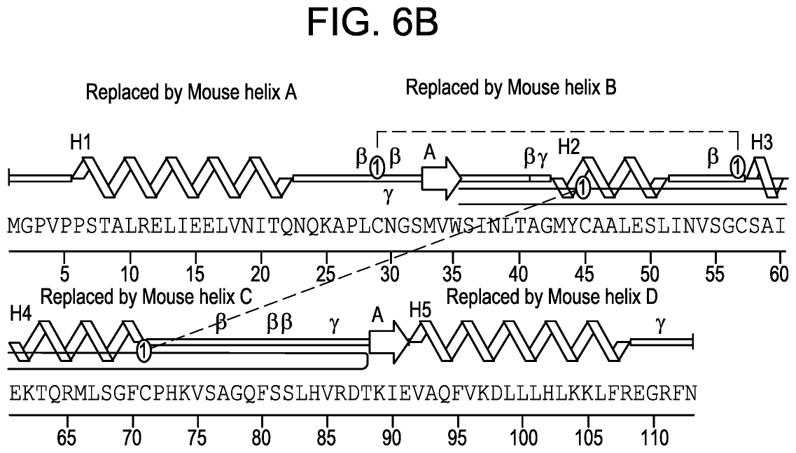

As an initial examination, dot blot analysis of mAb binding to IL-13 purified protein revealed which antibodies bound to which form (linear or conformational) of the epitope. mAbs 693 and 785 bound to the reduced denatured antigen, the linear epitope. mAbs 602,623, 643, and 713, bound to the non-reduced (conformational epitope) IL-13 but not to the reduced denatured antigen. mAb 763 displayed no binding. Following this, the linear epitopes were mapped using random peptide phage display library. After two rounds of panning mAb 693 against a 12-mer random peptide library expressed on phage, a single specific binder was sequenced and aligned to residues 109-120 (Helix D) of IL-13. (FIG. 6A). IL-13 antibodies were grouped in 3 different bins, although bins do not always correlate with epitopes determined by other means. One antibody from each bin was picked for mapping by SELDI. Table 14 demonstrates the binning results of the IL-13 antibodies.

TABLE 14

| Mab | VH | VL | Bin |
|---|---|---|---|
| 353 | VH4-59/D2-21/JH3b | A30/JK3 | 1 |
| 713 | VH3-23/D6-19/JH6b | V2-1/JL1 | 1 |
| 731 | VH3-23/D6-19/JH6b | V2-1/JL1 | 1 |
| 602 | VH3-15/D1-26/JH6b | V2-7/JL3 | 2 |
| 623 | VH3-15/D1-26/JH6b | V2-7/JL3 | 2 |
| 643 | VH3-15/D1-26/JH6b | V2-7/JL3 | 2 |
| 693 | VH4-4/D5-5/JH6B | V2-14/JL2 | 3 |

### Mapping of Epitopes Using SELDI

The antibody-antigen complex was digested with a high concentration of Lys-C and Asp-N. The epitope was then determined by SELDI and identified by the mass of the fragment Table 15 displays the predicted masses for the peptides digested with endoproteinase Lys-C.

TABLE 15

| | | | | SEQ |
|---|---|---|---|---|
| | | Mis. | | ID |
| Mass | Position | Cut | Peptide Sequence | NO: |
| 9442.7 | 21-108 | 3 | GPVPPSTALRELIEELVNIT | 14 |
| | | | QNQKAPLCNGSMVWSINLTA | |
| | | | GMYCAALES | |
| | | | LINVSGCSAIEKTQRMLSGF | |
| | | | CPHKVSAGQFSSLHVRDTK | |
| 7829.9 | 21-93 | 2 | GPVPPSTALRELIEELVNIT | 15 |
| | | | QNQKAPLCNGSMVWSINLTA | |
| | | | GMYCAALESLINVSGCSAIE | |
| | | | KTQRMLSGFCPHK | |
| 7729.8 | 45-116 | 3 | APLCNGSMVWSINLTAGMYC | 16 |
| | | | AALESLINVSGCSAIEKTQR | |
| | | | MLSGFCPHKVSAGQFSSLHV | |
| | | | RDTKIEVAQFVK | |
| 6815.3 | 45-108 | 2 | APLCNGSMVWSINLTAGMYC | 17 |
| | | | AALESLINVSGCSAIEKTQR | |
| | | | MLSGFCPHKVSAGQFSSLHV | |
| | | | RDTK | |

The masses identified following cleavage were 6842.8 (for peptide fragment 45-108), 7733.7 (for peptide fragment 45-116), and 9461.4 (for peptide fragment 21-108). Thus, the binding site for mAb 713 was determined to be within residues 45-108 of IL-13.

### Peptide Array for Mapping Conformational Epitopes

A peptide array of 101, 12-mer peptides, spanning residues 21-132 of the IL-13 sequence was generated (SIGMA-Genosys). Each consecutive peptide was offset by one amino acid from the previous one, yielding a nested, overlapping library. The array was probed with mAb 713 and binding of mAb713 to the peptides was detected by incubating the PVDF membranes with HRP-conjugated secondary antibody followed by enhanced chemiluminescence. Two consecutive spots, corresponding to amino acids 70 to 80 of IL-13 and three consecutive spots, corresponding to amino acids 83 to 92 or IL-13 were observed.

### Epitope Mapping Using Mouse IL-13 Chimeric Molecules

Mouse sequences of Helix A, Helix B, Helix C, and Helix D were shuffled with human sequences generating four new mouse chimeras. A representation of the location of the helices is shown in FIG. 6B. None of the mAbs bound to the mouse IL-13. The four chimeras are as follows:

(SEQ ID NO: 18)
MHPLLNPLLLALGLMALLLTTVIALTCLGGFASPGPVPRSVSLPLTLKEL
IEELVNITQNQKAPLCNGSMVWSINLTAGMYCAALESLINVSGCSAIEKT
QRMLSGFCPHKVSAGQFSSLHVRDTKIEVAQFVKDLLLHLKKLFREGQF
N;
(SEQ ID NO: 19)
MHPLLNPLLLALGLMALLLTTVIALTCLGGFASPGPVPPSTALRELIEEL
VNITQNQKAPLCNGSMVWSINLTAGGFCVALDSLTNVSGCSAIEKTQRML
SGFCPHKVSAGQFSSLHVRDTKIEVAQFVKDLLLHLKKLFREGQFN;
(SEQ ID NO: 20)
MHPLLNPLLLALGLMALLLTTVIALTCLGGFASPGPVPPSTALRELIEEL
VNITQNQKAPLCNGSMVWSINLTAGMYCAALESLINVSGCSAIYRTQRIL
HGLCPHKVSAGQFSSLHVRDTKIEVAQFVKDLLLHLKKLFREGQFN;
(SEQ ID NO: 21)
MHPLLNPLLLALGLMALLLTTVIALTCLGGFASPGPVPPSTALRELIEEL
VNITQNQKAPLCNGSMVWSINLTAGMYCAALESLINVSGCSAIEKTQRML
SGFCPHKVSAGQFSSLHVRDTKIEVAHFITKLLSYTKQLFRHGQQFN.

The chimeras were then expressed and secreted IL-13 chimeric proteins were detected in an ELISA assay. The results are summarized in Table 16, the “*” denotes that the binding was weak in the sandwich ELISA.

TABLE 16

| | Hu IL- | Mo | Mo | Mo | Mo | | |
|---|---|---|---|---|---|---|---|
| mAb | 13 | HelixA | HelixB | HelixC | HelixD | Epitope | Bin |
| 693 | Yes | Yes | Yes | Yes | No | HelixD | 3 |
| 785 | Yes | Yes | Yes | Yes | No | HelixD | |
| 713* | Yes | Yes | Yes | No | Yes | HelixC | 1 |
| 731* | Yes | Yes | Yes | No | Yes | HelixC | 1 |
| 602 | Yes | Yes | Yes | Yes | Yes | | 2 |
| 623 | Yes | Yes | Yes | Yes | Yes | | 2 |
| 643 | Yes | Yes | Yes | Yes | Yes | | 2 |

The results of the above three studies of the epitopes of IL-13 are summarized in Table 17.

TABLE 17

| Mab | Phage Display | SELDI | Spots | Chimera | Bin |
|---|---|---|---|---|---|
| 3.1.2.4 | 21-33 | | | HelixA | |
| 693 | 109-121 | | | HelixD | 3 |
| 785 | 111-128 | | | HelixD | |
| 713 | | 45-108 | 70-80 and 83-92 | HelixC | 1 |
| 731 | | | | HelixC | 1 |
| 602 | | | | | 2 |
| 623 | | | | | 2 |
| 643 | | | | | 2 |

Thus, it appears that a number of different possible epitope positions are used by the various antibodies disclosed herein.

### Antibody Binning Analysis

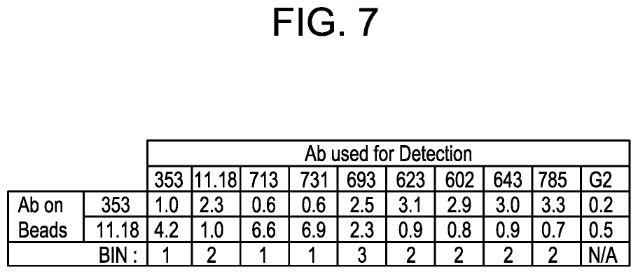

Anti-IL-13 antibodies were grouped in three different bins by measuring the ability of two antibodies to bind to antigen at the same time (one antibody capturing the antigen on a bead and the other antibody used for detection). The signal on the beads in the absence of antigen was subtracted from the signal obtained in the presence of antigen. The signal of each detection antibody was divided by the signal of the capture antibody to determine the fold increase in binding as shown in FIG. 7. The antibodies were then binned based on similar binding patterns on the capture antibodies. The data identified the presence of three bins of antibody binding for the nine detection antibodies tested (FIG. 7).

Briefly, mouse anti-human IgG1,2,3,4 (BD Pharmingen 555784) conjugated beads were added to capture antibody (353 & 11.18; 5 ug/mL) in individual darkened eppendorf tubes. The tubes were rotated in the dark at 4° overnight. Beads were aliquoted to each well of a filter plate (2500 of each bead/well) and washed.

IL-13-RbIg (5 μg/ml) and controls (media only) were added to the filter plate 60 μl/well, which was then incubated in the dark at room temperature for 1 hour on a shaker and subsequently washed 2 times.

Secondary antibodies diluted in media at 60 μl/well (1 antibody per well) were added. The antibodies were used at the following concentrations (353B—5 g/ml; 11.18.31—5 μg/ml; 713—0.56 μg/ml; 731—1.28 μg/ml; 693—2.7 μg/ml; 623—5.7 μg/ml; 602—11 μg/ml; 643—4.3 μg/ml; 785—5.5 μg/ml; 763—5.7 μg/ml; G2 control—5 μg/ml). Plates were then incubated for two hours at room temperature and washed.

Biotinylated Mo-anti-HuIg G1,2,3,4 (BD Pharmingen #555785) diluted in medium at 5 μg/ml was added to each well (60 μl/well) and the plates were incubated in the dark for 1 hour on a shaker at room temperature. After washing 60 μl/well Streptavidin-PE (5 ug/mL; Pharm #554061) diluted in medium was added. Plates were incubated in the dark for 20 min on the shaker at room temperature and washed 2 times.

Each well was resuspended in 80 μl storage/blocking buffer (PBS, 10 mg/ml BSA, 0.05% w/v sodium azide) by carefully pipette up and down several times to resuspend beads. Each well was analyzed by reading on Luminex with the gate set between 8,400 and 14,500.

The Luminex platform is a fluorescence bead based technology which enables one to run multiple assays at once. The Luminex reader is able to ascertain positive signaling events on different coded microspheres. This allows one to coat each bead separately, then mix the differentially coated microspheres together and then in one step assay antibody binding to each of the different microspheres. For isotyping antibodies, microspheres were coated in such a manner in that each bead was able to specifically bind a particular heavy chain or light chain isotype. The microspheres were then mixed together and hybridoma supernatant for each antibody was added. After a 20 minute incubation, the microspheres were washed, and the bound antibody was detected using a fluorescently labeled secondary antibody. The microspheres were then read using the Luminex reader.

### Example 3: Pre-Clinical In Vivo Data

### Humanized IL-13 Mice

Humanized IL-13 mice, in which the gene encoding murine IL-13 was disrupted by the insertion of a cDNA encoding human IL-13, were generated at Lexicon (The Woodlands, Tex.). Mice were backcrossed onto the A/J strain to ensure that the mice were susceptible to allergen-induced airway hyper-reactivity as previously described (Ewert et al., (2000) Am. J. Respir. Cell. Mol. Biol.).

To demonstrate that humanized IL-13 mice produce only human IL-13 and no murine IL-13, cytokine production from OVA-specific CD4⁺ T cells derived from humanized IL-13 mice (6-8 wk of age) were compared with CD4⁺ T cells derived from WT mice. Mice were sensitized by i.p. injection with 50 μg OVA/1 mg Imject Alum (Pierce, Rockford, Ill.) in 0.9% sterile saline or with PBS (3 mice per treatment). Seven days after sensitization, mice were sacrificed, and single-cell suspensions of the spleens were prepared. Erythrocytes were lysed, and the washed splenocytes were resuspended at 5×10⁶ cells/ml in complete medium consisting of HL-1 (BioWhittaker, Walkersville, Md.) with 10% heat-inactivated FCS, 2 mM L-glutamine, and 50 mg/L neomycin sulfate. Splenocytes were then cultured for 4 days at 37° C. in the presence of 200 μg/ml OVA to generate Ag-reactive CD4⁺ T cells. CD4⁺ T cells (5×10⁻⁵ cells/well) were isolated and then incubated with freshly isolated mitomycin C (25 μg/ml)-treated splenocytes (5×10⁵ cells/well) from WT mice in complete medium in the presence of 200 μg/mI OVA in 96-well plates (250 μl/well) for 96 hours.

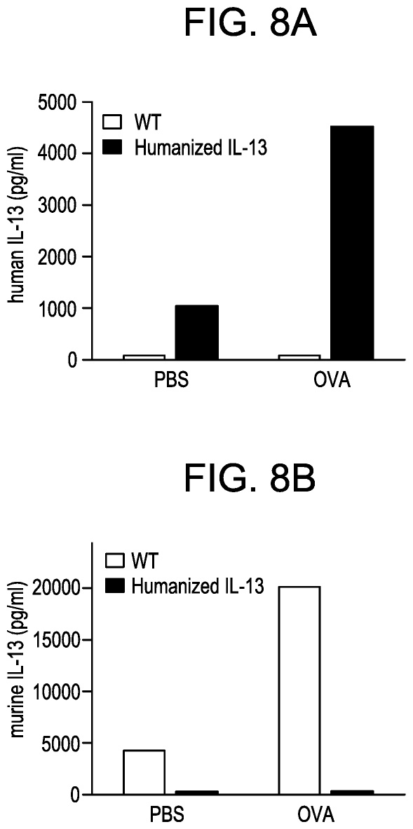

Cell-free culture supernatants were collected and tested for cytokine production. Human and murine IL-13 (DuoSet, R&D Systems, Minneapolis, Minn.) concentrations were determined by ELISA according to the manufacturer's protocol. As expected, CD4⁺ T cells derived from humanized IL-13 mice after in vitro OVA restimulation produced human IL-13 and no murine IL-13 (FIG. 8, panel A). In contrast, CD4⁺ T cells derived from WT mice produced murine IL-13 and no murine IL-13 (FIG. 8, panel B).

### Airway Hyper-Reactivity

The anti-IL-13 antibodies 731 and 623 were tested in OVA-induced asthma models using the humanized IL-13 mice described above. For the measurement of airway reactivity to the intravenous administration of acetylcholine, a 24 day protocol was used. Briefly, mice were immunized by an intraperitoneal injection of OVA (10 μg; crude grade IV; Sigma) in PBS (0.2 ml). PBS alone was used as a control. Fourteen days after immunization, mice were anesthetized with a mixture of ketamine and xylazine [45 and 8 mg per kilogram of body weight (mg/kg), respectively] and challenged intratracheally with 50 μl of a 1.5% solution of OVA or an equivalent volume of PBS as a control.

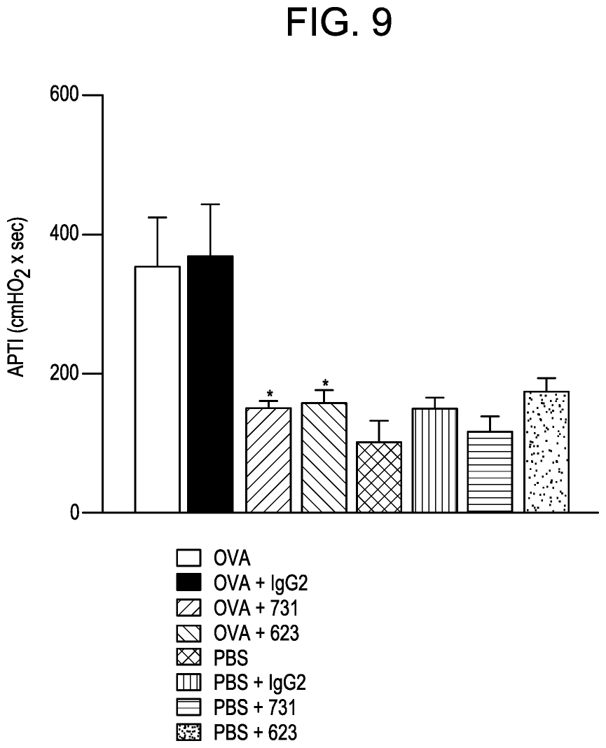

Seven days after the first antigen challenge, mice were challenged again intratracheally with either OVA or PBS. The 731 and 623 antibodies were administered intraperitoneally at a dose of 100 μg/mouse one day before each challenge (days 13 and 20). Control mice received PBS or an irrelevant IgG2 as isotype control. Three days after the final intratracheal challenge, mice were anesthetized with sodium pentobarbital (90 mg/kg), intubated, ventilated at a rate of 120 breaths/min with a constant tidal volume of air (0.2 ml), and paralyzed with decamethonium bromide (25 mg/kg). After a stable airway pressure was established, acetylcholine was injected intravenously (50 μg/kg), and the dynamic airway pressure was measured for 5 min. The airway hyperresponsiveness (AHR) to the acetylcholine challenge was measured. The airway hyperresponsiveness to acetylcholine challenge is defined by the time-integrated rise in peak airway pressure [airway-pressure-time index (APTI) in centimeters of H₂O×seconds]. * P<0.05, compared to the OVA+IgG2 control group [one-way analysis of variance (ANOVA) followed by Fisher's least significant difference test for multiple comparisons]. Treatment with 731 or 623 resulted in a complete reversal of OVA-induced AHR (FIG. 9). In this example, complete reversal means that the addition of the antibody with OVA results in an effect similar to one in which there is no OVA and only antibodies are added (e.g., IgG2).

### OVA-Induced Mucus Production

An 18 days protocol was used for the measurement of OVA-induced mucus production.

After subcutaneous priming with Ovalbumin (OVA, 25 μg; crude grade IV) (Sigma) in 2 mg Imject Alum on days 0 and 7, mice were anesthetized with isofluorane and challenged intranasally with 50 μl of a 1.5% solution of OVA in PBS on days 14, 15, and 17. Control mice received alum as priming or PBS as challenge.

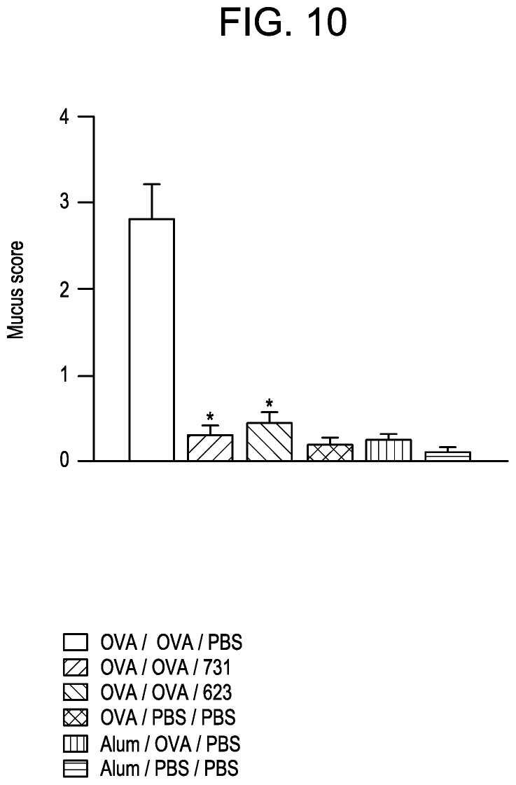

The 731 and 632 antibodies were administered intraperitoneally at a dose of 100 μg/mouse on days 13, 15, and 17. Control mice received PBS. On day 18 mice were sacrificed and lungs were collected after being perfused. Lung tissue, including central and peripheral airways, was fixed in 10% formalin, washed in 70% ethanol, dehydrated, embedded in glycol methacrylate, cut into 4-μM sections, mounted on slides, and stained with hematoxylin and eosin, plus Periodic acid-Schiff (PAS). Lung sections (one section per animal) were examined at 20× magnification. Five fields were selected randomly and for each section the number of bronchi was counted in each field. Sections were scored on a scale from 0 to 4 (0: <5% PAS⁺goblet cells; 1: 5 to 25%; 2: 25 to 50%; 3: 50 to 75%; 4: >75%). To obtain the histologic goblet cell score (expressed as arbitrary units; U) the sum of the airway scores from each lung was divided by the number of bronchi examined. Five out of eight mice died in the OVA treated group. No mice died in the other groups. Administration of 731 and 623 effectively reversed OVA-induced increase in mucus-containing cells in the airways (FIG. 10) Data are mean±SE. n=3 for OVA/OVA/PBS group (initially n=8); n=8 for OVA/OVA/731 group, n=4 for OVA/OVA/623 group; n=4 for OVA/PBS/PBS group, n=5 for Alum/OVA/PBS, and Alum/PBS/PBS groups. *p<0.01 vs OVA/OVA/PBS group by unpaired Student t-test.

### Example: Structural Analysis of Antibodies

The variable heavy chains and the variable light chains for the antibodies shown in Table 1 were sequenced to determine their DNA sequences. The complete sequence information for all anti-IL-13 antibodies are shown in the sequence listing submitted herewith, including nucleotide and amino acid sequences.

Table 18 shows the amino acid sequences of the heavy chain genes for a variety of the IL-13 antibodies described herein. Table 18 also shows the amino acid sequences corresponding to the CDRs and framework regions for each antibody, along with a comparison to its germline sequence.

Table 19 shows the amino acid sequences of the kappa light chain genes for a variety of the IL-13 antibodies described herein. Table 19 also shows the amino acid sequences corresponding to the CDRs and framework regions for each antibody, along with a comparison to its germline sequence.

Table 20 shows the amino acid sequences of the lambda light chain genes for a variety of the IL-13 antibodies described herein. Table 20 also shows the amino acid sequences corresponding to the CDRs and framework regions for each antibody, along with a comparison to its germline sequence.

TABLE 18

| | | | | |
|---|---|---|---|---|
| Single | | | | |
| Cell | V Heavy/D/J | FR1 | CDR1 | FR2 |

| | | | | |
|---|---|---|---|---|
| — | Germline | EVQLVESGGGLVKPGGSLRLSCAAS | GFTFSSYSMN | WVRQAPGKGLEWVS |
| | VH3-21/D1-26/JH3b | | | |
| 157 | | EVQLVESGGGLVKPGGSLRLSCAAS | GFTFSSYSMN | WVRQAPGKGLEWVS |
| 183 | | EVQLVESGGGLVKPGGSLRLSCAAS | GFTFSSYSMN | WVRQAPGKGLEWVS |
| 176 | | EVQLVESGGGLVKPGGSLRLSCAAS | GFTFSSYSMN | WVRQAPGKGLEWVS |
| 243 | | EVQLVESGGGLVKPGGSLRLSCAAS | GFTFSSYSMN | WVRQAPGKGLEWVS |
| 264 | | EVQLVESGGGLVKPGGSLRLSCAAS | GFTFSSYSMN | WVRQAPGKGLEWVS |
| | Germline | QVQLQESGPGLVKPSETLSLTCTVS | GGSISSYYWS | WIRQPPGKGLEWIG |
| 353 | VH4-59/D2-21/JH3B | QVQLQESGPGLVKPSETLSLTCTVS | GGSISTYYWS | WIRQPPGKGLEWIG |
| — | Germline | EVQLLESGGGLVQPGGSL?RLSCAAS | GFTFSSYAMS | WVRQAPGKGLEWVS |
| | VH3-23/D6-19/JH6B | | | |
| 713 | | EVQLLESGGGLVQPGGSL?RLSCAAS | GFTFSSYAMS | WVRQAPGKGLEWVS |
| 731 | | EVQLLESGGGLVQPGGSL?RLSCAAS | GFTFSSYAMS | WVRQAPGKGLEWVS |
| — | Germline | EVQLLESGGGLVQPGGSL?RLSCAAS | GFTFSSYAMS | WVRQAPGKGLEWVS |
| | VH3-23/D3-35H4B | | | |
| 785 | | EVQLLESGGGLVQPGGSL?RLSCAAS | GFTFSSYAMS | WVRQAPGKGLEWVS |
| — | Germline | QVQLQESGPGLVKPSETLSLTCTVS | GGSISSYYWS | WIRQPAGKGLEWIG |
| | VH4-4/D5-5/JH4B | | | |
| 693 | | QVQLQESGPGLVKPSETLSLTCSVS | GGSISSYYWS | WIRQPAGKGLEWIG |
| — | Germline | EVQLVESGGGLVKPGGSLRLSCAAS | GFTFSNAWMS | WVRQAPGKGLEWVG |
| | VH3-15/D1-26/JH6B | | | |
| 623 | | EVQLVESGGGLVKPGGSLRLSCAAS | GFTFSNAWMS | WVRQAPGKGLEWVG |
| 643 | | EVQLVESGGGLVKPGGSLRLSCAAS | GFTFSNAWMS | WVRQAPGKGLEWVG |
| 602 | | EVQLVESGGGLVKPGGSLRLSCAAS | GFTFSNAWMS | WVRQAPGKGLEWVG |
| — | Germline | QVQLVESGGGVVQPGRSLRLSCAAS | GFTFSSYGMH | WVRQAPGKGLEWVA |
| | VH3-33/D6-19/JH5B | | | |
| 11.18 | | QVQLVESGGGVVQPGRSLRLSCVAS | GFTFSSYDMH | WVRQAPGKGLEWVA |
| — | Germline | EVQLVESGGGLVKPGGSLRLSCAAS | GFTFSSYSMN | WVRQAPGKGLEWVS |
| | VH3-21/NA/JH6B | | | |
| 356 | | EVQLVESGGGLVKPGGSLRLSCAAS | GFTFSDYNMH | WVRQAPGKGLEWVS |

| Single | | | | |
|---|---|---|---|---|
| Cell | CDR2 | FR3 | CDR3 | FR4 |
| — | SISSSSSYIYYADSVKG | RFTISRDNAKNSLYLQMNSLRAEDTAVYYCAR | | WGQGTMVTVSS |
| 157 | YISTSYNYIYYADSVKG | RFTISRDNAKNSLYLQMNSLRAEDTAVYYCAR | DQVGATLDAFDI | WGQGTMVTVSS |
| 183 | YISSSYNYIYYGDSVKG | RFTISRDNAKNSLYLQMNSLRAEDTAVYYCAR | DQVGATLDAFDI | WGQGTMVTVSS |
| 176 | YISTSNSYIYYADSVKG | RFTISRDNAKNSLYLQMNSLRAEDTAVYYCAR | DQVGATLDAFDI | WGQGTMVTVSS |
| 243 | YISTSNSYIYYADSVKG | RFTISRDNAKNSLYLQMNSLRAEDTAVYYCAR | DQVGATLDAFDI | WGQGTMVTVSS |
| 264 | YISTSNSYIYYADSVKG | RFTISRDNAKNSLYLQMNSLRAEDTAVYYCAR | DQVGATLDAFDI | WGQGTMVTVSS |
| | YIYYSGSTNYNPSLKS | RVTISVDTSKNQFSLKLSSVTAADTAVYYCAR | | WGQGTMVTVSS |
| 353 | YIYYSGSTNYNPSLKS | RVTISVDTSKNQFSLKLSSVTAADTAVYYCAR | DGGHYWDDAFDI | WGQGTMVTVSS |
| — | AISGSGGSTYYADSVKG | RFTISRDNSKNTLYLQMNSLRAEDTAVYYCAK | | WGQGTTVTVSS |
| 713 | AFSGSGGSTYYADSVKG | RFTISRDNSKNTLYLQMNSLRAEDTAVYYCVQ | DGLGPYFYNYGMDV | WGQGTTVTVSS |
| 731 | AFSGSGGSTYYADSVKG | RFTISRDNSKNTLYLQMNSLRAEDTAVYYCVQ | DGLGPYFYNYGMDV | WGQGTTVTVSS |
| — | AISGSGGSTYYADSVKG | RFTISRDNSKNTLYLQMNSLRAEDTAVYYCAK | | WGQGTLVTVSS |
| 785 | AISGSGGSTYYADSVKG | RFTISRDNSKNTLYLQMNSLRAEDTAVYYCAK | ADFWSGTLWGFDY | WGQGTLVTVSS |
| — | RIYTSGSTNYNPSLKS | RVTMSVDTSKNQFSLKLSSVTAADTAVYYCAR | | WGQGTLVTVSS |
| 693 | RIYMTGRTNYNSSLKS | RVTMSIDTSKNQLSLKLSFMTAADTAVYYCAR | ESGSSYSYDY | WGQGTLVTVSS |
| — | RIKSKTDGGTTDYAAPVKG | RFTISRDDSKNTLYLQMNSLKTEDTAVYYCTT | | WGQGTLVTVSS |
| 623 | RIRSEIDGGTTNYAAPVKG | RFTISRDDSKNTLYLQMNSLKTEDTAVYYCAT | DQVGAYYGDYYGMDV | WGQGTLVTVSS |
| 643 | RIRSEIDGGTTNYAAPVKG | RFTISRDDSKNTLYLQMNSLRTEDTAVYYCAT | DQVGAYYGDYYGMDV | WGQGTLVTVSS |
| 602 | RIRSKIDGGTINYAAPVKG | RFTISRDDSKNTLYLQMNSLKTEDTAVYYCAT | DQVGAYYGDYYGMDV | WGQGTLVTVSS |
| — | VIWYDGSNKYYADSVKG | RFTISRDNSKNTLYLQMNSLRAEDTAVYYCAR | | WGQGTLVTVSS |
| 11.18 | VIWYDGSNKYYADSVQG | RFTISRDNSKNTLYLQMNSLRAEDTAVYYCTS | EDSSGWYDGWFDP | WGQGTLVTVSS |
| — | SISSSSSYIYYADSVKG | RFTISRDNAKNSLYLQMNSLRAEDTAVYYCAR | | WGQGTTVTVSS |
| 356 | SISYSSTYIYYADSVRG | RFTISRDNAKNSLYLQMNSLRAEDTAVFYCAR | EDYYYYGLDV | WGQGTTVTVSS |

TABLE 19

| Single | Light--V | | | |
|---|---|---|---|---|
| Cell | Kappa/J | FR1 | CDR1 | FR2 |
| | Germline | DIQMTQSPSSLSASVGDRVTITC | RASQGIRNDLG | WYQQKPGKAPKRLIY |
| | A30(Vk1)/JK3 | | | |
| 157 | | DIQMTQSPSSLSASVGDRVTITC | RASQGIGDDLG | WYQQKPGKAPKRLIY |
| 183 | | DIQMTQSPSSLSASVGDRVTITC | RASQGIGDDLG | WYQQKPGKAPKRLIY |
| 176 | | DIQMTQSPSSLSASVGDRVTFTC | RASQDITDDLG | WYQQKPGKAPKRLIY |
| 243 | | DIQMTQSPSSLSASVGDRVTFTC | RASQDITDDLG | WYQQKPGKAPKRLIY |
| 264 | | DIQMTQSPSSLSASVGDRVTFTC | RASQDITDDLG | WYQQKPGKAPKRLIY |
| 353 | | DIQMTQSPSSLSASVGDRVTITC | RASQGIRNDLD | WYQQKPGKAPKRLIY |
| — | Germline | DIQMTQSPSSLSASVGDRVTITC | RASQGISNYLA | WYQQKPGKVPKLLIY |
| | A20/JK3 | | | |
| 11.18 | | DIQMTQSPSSLSASVGDRVTITC | RASQGISNYLA | WYQQKPGKVPKVLIY |
| — | Germline | DIQMTQSPSSLSASVGDRVTITC | RASQGIRNDLG | WYQQKPGKAPKRLIY |
| | A20/JK2 | | | |
| 356 | | DIQMTQSPSSLSASVGDRVTITC | RASQGIRNDLG | WYQQKPGKAPKRLIY |

| Single | | | | |
|---|---|---|---|---|
| Cell | CDR2 | FR3 | CDR3 | J |
| | AASSLQS | GVPSRFSGSGSGTEFTLTISSLQPEDFATYYC | | FGPGTKVDIK |
| 157 | AASSLQS | GVPSRFSGSGSGTEFTLTISSLQPEDFATYYC | LQHNSYPFT | FGPGTRVDIK |
| 183 | AASSLQS | GVPSRFSGSGSGTEFTLTISSLQPEDFATYYC | LQHNSYPFT | FGPGTKVDIK |
| 176 | AASSLQS | GVPPRFSGSGSGTEFTLTISSLQPEDFATYYC | LQHNSYPFT | FGPGTKVDIR |
| 243 | AASSLQS | GVPSRFSGSGSGTEFTLTISSLQPEDFATYYC | LQHNSYPFT | FGPGTKVDIR |
| 264 | AASSLQS | GVPSRFSGSGSGTEFTLTISSLQPEDFATYYC | LQHNSYPFT | FGPGTKVDIR |
| 353 | DASSLQS | GVPSRFSGSGSGTEFTLTISSLQPEDFATYYC | LQHDSYPFT | FGPGTKVDIK |
| — | AASTLQS | GVPSRFSGSGSGTDFTLTISSLQPEDVATYYC | | FGPGTKVDTK |
| 11.18 | AASTLQS | GVPSRFSGSGSGTDFTLTISSLQPEDVATYYC | QKYNSAPFT | FGPGTKVDIK |
| — | AASSLQS | GVPSRFSGSGSGTEFTLTISSLQPEDFATYYC | | FGQGTKLEIK |
| 356 | AASSLQS | GVPSRFSGSGSGTEFTLTISSLQPEDFATYYC | LQHNSYPWT | FGQGTKVEIK |

TABLE 20

| Single | Light-V | | | |
|---|---|---|---|---|
| Cell | Lamda/J | FR1 | CDR1 | FR2 |
| — | Germline | SYELTQPPSVSVSPGQTASIT?C | SGDKLGDKYAC | WYQQKPGQSPVLVIY |
| | V2-1/JL1 | | | |
| 713 | | SYELTQPPSVSVSPGQTASIT?C | SGDKLGDKYTC | WFQQKPGQSPVLVIY |
| 731 | | SYELTQPPSVSVSPGQTASIT?C | SGDKLGDKYAC | WFQQKPGQSPVLVIY |
| — | Germline | SYVLTQPPSVSVAPGQTARITC | GGNNIGSKSVH | WYQQKPGQAPVLVVY |
| | V2-14/JL2 | | | |
| 693 | | SYVLTQPPSVSVAPGQTARITC | GGNNIGSKGVH | WYQQKPGQAPVLVVY |
| 785 | | SYVLTQPPSVSVAPGQTARITC | GGNNIGNKIVH | WYQQKPGQAPVLVVY |
| — | Germline | SYELTQPPSVSVSPGQTARITC | SGDALPKKYAY | WYQQKSGQAPVLVIY |
| | V2-7/JL3 | | | |
| 623 | | SYELTQPPSVSVSPGQTARITC | SGDALPEKYAY | WYQQKSGQAPVLVIY |
| 643 | | SYELTQPPSVSVSPGQTARITC | SGDALPEKYAY | WYQQKSGQAPVLVIY |
| 602 | | SYELTQPPSVSVSPGQTARITC | SGDALPEKYAY | WYQQKSGQAPVLVIY |

| Single | | | | |
|---|---|---|---|---|
| Cell | CDR2 | FR3 | CDR3 | J |
| — | QDSKRPS | GIPERFSGSNSGNTATLTISGTQAMDEADYYC | | FGTGTKVTVL |
| 713 | HDSKRPS | GIPERFSGSNSGDTATLTISGTQAMDEADYYC | QAWDSSTYV | FGTGTKVTVL |
| 731 | HDSKRPS | GIPERFSGSNSGDTATLTISGTQAMDEADYYC | QAWDSSTYV | FGTGTKVTVL |
| — | DDSDRPS | GIPERFSGSNSGNTATLTISRVEAGDEADYYC | | FGGGTKLTVL |
| 693 | DDSDRPS | GIPERFSGSNSGNTATLTISRVEAGDEADYYC | QVWVSSSDHHVV | FGGGTKLTVV |
| 785 | DDSDRPS | GIPERFSGSNSGNTATLTISRVEAGDEADYYC | QVWDSSSDHVV | FGGGTKLTVL |
| — | EDSKRPS | GIPERFSGSSSGTMATLTISGAQVEDEADYYC | | FGGGTKLTVL |
| 623 | EDSKRPS | GIPERFSGSSSGTMATLTISGAQVEDEADYYC | HSTDSSGNHGV | FGGGTKLTVL |
| 643 | EDSKRPS | GIPERFSGSSSGTMATLTISGAQVEDEADYYC | HSTDSSGNHGV | FGGGTKLTVL |
| 602 | EDTKRPS | GIPERFSGSSSGTMATLTISGAQVEDEADYYC | YSTDSSGNHGV | FGGGTKLTVL |

### Example 5: Generation of Affinity Matured Anti-IL13 Antibodies

Based on the sequences from the Xenomouse® derived antibodies, a novel use of mammalian recombination signal sequence (RSS)-directed recombination for Complementarity-Determining Regions (CDR)-targeted protein engineering to close the species affinity gap of antibody 731 (Ab731).

Using this non-hypothesis driven affinity maturation method, we generated multiple antibody variants with improved IL-13 affinity, including the highest affinity antibody reported to date to human IL-13 with high cross-reactivity to cyno IL-13.

HuTARG technology is a novel RSS-recombination-based protein engineering platform coupled to cell surface display in a mammalian cell culture system. Briefly, DNA encoding complementarity determining regions (CDRs) in the heavy and light chain of Ab731 were engineered by standard molecular biology methods to contain RSS sites. The resulting plasmid pool encoded individual CDRs where a RSS integration was targeted; successful integration of RSS signals into each CDR was confirmed by terminal restriction fragment length polymorphism (T-RFLP). The resulting constructs were stably integrated as a pool into the HuTARG cell line using the Cre-Lox system. HuTARG cells are recombination-competent mammalian cells, where the RAG-1-mediated recombinase activity is induced under tetracycline treatment. The induction of recombination results in each cell undergoing a unique rearrangement, that involves the removal of the RSS-cassette and, in the presence of terminal deoxynucleotidyltransferase (TdT), a double strand break repair, resulting in imperfect joining of recombined segments and the creation of sequence variation in human IgG antibody. This antibody is subsequently expressed on the surface of the cells because of defined genoic integration into the lox P site, each cell expresses a unique, and single specificity, antibody. As heavy and light chain sequences were targeted separately, two individual pools of vectors were generated and used to create diversified heavy and light chain antibody cell surface display libraries. Since recombination occurs in the cell directly and there is no need for a transformation step, the limitation of library complexity is determined by cell number. In this case, we utilized complexity of 342E6 cells for the diversified heavy chain and 320E6 cells for diversified light chain. Improved affinity variants (as determined by higher than parental antibody binding) were isolated by three rounds of FACS sorting by surface staining with 36 pM of recombinant soluble cyno IL-13. Variant antibody sequences selected to have improved affinity were found to include insertions and substitutions that were relatively evenly distributed across the enriched light chains consistent with the frequency of RSS-integration. However, CDR-H3 was the least altered, which is perhaps not surprising as CDR-H3 is the principal determinant of epitope recognition for B cells leaving the bone marrow. Insertions and deletions were the dominant types of mutations observed in the affinity enriched FACS sorted cells.

PCR-rescued antibody sequences were cloned and transiently expressed on the surface of HEK293T cells to rank their binding to soluble human and cyno IL-13 by FACS analysis. Geometric mean values of fluorescence for binding to the target protein were compared in three gates based on cell surface IgG expression. Of the heavy chain variants that showed improved cyno IL-13 binding two were confirmed by KinExA to have an affinity higher than their parent antibody: heavy chain 1 (HC1) and HC2 (FIG. 1C, Supplementary Table 20(a). A similar analysis was undertaken for light chain variants and resulted in the identification of three light chain sequences (LC1, LC2, and LC3) with higher than parental antibody binding. These light chain variants were subsequently combined by checkerboard pairing with the best heavy chain variants in all possible permutations. This resulted in the identification of three antibodies, MMAb3, MMAb5, and MMAb7, that had improved affinity to both cyno and human IL-13, and showed stronger binding than antibodies with individual mutations in the light chain or heavy chain. Their biological potency was assessed for neutralization of Eotaxin 1-release from normal human dermal fibroblasts (NHDF) cells stimulated with human or cyno IL-13. All antibodies were found to be potent inhibitors against both ligands, with EC50s that were limited by the concentration of IL-13 in the assay. In contrast, the parental Ab731 showed no activity against cyno IL13.

Formal affinity of newly generated antibody variants to the human and cyno IL13 targets was determined using a KinExA-based affinity determination demonstrated that MMAb3 had affinities of 5.1 μM to cyno IL-13 and 34 fM to human IL-13, thus showing a 700-fold improvement and a 56-fold improvement, respectively. To our knowledge the affinity of MMAb7 (as well as MMAb5 at 142 fM), to the human IL-13 protein is one of the highest described in the literature. We previously reported an in vivo generated anti-IL8 antibody with sub-picomolar (610 fM) affinity, measured by KinExA technology. Other examples of very high affinity antibodies include affinity maturation by site-directed mutagenesis of a murine anti-IL1b antibody, XMA005 and subsequent humanization of that, XOMA 052, yielding sub-picomolar antibodies (240 fM and 300 fM, respectively), measured similarly by KinExA technology (Owyang, A. M. et al. XOMA 052, a potent, high-affinity monoclonal antibody for the treatment of IL-Ibeta-mediated diseases. mAbs 3, 49-60 (2011)). An engineered anti-fluorescein single chain antibody was also reported to have a Kd of 270 fM to the small molecule hapten fluorescein, measured similarly by an equilibrium binding method (Boder, E. T., Midelfort, K. S. & Wittrup, K. D. Directed evolution of antibody fragments with monovalent femtomolar antigen-binding affinity. Proceedings of the National Academy of Sciences of the United States of America 97, 10701-10705 (2000); Midelfort, K. S. et al. Substantial energetic improvement with minimal structural perturbation in a high affinity mutant antibody. Journal of molecular biology 343, 685-701(2004)).

TABLE 20(a)

| | | | | | | | Cy IL-13 | | Hu IL-13 | |
|---|---|---|---|---|---|---|---|---|---|---|
| Anti- | LC | | | HC | | | Kd (pM) | IC₅₀ (pM) | Kd (pM) | IC₅₀ (pM) |
| body | CDR-L1 | CDR-L2 | CDR-L3 | CDR-H1 | CDR-H2 | CDR-H3 | (95% CI) | (95% CI) | (95% CI) | (95% CI) |
| Ab731 | SGDKLG | HDSKRPS | QAWDSS | SYAMS | AFSGSGGST | DGLGPYF | 3550 | >500 | 1.9 | 45.4 |
| | DKYTC | | TYV | | YYADSVKG | YNYGMDV | (2310-5340) | (NA) | (1.0-3.1) | (37.9-54.5) |
| MMAb3 | ------ | ------- | ------ | ---G- | --------- | ------- | 18.7 | 93.6 | 0.295 | 34.7 |
| (LC1/ | ---SF | | --- | | -------- | ------- | (11-32) | (72.1-121.5) | (0.09-0.69) | (27.8-43.3) |
| HC2) | | | | | | | | | | |
| MMAb5 | ------ | ------- | ------ | ---G- | --------- | ------- | 12.4 | 102.1 | 0.142 | 49.0 |
| (LC2/ | ---AF | | --- | | -------- | ------- | (8-19) | (81.9-127.3) | (0.03-0.35) | (40.9-58.8) |
| HC2) | | | | | | | | | | |
| MMAb7 | ------ | ------- | ------ | ----- | ----WDV-- | ------- | 5.1 | 90.8 | 0.034 | 41.3 |
| (LC1/ | ---SF | | --- | | -------- | ------- | (3.1-7.9) | (73.9-111.7) | (0.002-0.105) | (35.1-48.7 |
| HC1) | | | | | | | | | | |

### Example 6: Epitopes and Co-Crystalization

Co-crystal structures of the novel antibodies with cynomologus and human IL-13. To understand the molecular determinants of the very high affinity interaction of the MMAb3 antibody with cyno and human IL-13, The crystal structure of cyno IL-13 in complex with MMAb3 fragment antigen-binding (Fab) at 2.1 A resolution (FIG. 2A) and subsequently created homology models for MMAb1 and MMAb2 bound to the same ligand. The crystal structure of cyno IL-13 in complex with MMAb3 Fab revealed that the Helix-C of IL-13 is oriented parallel to the Fab cleft and is interposed between the Fab heavy and light chains. The total buried solvent accessible surface area (SASA) of 1784.8 Å² is greater than that observed for average antibody-antigen interfaces (1500-1600 Å²).²⁵ The overall shape complementarity score (Sc) of 0.714 suggests an even higher degree of complementarity for the IL-13-Fab interface than average (0.64-0.68)²⁶, indicating an extensive and fitted interface for the two molecules.

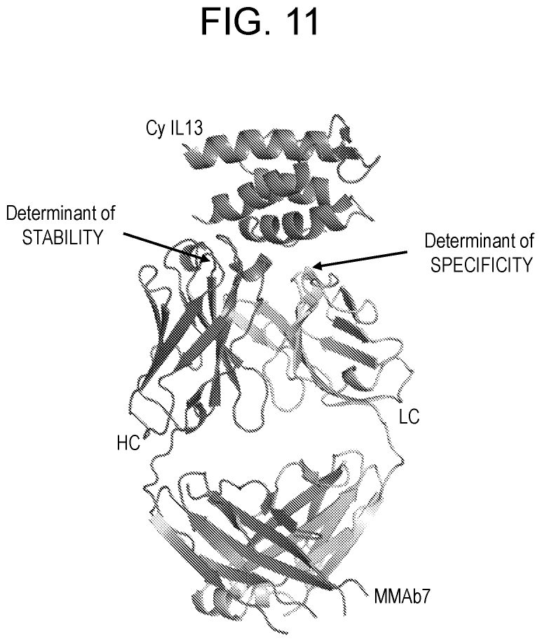

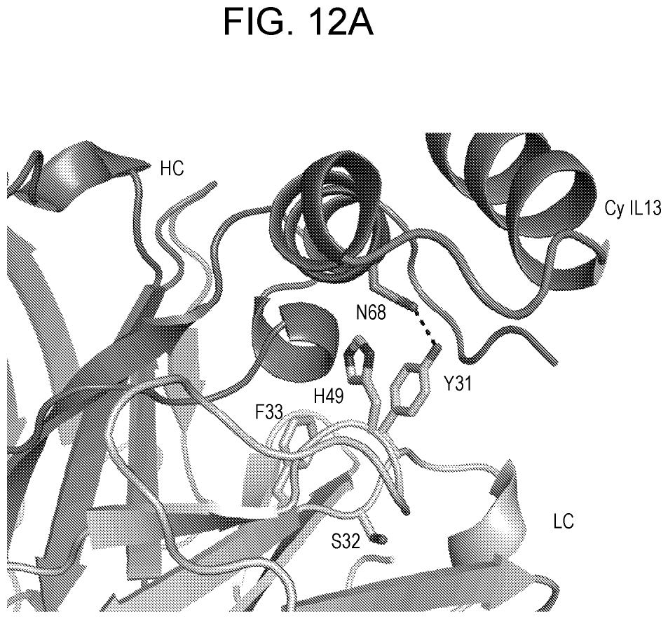

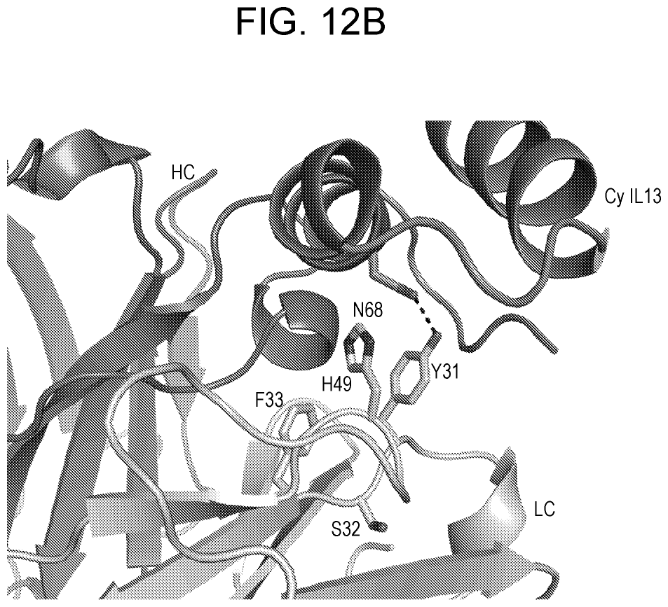

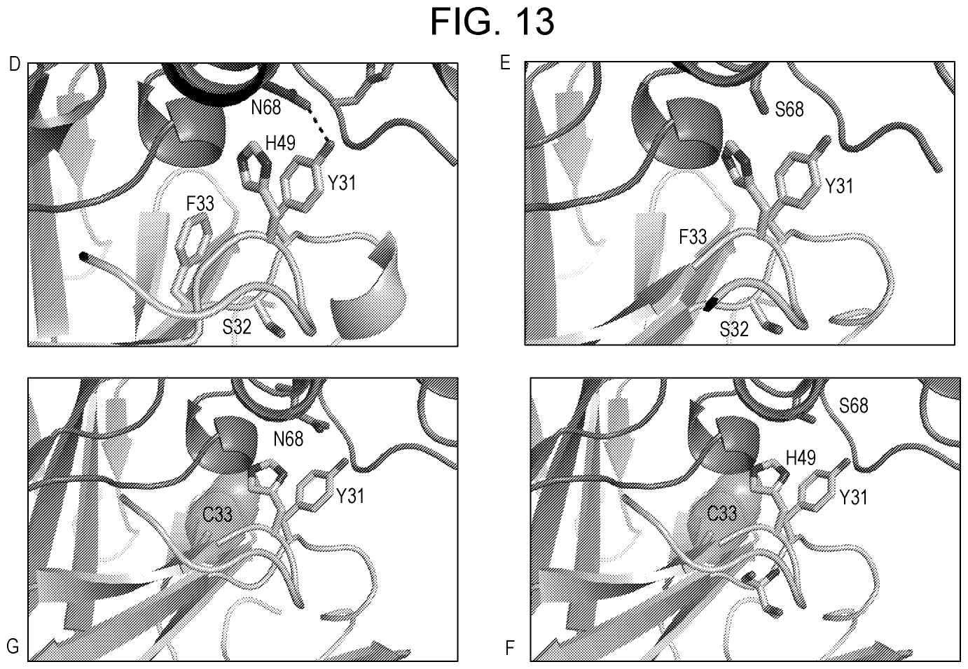

The crystal structure provided an explanation of the high affinity of MMAb3 towards cyno and human IL-13. MMAb3 deviates from the parental Ab731 via three consecutive residues in the CDR-H2 (Trp 54/Asp 55/Val 56 versus Ser 54/Gly 55/Gly 56) and two successive residues on the CDR-L1 (Ser 32/Phe 33 versus Thr 32/Cys 33) (FIG. 11). The first set of residues induces the formation of a π-π stacking channel wherein the engineered residue Trp 54 from CDR-H2 picks up π-π stacking interactions with Pro 103 of CDR-H2, leading to similar contacts further along the channel with CDR-H2 Tyr 104 and IL-13 residues Pro 72 and His 73 engaging in mostly van der Waals contacts (FIG. 13). It is also likely that the presence of Asp 55 and Val 56 in place of parental Gly 55 and Gly 56 serve to further stabilize the backbone of the CDR-H2 loop, although this was difficult to assess energetically due to the conformational variability associated with the presence of two subsequent Gly residues. The central role of Trp 54 to the binding interface is also evidenced by its buried surface area of 170.1 Å², which makes up nearly 10% of the total SASA. Structural evidence therefore suggests a stabilizing role for Trp 54, rather than being a direct determinant of affinity or specificity; two properties we believe are more likely driven by interactions from the CDR-L1 (FIGS. 12A and B). As the MMAb3 and Ab731 CDR-L1 paratopes differ with regards to two residues (Ser 32/Phe 33 versus Thr 32/Cys 33), we attempted to identify in this region the change in binding interactions that conferred greater cross-reactivity between cyno and human IL-13. In the crystal structure, Asn 68 from cyno IL13 is positioned between Tyr 31 and the IL13 backbone carboxyl from residues 73-76. The structure suggests that the tight space created by these contacts locks Asn 68 in a conformation that allows binding, albeit through suboptimal hydrogen bonding with Tyr 31 (FIGS. 12A and B). In human IL-13, a Ser residue replaces Asn 68 (FIG. 13). Ser 68, given its smaller size and greater distance from the surrounding IL-13 backbone residues, is conformationally less restricted and better positioned to establish a stronger hydrogen bond with the hydroxyl group of Tyr 31, resulting in tighter binding (consistent with experimental data). In either case, and very difficult to predict a priori, the conformation of Tyr 31 is likely stabilized by the downstream engineered residue Phe 33, with the bulky aromatic ring occupying a cavity and preventing rotamer flipping, as opposed to the parental antibody, which has a Cys 33 (FIG. 13) and a surrounding cavity. In the context of the parental antibody in complex with human IL-13, the higher affinity of the Ser 68:Tyr 31 interaction favors an outward conformation of Tyr 31 despite the presence of a cavity surrounding Cys 33. This contrasts with cyno IL-13, where the lower affinity of the Asn 68: Tyr31 interaction allows the tyrosine to swing back and occupy the cavity surrounding Cys 33, thus reaching an internal energy minimum. However, this significantly lowers affinity of the parental antibody towards cyno IL-13.

### Example 7: High Affinity Anti-IL-13 Antibodies

A number of variants were made to the Mmab7 light and heavy chains for stability and viscosity. Those high affinity anti-IL13 amino acid sequences are listed below in Tables 21-22.

TABLE 21
High Affinity anti-IL-13 antibodies

| | | Seq ID |
|---|---|---|
| Description | Amino Acid Sequence | NO: |
| Light Chain | MAWALLLLTLLTQGTGSWASYELTQPPSVSVSPGQTASIT | 22 |
| Variant 4534 | CSGDKLGDKYAFWFQQKPGQSPVLVIYHDAKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 23 |
| IgG1 variant | RLSCAASGFTFSSYAGSWVRQAPGKGLEWVSAFSGSGGST | |
| 122160 | YYADSVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 24 |
| variant 124535 | CSGDKLGDKYAFWFQQKPGQSPVLVIYHDSKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 25 |
| IgG1 variant | RLSCAASGFITSSYAGSWVRQAPGKGLEWVSAFSGSGGST | |
| 124535 | YYADSVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 26 |
| variant 124536 | CSGDKLGDKYAFWFQQKPGQSPVLVIYHDAKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 27 |
| IgG1 variant | RLSCAASGFTFSSYAGSWVRQAPGKGLEWVSAFSGSGGST | |
| 124535122160 | YYADSVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 28 |
| variant 122159 | CSGDKLGDKYAFWFQQKPGQSPVLVIYHDSKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 29 |
| IgG1 variant | RLSCAASGFTFSSYAGSWVRQAPGKGLEWVSAFSGSGGST | |
| 124535SEQ12453 | YYADAVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 30 |
| variant 122159 | CSGDKLGDKYAFWFQQKPGQSPVLVIYHDSKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 31 |
| IgG1 variant | RLSCAASGFTFSSYAGSWVRQAPGKGLEWVSAFSGSGGST | |
| 124535124538 | YYADSVKGRFTISRDNSKNTLYLQMNSLRAEDTAVYYCVQ | |
| | DALGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Heavy Chain | MDMRVPAQLLGLLLLWLRGARCEVQLLESGGGLVQPGGSL | 32 |
| IgG1 variant | RLSCAASGFTFSSYAGSWVRQAPGKGLEWVSAFSGSGGST | |
| 124535SEQ124538 | YYADSVKGRFTISRDNSKNTLYLQMNSLRAEDTAVYYCVQ | |
| | DALGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 33 |
| variant 122159 | CSGDKLGDKYAFWFQQKPGQSPVLVIYHDSKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 34 |
| IgG1 variant | RLSCAASGFTFSSYAGSWVRQAPGKGLEWVSAFSGSGGST | |
| 124535124539 | YYADSVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 35 |
| variant 124534 | CSGDKLGDKYAFWFQQKPGQSPVLVIYHDAKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 36 |
| IgG1 variant | RLSCAASGFTFSSYAGSWVRQAPGKGLEWVSAFSGSGGST | |
| 124535124537 | YYADAVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 37 |
| variant 124535 | CSGDKLGDKYAFWFQQKPGQSPVLVIYHDSKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 38 |
| IgG1 variant | RLSCAASGFTFSSYAGSWVRQAPGKGLEWVSAFSGSGGST | |
| 124535124537 | YYADAVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 39 |
| variant 124536 | CSGDKLGDKYAFWFQQKPGQSPVLVIYHDAKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 40 |
| IgG1 variant | RLSCAASGFTFSSYAGSWVRQAPGKGLEWVSAFSGSGGST | |
| 124535124537 | YYADAVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 41 |
| variant 124534 | CSGDKLGDKYAFWFQQKPGQSPVLVIYHDAKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 42 |
| IgG1 variant | RLSCAASGFTFSSYAGSWVRQAPGKGLEWVSAFSGSGGST | |
| 124535124539 | YYADSVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 43 |
| variant 124536 | CSGDKLGDKYAFWFQQKPGQSPVLVIYHDAKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 44 |
| IgG1 variant | RLSCAASGFTFSSYAGSWVRQAPGKGLEWVSAFSGSGGST | |
| 124535124539 | YYADSVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 45 |
| variant 124534 | CSGDKLGDKYAFWFQQKPGQSPVLVIYHDAKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 46 |
| IgG1 variant | RLSCAASGFTFSSYAGSWVRQAPGKGLEWVSAFSGSGGST | |
| 124535124540 | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 47 |
| variant 124535 | CSGDKLGDKYAFWFQQKPGQSPVLVIYHDSKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 48 |
| IgG1 variant | RLSCAASGFTFSSYAGSWVRQAPGKGLEWVSAFSGSGGST | |
| 124535124540 | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 49 |
| variant 124536 | CSGDKLGDKYAFWFQQKPGQSPVLVIYHDAKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 50 |
| IgG1 variant | RLSCAASGFTFSSYAGSWVRQAPGKGLEWVSAFSGSGGST | |
| 124535124540 | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| Light Chain | MAWALLLLTLLTQGTGSWASYELTQPPSVSVSPGQTASIT | 51 |
| variant 124535 | CSGDKLGDKYAFWFQQKPGQSPVLVIYHDSKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| Heavy Chain | MDMRVPAQLLGLLLLWLRGARCEVQLLESGGGLVQPGGSL | 52 |
| IgG1 variant | RLSCAASGFTFSSYAGSWVRQAPGKGLEWVSAFSGSGGST | |
| 124535124539 | YYADSVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 81-704 H chain | MDMRVPAQLLGLLLLWLRGARCEVQLLESGGGLVQPGGSL | 53 |
| | RLSCAASGFTFSSYAGSWVRQAPGKGLEWVSAFSGSGGST | |
| | YYADSVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 81-704 Lchain | MAWALLLLTLLTQGTGSWASYELTQPPSVSVSPGQTASIT | 54 |
| | CSGDKLGDKYAFWFQQKPGQSPVLVIYHDSKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 27461-1 H chain | MDMRVPAQLLGLLLLWLRGARCEVQLLESGGGLVQPGGSL | 55 |
| | RLSCAASGFTFSSYAGSWVRQAPGKGLEWVSAFSGSGGST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 27461-1 L Chain | MAWALLLLTLLTQGTGSWASYELTQPPSVSVSPGQTASIT | 56 |
| | CSGDKLGDKYAFWFQQKPGQSPVLVIYHDAKRPSGIPERF | |
| | SGSNSGDTATLTISGTQAMDEADYYCQAWDSSTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 27463-2 H chain | MDMRVPAQLLGLLLLWLRGARCEVQLLESGGGLVQPGGSL | 57 |
| | RLSCAASGFTFSSYAGSWVRQAPGKGLEWVSAFSGSGGST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 27463-2 L Chain | MAWALLLLTLLTQGTGSWASYELTQPPSVSVSPGQTASIT | 58 |
| | CSGDKLGDKYAFWFQQKPGQSPVLVIYHDAKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAVIVAWKADSSPVKAGVETTTPSKQSNNKYAASSYLSLT | |
| | PEQWKSHRSYSCQVTHEGSTVEKTVAPTECS | |
| 29351-1 H Chain | MDMRVPAQLLGLLLLWLRGARCEVQLLESGGGLVQPGGSL | 59 |
| | RLSCAASGFTFSSYAMSWVRQAPGKGLEWVSAFSGWDVST | |
| | YYADSVKGRFTISRDNSKNTLYLQMNSLRAEDTAVYYCVQ | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 29351-1 L chain | MDMRVPAQLLGLLLLWLRGARCSYELTQPPSVSVSPGQTA | 60 |
| | SITCSGDKLGDKYSFWFQQKPGQSPVLVIYHDSKRPSGIP | |
| | ERFSGSNSGDTATLTISGTRAMDEADYYCQAWDSSTYVFG | |
| | TGTKVTVLGQPKAAPSVTLFPPSSEELQANKATLVCLISD | |
| | FYPGAVTVAWKADSSPVKAGVETTTPSKQSNNKYAASSYL | |
| | SLTPEQWKSHRSYSCQVTHEGSTVEKTVAPTECS | |
| 28886-2 H chain | MDMRVPAQLLGLLLLWLRGARCEVQLLESGGGLVQPGGSL | 61 |
| | RLSCAASGFTFSSYAMSWVRQAPGKGLEWVSAFSGWDVST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 28886-2 L chain | MAWALLLLTLLTQGTGSWASYELTQPPSVSVSPGQTASIT | 62 |
| | CSGDKLGDKYSFWFQQKPGQSPVLVIYHDAKRPSGIPERF | |
| | SGSNSGDTATLTISGTQAMDEADYYCQAWDASTYVFGTGT | |
| | KVTVLGQPKAAPSVTLFPPSSEELQANKATLVCLISDFYP | |
| | GAV1VAWKADSSPVKAGVETTTPSKQSNNKYAASSYLSLT | |
| | PEQWKSHRSYSCQVTHEGSTVEKTVAPTECS | |
| 29354-1 H chain | MDMRVPAQLLGLLLLWLRGARCEVQLLESGGGLVQPGGSL | 63 |
| | RLSCAASGFTFSSYAMSWVRQAPGKGLEWVSAFSGWDVST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 29354-1 H chain | MDMRVPAQLLGLLLLWLRGARCEVQLLESGGGLVQPGGSL | 64 |
| | RLSCAASGFTFSSYAMSWVRQAPGKGLEWVSAFSGWDVST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 29354-1 L chain | MAWALLLLTLLTQGTGSWASYELTQPPSVSVSPGQTASIT | 65 |
| | CSGKKLGKKYSFWFQQKPGQSPVLVIYHDAKRPSGIPERF | |
| | SGSNSGNTATLTISGTQAMDEADYYCQAWDASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 29355-1 L chain | MAWALLLLTLLTQGTGSWASYELTQPPSVSVSPGQTASIT | 66 |
| | CSGKKLGKKYSFWFQQKPGQSPVLVIYHDAKRPSGIPERF | |
| | SGSNSGDTATLTISGTQAMDEADYYCQAWKASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 29355-1 Light | MAWALLLLTLLTQGTGSWASYELTQPPSVSVSPGQTASIT | 67 |
| chain | CSGKKLGKKYSFWFQQKPGQSPVLVIYHDAKRPSGIPERF | |
| | MAWALLLLTLLTQGTGSWASYELTQPPSVSVSPGQTASIT | |
| | CSGKKLGKKYSFWFQQKPGQSPVLVIYHDAKRPSGIPERF | |
| | SGSNSGDTATLTISGTQAMDEADYYCQAWKASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 29357-1 H chain | MDMRVPAQLLGLLLLWLRGARCEVQLLESGGGLVQPGGSL | 68 |
| | RLSCAASGFTFSSYAMSWVRQAPGKGLEWVSAFSGWDVST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 293571 L Chain | MAWALLLLTLLTQGTGSWASYELTQPPSVSVSPGQTASIT | 69 |
| | CSGDKLGKKYSFWFQQKPGQSPVLVIYHDAKRPSGIPERF | |
| | SGSNSGNTATLTISGTQAMDEADYYCQAWKASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 29366-1 Heavy | MDMRVPAQLLGLLLLWLRGARCEVQLLESGGGLVQPGGSL | 70 |
| chain | RLSCAASGFTFSSYAMSWVRQAPGKGLEWVSAFSGWDVST | |
| | MDMRVPAQLLGLLLLWLRGARCEVQLLESGGGLVQPGGSL | |
| | RLSCAASGFTFSSYAMSWVRQAPGKGLEWVSAFSGWDVST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 29366-1 L chain | MAWALLLLTLLTQGTGSWASYVLTQPPSVSVSPGQTASIT | 71 |
| | CSGDKLGKKYSFWFQQKPGQSPVLVIYHDAKRPSGIPERF | |
| | SGSNSGDTATLTISGTQAMDEADYYCQAWKASTYVFGTGT | |
| | KVTVLGQPKAAPSVTLFPPSSEELQANKATLVCLISDFYP | |
| | GAVTVAWKADSSPVKAGVETTTPSKQSNNKYAASSYLSLT | |
| | PEQWKSHRSYSCQVTHEGSTVEKTVAPTECS | |
| 12345-1 H chain | MDMRVPAQLLGLLLLWLRGARCEVQLLESGGGLVQPGGSL | 72 |
| | RLSCAASGFTFSSYAMSWVRQAPGKGLEWVSAFSGWDVST | |
| | YYADAVKGRFTISRDNSKNTLYLQMNSLRAEDTAVYYCVK | |
| | DGLGPYFYNYGMDVWGQGTTVTVSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVD | |
| | KKVEPKSCDKTHTCPPCPAPELLGGPSVFLFPPKPKDTLM | |
| | ISRTPEVTCVVVDVSHEDPEVKFNWYVDGVEVHNAKTKPC | |
| | EEQYGSTYRCVSVLTVLHQDWLNGKEYKCKVSNKALPAPI | |
| | EKTISKAKGQPREPQVYTLPPSREEMTKNQVSLTCLVKGF | |
| | YPSDIAVEWESNGQPENNYKTTPPVLDSDGSFFLYSKLTV | |
| | DKSRWQQGNVFSCSVMHEALHNHYTQKSLSLSPGK | |
| 12345-1 L chain | MDMRVPAQLLGLLLLWLRGARCSYELTQPPSVSVSPGQTA | 73 |
| | SITCSGDKLGDKYSFWFQQKPGQSPVLVIYHDAKRPSGIP | |
| | ERFSGSNSGDTATLTISGTRAMDEADYYCQAWDSSTYVFG | |
| | TGTKVTVLGOPKAAPSVTLFPPSSEELQANKATLVCLISD | |
| | FYPGAVTVAWKADSSPVKAGVETTTPSKQSNNKYAASSYL | |
| | SLTPEQWKSHRSYSCQVTHEGSTVEKTVAPTECS | |

TABLE 23
CDR amino acid sequences

| | LCDR | SEQ | HCDR | SEQ |
|---|---|---|---|---|
| Antibody | amino acid | ID | amino acid | ID |
| Name | sequence | NO | sequence | NO |
| 8107-5 | | | | |
| CDR1 | SGDKLGDKYAF | 11 | SYAGS | 8 |
| CDR2 | HDSKRPS | 12 | AFSGSGGSTYYADSVKG | 106 |
| CDR3 | QAWDSSTYV | 13 | DGLGPYFYNYGMDV | 10 |
| 27461-1 | | | | |
| CDR1 | SGDKLGDKYAF | 11 | SYAGS | 8 |
| CDR2 | HDSKRPS | 12 | AFSGSGGSTYYADAVKG | 83 |
| CDR3 | QAWDSSTYV | 13 | DGLGPYFYNYGMDV | 10 |
| 27563-1 | | | | |
| CDR1 | SGDKLGDKYAF | 11 | SYAGS | 8 |
| CDR2 | HDSKRPS | 12 | AFSGSGGSTYYADAVKG | 83 |
| CDR3 | QAWDSSTYV | 13 | DGLGPYFYNYGMDV | 10 |
| 29351-1 | | | | |
| CDR1 | SGDKLGDKYSF | 74 | SYAMS | 107 |
| CDR2 | HDSKRPS | 12 | AFSGWDVSTYYADSVKG | 85 |
| CDR3 | QAWDSSTYV | 76 | DGLGPYFYNYGMDV | 10 |
| 28886-2 | | | | |
| CDR1 | SGDKLGDKYSF | 77 | SYAMS | 107 |
| CDR2 | HDSKRPS | 12 | AFSGWDVSTYYADAVKG | 85 |
| CDR3 | QAWDASTYV | 78 | DGLGPYFYNYGMDV | 10 |
| 29355-1 | | | | |
| CDR1 | SGKKLGKKYSF | 79 | SYAMS | 107 |
| CDR2 | HDSKRPS | 12 | AFSGWDVSTYYADAVKG | 9 |
| CDR3 | QAWKASTYV | 78 | DGLGPYFYNYGMDV | 10 |
| 29354-1 | | | | |
| CDR1 | SGKKLGKKYSF | 79 | SYAMS | 107 |
| CDR2 | HDAKRPS | 80 | AFSGWDVSTYYADAVKG | 85 |
| CDR3 | QAWDASTYV | 78 | DGLGPYFYNYGMDV | 10 |
| 29357-1 | | | | |
| CDR1 | SGDKLGKKYSF | 81 | SYAMS | 107 |
| CDR2 | HDAKRPS | 80 | AFSGWDVSTYYADAVKG | 85 |
| CDR3 | QAWKASTYV | 78 | DGLGPYFYNYGMDV | 10 |
| 29366-1 | | | | |
| CDR1 | SGDKLGKKYSF | 81 | SYAMS | 107 |
| CDR2 | HDAKRPS | 80 | AFSGWDVSTYYADAVKG | 85 |
| CDR3 | QAWKASTYV | 78 | DGLGPYFYNYGMDV | 10 |
| 102356-1 | | | | |
| CDR1 | SGKKLGKKYSF | 82 | SYAMS | 107 |
| CDR2 | HDAKRPS | 80 | AFSGWDVSTYYADAVKG | 85 |
| CDR3 | QAWDSSTYV | 78 | DGLGPYFYNYGMDV | 10 |
| 81074 | | | | |
| CDR1 | SGKKLGKKYSF | 82 | SYAMS | 107 |
| CDR2 | HDAKRPS | 80 | AFSGWDVSTYYADAVKG | 85 |
| CDR3 | QAWDSSTYV | 78 | DGLGPYFYNYGMDV | 10 |

TABLE 24
High Affinity anti-IL-13 antibody VL and VH amino acid sequences

| Anti- | | SEQ ID |
|---|---|---|
| body | VL/VH Amino Acid Sequence | NO: |
| 8107-5 | | |
| VL | SYELTQPPSVSVSPGQTASITCSGDKLGDKYAFWFQQKPGQSPVLVIYHDSKRPSGIPERFSGSNSGDTATL | 86 |
| | TISGTQAMDEADYYCQAWDSSTYVFGTGTKVTVL | |
| VH | EVQLLESGGGLVQPGGSLRLSCAASGFTFSSYAGSWVRQAPGKGLEWVSAFSGSGGSTYYADSVKGRFTISR | 87 |
| | DNSKNTLYLQMNSLRAEDTAVYYCVQDGLGPYFYNYGMDVWGQGTTVTVSS | |
| 27461-1 | | |
| VL | SYELTQPPSVSVSPGQTASITCSGDKLGDKYAFWFQQKPGQSPVLVIYHDAKRPSGIPERFSGSNSGDTATL | 88 |
| | TISGTQAMDEADYYCQAWDSSTYVFGTGTKVTVL | |
| VH | EVQLLESGGGLVQPGGSLRLSCAASGFTFSSYAGSWVRQAPGKGLEWVSAFSGSGGSTYYADAVKGRFTISR | 89 |
| | DNSKNTLYLQMNSLRAEDTAVYYCVKDGLGPYFYNYGMDVWGQGTTVTVSS | |
| 27563-1 | | |
| VL | SYELTQPPSVSVSPGQTASITCSGDKLGDKYAFWFQQKPGQSPVLVIYHDAKRPSGIPERFSGSNSGDTATL | 90 |
| | TISGTQAMDEADYYCQAWDASTYVFGTGTKVTVL | |
| VH | EVQLLESGGGLVQPGGSLRLSCAASGFTFSSYAGSWVRQAPGKGLEWVSAFSGSGGSTYYADAVKGRFTISR | 91 |
| | DNSKNTLYLQMNSLRAEDTAVYYCVKDGLGPYFYNYGMDVWGQGTTVTVSS | |
| 29351-1 | | |
| VL | SYELTQPPSVSVSPGQTASITCSGDKLGDKYSFWFQQKPGQSPVLVIYHDSKRPSGIPERFSGSNSGDTATL | 92 |
| | TISGTRAMDEADYYCQAWDSSTYVFGTGTKVTVL | |
| VH | EVQLLESGGGLVQPGGSLRLSCAASGFTFSSYAMSWVRQAPGKGLEWVSAFSGWDVSTYYADSVKGRFTISR | 93 |
| | DNSKNTLYLQMNSLRAEDTAVYYCVQDGLGPYFYNYGMDVWGQGTTVTVSS | |
| 28886-2 | | |
| VL | SYELTQPPSVSVSPGQTASITCSGDKLGDKYSFWFQQKPGQSPVLVIYHDAKRPSGIPERFSGSNSGDTATL | 94 |
| | TISGTRAMDEADYYCQAWDASTYVFGTGTKVTVL | |
| VH | EVQLLESGGGLVQPGGSLRLSCAASGFTFSSYAMSWVRQAPGKGLEWVSAFSGWDVSTYYADAVKGRFTISR | 95 |
| | DNSKNTLYLQMNSLRAEDTAVYYCVKDGLGPYFYNYGMDVWGQGTTVTVSS | |
| 29355-1 | | |
| VL | SYELTQPPSVSVSPGQTASITCSGKKLGKKYSFWFQQKPGQSPVLVIYHDAKRPSGIPERFSGSNSGDTATL | 96 |
| | TISGTRAMDEADYYCQAWKASTYVFGTGTKVTVL | |
| VH | EVQLLESGGGLVQPGGSLRLSCAASGFTFSSYAMSWVRQAPGKGLEWVSAFSGWDVSTYYADAVKGRFTISR | 97 |
| | DNSKNTLYLQMNSLRAEDTAVYYCVKDGLGPYFYNYGMDVWGQGTTVTVSS | |
| 29354-1 | | |
| VL | SYELTQPPSVSVSPGQTASITCSGKKLGKKYSFWFQQKPGQSPVLVIYHDAKRPSGIPERFSGSNSGNTATL | 98 |
| | TISGTRAMDEADYYCQAWDASTYVFGTGTKVTVL | |
| VH | EVQLLESGGGLVQPGGSLRLSCAASGFTFSSYAMSWVRQAPGKGLEWVSAFSGWDVSTYYADAVKGRFTISR | 99 |
| | DNSKNTLYLQMNSLRAEDTAVYYCVKDGLGPYFYNYGMDVWGQGTTVTVSS | |
| 29357-1 | | |
| VL | SYELTQPPSVSVSPGQTASITCSGDKLGKKYSFWFQQKPGQSPVLVIYHDAKRPSGIPERFSGSNSGNTATL | 100 |
| | TISGTRAMDEADYYCQAWKASTYVFGTGTKVTVL | |
| VH | EVQLLESGGGLVQPGGSLRLSCAASGFTFSSYAMSWVRQAPGKGLEWVSAFSGWDVSTYYADAVKGRFTISR | 101 |
| | DNSKNTLYLQMNSLRAEDTAVYYCVKDGLGPYFYNYGMDVWGQGTTVTVSS | |
| 29366-1 | | |
| VL | SYVLTQPPSVSVSPGQTASITCSGDKLGKKYSFWFQQKPGQSPVLVIYHDAKRPSGIPERFSGSNSGDTATL | 102 |
| | TISGTRAMDEADYYCQAWKASTYVFGTGTKVTVL | |
| VH | EVQLLESGGGLVQPGGSLRLSCAASGFTFSSYAMSWVRQAPGKGLEWVSAFSGWDVSTYYADAVKGRFTISR | 103 |
| | DNSKNTLYLQMNSLRAEDTAVYYCVKDGLGPYFYNYGMDVWGQGTTVTVSS | |
| 102356-1 | | |
| VL | SYELTQPPSVSVSPGQTASITCSGDKLGDKYSFWFQQKPGQSPVLVIYHDAKRPSGIPERFSGSNSGDTATL | 104 |
| | TISGTRAMDEADYYCQAWDSSTYVFGTGTKVTVL | |
| VH | EVQLLESGGGLVQPGGSLRLSCAASGFTFSSYAMSWVRQAPGKGLEWVSAFSGWDVSTYYADAVKGRFTISR | |
| | DNSKNTLYLQMNSLRAEDTAVYYCVKDGLGPYFYNYGMDVWGQGTTVTVSS | |

### Example 8: Use of Anti-IL-13 Antibodies as a Diagnostic Agents for Detection of IL-13 in a Sample

An Enzyme-Linked Immunosorbent Assay (ELISA) for the detection of IL-13 in a sample may be developed. In the assay, wells of a microtiter plate, such as a 96-well microtiter plate or a 384-well microtiter plate, are adsorbed for several hours with a first fully human monoclonal antibody directed against IL-13. The immobilized antibody serves as a capture antibody for IL-13 that may be present in a test sample. The wells are rinsed and treated with a blocking agent such as milk protein or albumin to prevent nonspecific adsorption of the analyte.

Subsequently the wells are treated with a test sample suspected of containing IL-13, or with a solution containing a standard amount of the antigen.

After rinsing away the test sample or standard, the wells are treated with a second fully human monoclonal anti-IL-13 antibody that is labeled by conjugation with biotin. The labeled anti-IL-13 antibody serves as a detecting antibody. After rinsing away excess second antibody, the wells are treated with avidin-conjugated horseradish peroxidase (HRP) and a suitable chromogenic substrate. The concentration of the antigen in the test samples is determined by comparison with a standard curve developed from the standard samples.

### Example 9: Treatment of COPD in Humans

A patient suffering from COPD is identified. The patient receives an effective amount of the anti-IL-13 antibodies disclosed above is administered by intravenous or subcutaneous injection to the patient. A booster administration is given three weeks later, and every three weeks thereafter. The anti-IL-13 antibody causes an inhibition in the production of mucous, the development of bronchial epithelium hyperplasia, and spasm of bronchial smooth muscle. This inhibition of mucous production and smooth muscle contraction reduces blockade of air passage with improved ventilation.

### Example 10: Treatment of Chronic Bronchitis in Humans

A patient suffering from chronic obstruction pulmonary disease (“COPD”) characterized by chronic bronchitis is identified. The patient receives an effective amount of the anti-IL-13 antibody disclosed herein, by intravenous or subcutaneous injection to the patient. Treatments can be repeated every week, or every two weeks, or every three weeks, or four weeks, or monthly, or every other month The anti-IL-13 antibody causes a partial or complete inhibition of mucous production and bronchial smooth muscle contraction in the inflamed respiratory tissues. This inhibition of mucous production and smooth muscle contraction reduces blockade of air passage with improved ventilation.

### Example 11: Treatment of Emphysema in Humans

A patient suffering from emphysema is identified. The patient receives an effective amount of the IL-13 antibody by intravenous or subcutaneous injection to the patient Treatments can be repeated every week, or every two weeks, or every three weeks, or four weeks, or monthly, or every other month The IL-13 antibody causes a partial or complete inhibition of neutrophil chemotaxis in the inflamed respiratory tissues. This inhibition of neutrophil chemotaxis reduces the severity of tissue damage to the lungs and air passages caused by the patient's immune response. There is a direct action of IL-13 in the induction of proteases that lead to the destruction of lung tissue in emphysema (at least that is hypothesized).

### Example 12: Treatment of Asthma in Humans

A patient suffering from asthma is identified. The patient receives an effective amount of the IL-13 antibody by intravenous or subcutaneous injection to the patient.

Treatments can be repeated every week, or every two weeks, or every three weeks, or four weeks, or monthly, or every other month. The anti-IL-13 antibody reduces the severity of tissue damage to the lungs and air passages caused by the patient's immune response.

### Example 13: Treatment of Atopic Dermatitis in Humans

A patient suffering from atopic dermatitis is identified. The patient receives an effective amount of the IL-13 antibody by intravenous or subcutaneous injection to the patient. Treatments can be repeated every week, or every two weeks, or every three weeks, or four weeks, or monthly, or every other month.

### Example 14: Optimized Sequences for High Affinity Anti IL-13 Antibodies

Engineering of MmAb5/MmAb7 hotspots began with an in-silico scan with internal software for chemical liabilities and consensus violations from germline sequences. Results revealed four potential isomerization sites in CDRs (Asp Ser and Asp Gly), one consensus violation, and one potential Trp oxidation site in a CDR. To assess potential mutations to mitigate risk, an internally derived co-crystal structure of MmAb7 and IL-13 was analyzed for exposure and interaction of hotspot residues. From the structure, it was observed that on LC: DS 67-68, the Asp residue was directly interacting with a positively charged residue on the antigen. To retain that interaction, the fix was limited to changing the Ser residue, and an Ala substitution was chosen based on internal experience. At LC: DS 110-111, the Asp was observed to interact with a positively charged residue on an adjacent CDR, providing structure. To retain that function in the molecule, Asp was left alone and DS was changed to DA. At HC: DS 72-73, the isomerization site was found to be exposed on the surface of the molecule and non-interactive. For improved homogeneity, DS was changed to DA. The HC: DG 109-110 site was observed to be buried within the structure and not subject to isomerization. A G110A variant was only tested individually and was found to have lost activity. At HC: Q108, the residue was changed to germline. Hotspot fixes at the various sites were tested individually and in combination using a rational design. Selection of lead variants was based on production yield, Tm, and functional activity using the TARC and Eotaxin assays.

Engineering of MmAb5 and MmAb7 viscosity began with an in-silico surface analysis of the MmAb7/IL-13 co-crystal structure using BioLuminate Schrodinger software in which charge patches and contributing residues were identified. From the most prominent charge patch, key contributing residues were analyzed for potential antigen binding and structural impact. The LC: D3 residue was substituted with Val from an alternate germline. The LC: D87 residue was substituted with Asn from an alternate germline. Residues at L:D26, L:D33, and L:D110 were substituted with Lys for optimal patch disruption. Viscosity fixes at the various sites were tested individually and in combination using a rational design. Selection of lead variants was based on production yield, viscosity measured by cone and plate methods, Tm, and functional activity using the TARC assay.

The TARC assay measures inhibition TARC generated by IL-13 sensitive progenitor cells in the presence of IL-13. Anti-IL-13 mAbs being measured for TARC inhibition are serially diluted prior to being added to a set amount of IL-13 (3 ng/mL) and incubated for 20 minutes at room temperature. After incubation, the mAb and IL-13 solutions are added to 2e5 cells in a 96-well tissue culture plate and incubated at 37 C and 5% C02 for 48 hours. After incubation, samples are collected and TARC is measured using and anti-TARC mAb from a detection kit by MSD. The plate is read using MSD 6000. Dose response data is analyzed to generate dose response curves and calculate IC50 levels using Graph Pad Prism software.

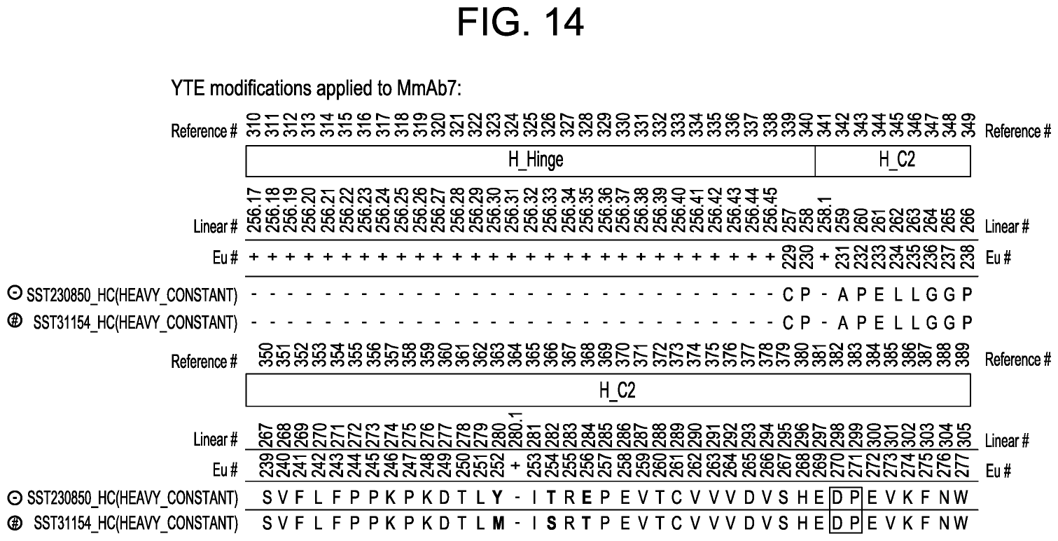

Variants of anti-IL-13 monoclonal antibodies will also include half life extension mutations in the Fc at Eu positions M252Y, S254T, and T256E, known commonly as YTE mutations. These modifications improve FcRn binding with the effect of antibodies being recycled back into circulation after endocytosis by effector function cells. These modifications are being used with the intent of extending PK as well as decreasing dose requirements and/or dosing frequency. See FIG. 14.

Variants of anti-IL-13 monoclonal antibodies will also include a complement hexamer disrupting mutation in the Fc at Eu position S583K. By hindering the formation of mAb hexamers in as part of the complex effector function mechanism, his modification has been observed to decrease viscosity of an antibody in a given formulation and concentration.

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed:

1. An antigen binding protein that specifically binds to human IL-13 comprising a light chain immunoglobulin variable region (VL1) and a heavy chain immunoglobulin variable region (VH),
wherein VL1 comprises (i) a CDRL1 comprising an amino acid sequence of SEQ ID NO: 11; (ii) a CDRL2 comprising an amino acid sequence of SEQ ID NO: 12, and (iii) a CDRL3 comprising an amino acid of SEQ ID NO: 13;
and VH1 comprising an amino acid sequence of (i) comprising an amino acid sequence of SEQ ID NO: 8 (ii) a CDRH2 comprising an amino acid sequence of SEQ ID NO: 9, and (iii) a CDRH3 comprising an amino acid sequence of SEQ ID NO: 10.

2. An antigen binding protein that specifically binds to human IL-13 comprising a light chain immunoglobulin variable region (VL1) and a heavy chain immunoglobulin variable region (VH),
wherein VL comprises the CDRs of the antibody expressed by cell 623,
and VH comprises the CDRS pf the antibody expressed by cell 623.

3. The antigen binding proteins of claim 1 further comprising the framework regions as the antibody expressed by cell 623.

4. The antigen binding protein of claims 1-3 wherein the antigen binding protein is an antibody.

5. The antigen binding protein of claims 1-3 wherein the antigen binding protein is an antibody fragment.

6. The antigen binding protein of claims 1-3 wherein the antigen binding protein is an antibody derivative comprising a bispecific antibody, a fusion protein.

7. The antigen binding protein of claims 1-6 wherein the antigen binding protein has human sequences.

8. The antigen binding protein of claims 1-6 wherein the antigen binding is a monoclonal antibody.

9. A human antibody that binds to IL-13, wherein the human antibody binds to IL-13 with a K_{D} of between 2 cM to 50pM.

10. A human antibody that binds to IL-13, wherein the human antibody binds to IL-13 with a K_{D} of 2 cM to 40pM.

11. A human antibody or antigen binding fragment thereof that binds to human IL-13 selected from the group wherein the amino acid sequences comprise
(a) an antibody variable light chain amino acid sequence comprising LCDR1 of SEQ ID NO:11, a LCDR2 of SEQ ID NO:12, and a LCDR3 of SEQ ID NO: 13; and an variable antibody heavy chain amino acid sequence comprising HCDR1 of SEQ ID NO: 8, a HCDR2 of SEQ ID NO: 106 and a HCDR3 of SEQ ID NO:10;
(b) an antibody variable light chain amino acid sequence comprising LCDR1 of SEQ ID NO:11, a LCDR2 of SEQ ID NO: 12, and a LCDR3 of SEQ ID NO: 13; and an variable antibody heavy chain amino acid sequence comprising HCDR1 of SEQ ID NO: 8, a HCDR2 of SEQ ID NO: 83; and a HCDR3 of SEQ ID NO: 10;
(c) an antibody variable light chain amino acid sequence comprising LCDR1 of SEQ ID NO:11, a LCDR2 of SEQ ID NO:12, and a LCDR3 of SEQ ID NO: 13; and an variable antibody heavy chain amino acid sequence comprising HCDR1 of SEQ ID NO: 8, a HCDR2 of SEQ ID NO: 83, and a HCDR3 of SEQ ID NO:10;
(d) an antibody variable light chain amino acid sequence comprising LCDR1 of SEQ ID NO: 74, a LCDR2 of SEQ ID NO: 12, and a LCDR3 of SEQ ID NO: 76; and an variable antibody heavy chain amino acid sequence comprising HCDR1 of SEQ ID NO: 107, a HCDR2 of SEQ ID NO: 85, and a HCDR3 of SEQ ID NO: 10;
(e) an antibody variable light chain amino acid sequence comprising LCDR1 of SEQ ID NO: 77, a LCDR2 of SEQ ID NO: 12, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO: 10;
(f) an antibody variable light chain amino acid sequence comprising LCDR1 of SEQ ID NO: 79, a LCDR2 of SEQ ID NO: 12, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO: 10;
(g) an antibody variable light chain amino acid sequence comprising LCDR1 of SEQ ID NO: 79, a LCDR2 of SEQ ID NO: 80, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO:10;
(h) an antibody variable light chain amino acid sequence comprising LCDR1 of SEQ ID NO: 81, a LCDR2 of SEQ ID NO: 80, and a LCDR3 of SEQ ID NO: 78; and an variable antibody heavy chain amino acid sequence comprising HCDR1 of SEQ ID NO: 107, a HCDR2 of SEQ ID NO: 85, and a HCDR3 of SEQ ID NO:10; and
(i) an antibody variable light chain amino acid sequence comprising LCDR1 of SEQ ID NO: 82, a LCDR2 of SEQ ID NO: 80, and a LCDR3 of SEQ ID NO: 78; and an antibody variable heavy chain amino acid sequence comprising HCDR1 of SEQ ID NO:107, a HCDR2 of SEQ ID NO: 85, and a HCDR3 of SEQ ID NO: 10.

12. A human antibody or antigen binding fragment thereof that binds to human IL-13 wherein the amino acid sequences comprise a variable light chain region and a variable heavy chain region selected from the group comprising
(a) an antibody variable light chain amino acid sequence comprising SEQ ID NO: 86 and an antibody variable heavy chain amino acid sequence comprising SEQ ID NO: 87;
(b) an antibody variable light chain amino acid sequence comprising SEQ ID NO: 88 and an antibody variable heavy chain amino acid sequence comprising SEQ ID NO: 89;
(c) an antibody variable light chain amino acid sequence comprising SEQ ID NO: 90 and an antibody variable heavy chain amino acid sequence comprising SEQ ID NO: 91;
(d) an antibody variable light chain amino acid sequence comprising SEQ ID NO: 92 and an antibody variable heavy chain amino acid sequence comprising SEQ ID NO: 93;
(e) an antibody variable light chain amino acid sequence comprising SEQ ID NO: 94 and an antibody variable heavy chain amino acid sequence comprising SEQ ID NO: 95;
(f) an antibody variable light chain amino acid sequence comprising SEQ ID NO: 96 and an antibody variable heavy chain amino acid sequence comprising SEQ ID NO: 97;
(g) an antibody variable light chain amino acid sequence comprising SEQ ID NO: 98 and an antibody variable heavy chain amino acid sequence comprising SEQ ID NO: 99;
(h) an antibody variable light chain amino acid sequence comprising SEQ ID NO: 100 and an antibody variable heavy chain amino acid sequence comprising SEQ ID NO: 101;
(i) an antibody variable light chain amino acid sequence comprising SEQ ID NO: 102 and an antibody variable heavy chain amino acid sequence comprising SEQ ID NO: 103, and
(j) an antibody variable light chain amino acid sequence comprising SEQ ID NO: 104 and an antibody variable heavy chain amino acid sequence comprising SEQ ID NO: 105.

13. A human antibody or antigen binding fragment thereof that binds to human IL-13 wherein the amino acid sequences
(a) comprises a light chain selected from the group comprising SEQ ID NO: 22, SEQ ID NO: 24, SEQ ID NO: 26, SEQ ID NO: 28, SEQ ID NO: 30, SEQ ID NO: 33, SEQ ID NO: 35, SEQ ID NO: 37; SEQ ID NO: 39, SEQ ID NO: 41; SEQ ID NO: 43, SEQ ID NO: 45, SEQ ID NO: 47, SEQ ID NO: 49, SEQ ID NO: 51, SEQ ID NO: 54, SEQ ID NO: 56, SEQ ID NO: 58, SEQ ID NO: 60, SEQ ID NO: 62, SEQ ID NO: 65, SEQ ID NO: 67, SEQ ID NO: 69, SEQ ID NO: 71, and SEQ ID NO:73, and
(b) a heavy chain selected from the group comprising SEQ ID NO: 23, SEQ ID NO: 25, SEQ ID NO: 27, SEQ ID NO: 29, SEQ ID NO: 31, SEQ ID NO: 32, SEQ ID NO: 34, SEQ ID NO: 36; SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 53, SEQ ID NO: 55, SEQ ID NO: 57, SEQ ID NO: 59, SEQ ID NO: 61, SEQ ID NO: 63, SEQ ID NO: 64, SEQ ID NO: 66, SEQ ID NO: 68, SEQ ID NO: 70, and SEQ ID NO: 72.

14. An antibody of comprising a light chain and a heavy chain having the amino acid sequences claims 1-14, 20, 25-27.

15. A nucleic acid sequence encoding an antibody or antibody fragment thereof claims 1-14

16. A vector comprising the nucleic acid sequence encoding an antibody or antibody fragment thereof claim 15, 20, 25-27

17. A host cell comprising the vector of claim 16.

18. The host cell of claim 17 wherein the host cell is a CHO cell or a SP2/0 cell.

19. The host cell of claim 18 wherein the host cell is a CHO cell.

20. An antibody or antibody fragment produced by the host cell of claims 18-19.

21. A pharmaceutical composition comprising the antibody or antibody binding fragment of claim 20.

22. A method of producing an antibody or fragment thereof by culturing the host cells of claims 17-19.

23. A method of treating a patient suffering from COPD, emphysema, asthma, or atopic dermatitis by administering and effective amount of the antibodies or fragments thereof of claim 20 to the patient.

24. A method of treating a patient suffering from COPD, emphysema, asthma, or atopic dermatitis by administering and effective amount of the pharmaceutical composition of claim 21 to the patient.

25. The antibodies of claim 20 wherein there is a half-life extension mutation.

26. The antibodies of claim 25 wherein the half-life extension mutation is a mutation in the Fc at Eu positions M252Y, S254T, and T256E.

27. The antibodies of claim 25 wherein the half-life extension mutation is a mutation in the Fc at complement hexamer disrupting mutation in the Fc at Eu position S583K.


SEQUENCE LISTING (223 sequences)

SEQ ID NO: 1 - protein, 112 aa, Artificial Sequence
  Description of Artificial Sequence: Synthetic polypeptide
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 2 - protein, 114 aa, Artificial Sequence
  Description of Artificial Sequence: Synthetic polypeptide
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 3 - protein, 115 aa, Artificial Sequence
  Description of Artificial Sequence: Synthetic polypeptide
MSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 4 - protein, 364 aa, Artificial Sequence [sequence omitted]

SEQ ID NO: 5 - protein, 367 aa, Artificial Sequence
  Description of Artificial Sequence: Synthetic polypeptide
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNRYLDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 6 - protein, 150 aa, Artificial Sequence
  Description of Artificial Sequence: Synthetic polypeptide
MHPLLNPLLLALGLMALLLTTVIALTCLGGFASPGPVPRSVSLPLTLKELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 7 - protein, 146 aa, Artificial Sequence
  Description of Artificial Sequence: Synthetic polypeptide
MHPLLNPLLLALGLMALLLTTVIALTCLGGFASPGPVPPSTALRELIEELVNITQNQKAPLCNGSMVWSINLTAGGFCVALDSLTNVSGCSAIEKTQRMLSGFCPHKVSAGQFSSLHVRDTKIEVAQFVKDLLLHLKKLFREGQFN

SEQ ID NO: 8 - protein, 5 aa, Artificial Sequence
  Description of Artificial Sequence: Synthetic peptide
SYAGS

SEQ ID NO: 9 - protein, 17 aa, Artificial Sequence
  Description of Artificial Sequence: Synthetic peptide
AFSGWDVSTYYADSVKG

SEQ ID NO: 10 - protein, 14 aa, Artificial Sequence
  Description of Artificial Sequence: Synthetic peptide
DGLGPYFYNYGMDV

SEQ ID NO: 11 - protein, 10 aa, Artificial Sequence
  Description of Artificial Sequence: Synthetic peptide
SGDKLGDKYS

SEQ ID NO: 12 - protein, 7 aa, Artificial Sequence
  Description of Artificial Sequence: Synthetic peptide
HDSKRPS

SEQ ID NO: 13 - protein, 9 aa, Artificial Sequence
  Description of Artificial Sequence: Synthetic peptide
QAWDSSTYV

SEQ ID NO: 14 - protein, 88 aa, Artificial Sequence
  Description of Artificial Sequence: Synthetic polypeptide
GPVPPSTALRELIEELVNITQNQKAPLCNGSMVWSINLTAGMYCAALESLINVSGCSAIEKTQRMLSGFCPHKVSAGQFSSLHVRDTK

SEQ ID NO: 15 - protein, 73 aa, Artificial Sequence
  Description of Artificial Sequence: Synthetic polypeptide
GPVPPSTALRELIEELVNITQNQKAPLCNGSMVWSINLTAGMYCAALESLINVSGCSAIEKTQRMLSGFCPHK

SEQ ID NO: 16 - protein, 72 aa, Artificial Sequence
  Description of Artificial Sequence: Synthetic polypeptide
APLCNGSMVWSINLTAGMYCAALESLINVSGCSAIEKTQRMLSGFCPHKVSAGQFSSLHVRDTKIEVAQFVK

SEQ ID NO: 17 - protein, 64 aa, Artificial Sequence
  Description of Artificial Sequence: Synthetic polypeptide
APLCNGSMVWSINLTAGMYCAALESLINVSGCSAIEKTQRMLSGFCPHKVSAGQFSSLHVRDTK

SEQ ID NO: 18 - protein, 150 aa, Artificial Sequence; identical to SEQ ID NO: 6
  Description of Artificial Sequence: Synthetic polypeptide

SEQ ID NO: 19 - protein, 146 aa, Artificial Sequence; identical to SEQ ID NO: 7
  Description of Artificial Sequence: Synthetic polypeptide

SEQ ID NO: 20 - protein, 146 aa, Artificial Sequence
  Description of Artificial Sequence: Synthetic polypeptide
MHPLLNPLLLALGLMALLLTTVIALTCLGGFASPGPVPPSTALRELIEELVNITQNQKAPLCNGSMVWSINLTAGMYCAALESLINVSGCSAIYRTQRILHGLCPHKVSAGQFSSLHVRDTKIEVAQFVKDLLLHLKKLFREGQFN

SEQ ID NO: 21 - protein, 147 aa, Artificial Sequence
  Description of Artificial Sequence: Synthetic polypeptide
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAHFITKLLSYTKQLFRHGQQFN

SEQ ID NO: 22 - protein, 231 aa, Artificial Sequence
  Description of Artificial Sequence: Synthetic polypeptide
MAWALLLLTLLTQGTGSWASYELTQPPSVSVSPGQTASITCSGDKLGDKYAFWFQQKPGQSPVLVIYHDAKRPSGIPERFSGSNSGDTATLTISGTQAMDEADYYCQAWDSSTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAGSWVRQAPGKGLEWVSAFSGSGGSTYYADSVKGRFTISRDNSKNTLYLQMNSLRAEDTAVYYCVQDGLGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 231 aa, Artificial Sequence
  Description of Artificial Sequence: Synthetic polypeptide
MAWALLLLTLLTQGTGSWASYELTQPPSVSVSPGQTASITCSGDKLGDKYAFWFQQKPGQSPVLVIYHDSKRPSGIPERFSGSNSGDTATLTISGTQAMDEADYYCQAWDASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 475 aa, Artificial Sequence; identical to SEQ ID NO: 23
  Description of Artificial Sequence: Synthetic polypeptide

SEQ ID NO: 26 - protein, 231 aa, Artificial Sequence
  Description of Artificial Sequence: Synthetic polypeptide
MAWALLLLTLLTQGTGSWASYELTQPPSVSVSPGQTASITCSGDKLGDKYAFWFQQKPGQSPVLVIYHDAKRPSGIPERFSGSNSGDTATLTISGTQAMDEADYYCQAWDASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - protein, 475 aa, Artificial Sequence; identical to SEQ ID NO: 23
  Description of Artificial Sequence: Synthetic polypeptide

SEQ ID NO: 28 - protein, 231 aa, Artificial Sequence
  Description of Artificial Sequence: Synthetic polypeptide
MAWALLLLTLLTQGTGSWASYELTQPPSVSVSPGQTASITCSGDKLGDKYAFWFQQKPGQSPVLVIYHDSKRPSGIPERFSGSNSGDTATLTISGTQAMDEADYYCQAWDSSTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAGSWVRQAPGKGLEWVSAFSGSGGSTYYADAVKGRFTISRDNSKNTLYLQMNSLRAEDTAVYYCVQDGLGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 231 aa, Artificial Sequence; identical to SEQ ID NO: 28
  Description of Artificial Sequence: Synthetic polypeptide

SEQ ID NO: 31 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAGSWVRQAPGKGLEWVSAFSGSGGSTYYADSVKGRFTISRDNSKNTLYLQMNSLRAEDTAVYYCVQDALGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 475 aa, Artificial Sequence; identical to SEQ ID NO: 31
  Description of Artificial Sequence: Synthetic polypeptide

SEQ ID NO: 33 - protein, 231 aa, Artificial Sequence; identical to SEQ ID NO: 28
  Description of Artificial Sequence: Synthetic polypeptide

SEQ ID NO: 34 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAGSWVRQAPGKGLEWVSAFSGSGGSTYYADSVKGRFTISRDNSKNTLYLQMNSLRAEDTAVYYCVKDGLGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 231 aa, Artificial Sequence; identical to SEQ ID NO: 22
  Description of Artificial Sequence: Synthetic polypeptide

SEQ ID NO: 36 - protein, 475 aa, Artificial Sequence; identical to SEQ ID NO: 29
  Description of Artificial Sequence: Synthetic polypeptide

SEQ ID NO: 37 - protein, 231 aa, Artificial Sequence; identical to SEQ ID NO: 24
  Description of Artificial Sequence: Synthetic polypeptide

SEQ ID NO: 38 - protein, 475 aa, Artificial Sequence; identical to SEQ ID NO: 29
  Description of Artificial Sequence: Synthetic polypeptide

SEQ ID NO: 39 - protein, 231 aa, Artificial Sequence; identical to SEQ ID NO: 26
  Description of Artificial Sequence: Synthetic polypeptide

SEQ ID NO: 40 - protein, 475 aa, Artificial Sequence; identical to SEQ ID NO: 29
  Description of Artificial Sequence: Synthetic polypeptide

SEQ ID NO: 41 - protein, 231 aa, Artificial Sequence; identical to SEQ ID NO: 22
  Description of Artificial Sequence: Synthetic polypeptide

SEQ ID NO: 42 - protein, 475 aa, Artificial Sequence; identical to SEQ ID NO: 34
  Description of Artificial Sequence: Synthetic polypeptide

SEQ ID NO: 43 - protein, 231 aa, Artificial Sequence; identical to SEQ ID NO: 26
  Description of Artificial Sequence: Synthetic polypeptide

SEQ ID NO: 44 - protein, 475 aa, Artificial Sequence; identical to SEQ ID NO: 34
  Description of Artificial Sequence: Synthetic polypeptide

SEQ ID NO: 45 - protein, 231 aa, Artificial Sequence; identical to SEQ ID NO: 22
  Description of Artificial Sequence: Synthetic polypeptide

SEQ ID NO: 46 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAGSWVRQAPGKGLEWVSAFSGSGGSTYYADAVKGRFTISRDNSKNTLYLQMNSLRAEDTAVYYCVKDGLGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 231 aa, Artificial Sequence; identical to SEQ ID NO: 24
  Description of Artificial Sequence: Synthetic polypeptide

SEQ ID NO: 48 - protein, 475 aa, Artificial Sequence; identical to SEQ ID NO: 46
  Description of Artificial Sequence: Synthetic polypeptide

SEQ ID NO: 49 - protein, 231 aa, Artificial Sequence; identical to SEQ ID NO: 26
  Description of Artificial Sequence: Synthetic polypeptide

SEQ ID NO: 50 - protein, 475 aa, Artificial Sequence; identical to SEQ ID NO: 46
  Description of Artificial Sequence: Synthetic polypeptide

SEQ ID NO: 51 - protein, 231 aa, Artificial Sequence; identical to SEQ ID NO: 24
  Description of Artificial Sequence: Synthetic polypeptide

SEQ ID NO: 52 - protein, 475 aa, Artificial Sequence; identical to SEQ ID NO: 34
  Description of Artificial Sequence: Synthetic polypeptide

SEQ ID NO: 53 - protein, 475 aa, Artificial Sequence; identical to SEQ ID NO: 23
  Description of Artificial Sequence: Synthetic polypeptide

SEQ ID NO: 54 - protein, 231 aa, Artificial Sequence; identical to SEQ ID NO: 28
  Description of Artificial Sequence: Synthetic polypeptide

SEQ ID NO: 55 - protein, 475 aa, Artificial Sequence; identical to SEQ ID NO: 46
  Description of Artificial Sequence: Synthetic polypeptide

SEQ ID NO: 56 - protein, 231 aa, Artificial Sequence; identical to SEQ ID NO: 22
  Description of Artificial Sequence: Synthetic polypeptide

SEQ ID NO: 57 - protein, 475 aa, Artificial Sequence; identical to SEQ ID NO: 46
  Description of Artificial Sequence: Synthetic polypeptide

SEQ ID NO: 58 - protein, 231 aa, Artificial Sequence; identical to SEQ ID NO: 26
  Description of Artificial Sequence: Synthetic polypeptide

SEQ ID NO: 59 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAMSWVRQAPGKGLEWVSAFSGWDVSTYYADSVKGRFTISRDNSKNTLYLQMNSLRAEDTAVYYCVQDGLGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 234 aa, Artificial Sequence
  Description of Artificial Sequence: Synthetic polypeptide
MDMRVPAQLLGLLLLWLRGARCSYELTQPPSVSVSPGQTASITCSGDKLGDKYSFWFQQKPGQSPVLVIYHDSKRPSGIPERFSGSNSGDTATLTISGTRAMDEADYYCQAWDSSTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 61 - protein, 475 aa, Artificial Sequence
  Description of Artificial Sequence: Synthetic polypeptide
MDMRVPAQLLGLLLLWLRGARCEVQLLESGGGLVQPGGSLRLSCAASGFTFSSYAMSWVRQAPGKGLEWVSAFSGWDVSTYYADAVKGRFTISRDNSKNTLYLQMNSLRAEDTAVYYCVKDGLGPYFYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 231 aa, Artificial Sequence
  Description of Artificial Sequence: Synthetic polypeptide
MAWALLLLTLLTQGTGSWASYELTQPPSVSVSPGQTASITCSGDKLGDKYSFWFQQKPGQSPVLVIYHDAKRPSGIPERFSGSNSGDTATLTISGTQAMDEADYYCQAWDASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 63 - protein, 475 aa, Artificial Sequence; identical to SEQ ID NO: 61
  Description of Artificial Sequence: Synthetic polypeptide

SEQ ID NO: 64 - protein, 475 aa, Artificial Sequence; identical to SEQ ID NO: 61
  Description of Artificial Sequence: Synthetic polypeptide

SEQ ID NO: 65 - protein, 231 aa, Artificial Sequence
  Description of Artificial Sequence: Synthetic polypeptide
MAWALLLLTLLTQGTGSWASYELTQPPSVSVSPGQTASITCSGKKLGKKYSFWFQQKPGQSPVLVIYHDAKRPSGIPERFSGSNSGNTATLTISGTQAMDEADYYCQAWDASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 231 aa, Artificial Sequence
  Description of Artificial Sequence: Synthetic polypeptide
MAWALLLLTLLTQGTGSWASYELTQPPSVSVSPGQTASITCSGKKLGKKYSFWFQQKPGQSPVLVIYHDAKRPSGIPERFSGSNSGDTATLTISGTQAMDEADYYCQAWKASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 231 aa, Artificial Sequence; identical to SEQ ID NO: 66
  Description of Artificial Sequence: Synthetic polypeptide

SEQ ID NO: 68 - protein, 475 aa, Artificial Sequence; identical to SEQ ID NO: 61
  Description of Artificial Sequence: Synthetic polypeptide

SEQ ID NO: 69 - protein, 231 aa, Artificial Sequence
  Description of Artificial Sequence: Synthetic polypeptide
MAWALLLLTLLTQGTGSWASYELTQPPSVSVSPGQTASITCSGDKLGKKYSFWFQQKPGQSPVLVIYHDAKRPSGIPERFSGSNSGNTATLTISGTQAMDEADYYCQAWKASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 475 aa, Artificial Sequence; identical to SEQ ID NO: 61
  Description of Artificial Sequence: Synthetic polypeptide

SEQ ID NO: 71 - protein, 231 aa, Artificial Sequence
  Description of Artificial Sequence: Synthetic polypeptide
MAWALLLLTLLTQGTGSWASYVLTQPPSVSVSPGQTASITCSGDKLGKKYSFWFQQKPGQSPVLVIYHDAKRPSGIPERFSGSNSGDTATLTISGTQAMDEADYYCQAWKASTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - protein, 475 aa, Artificial Sequence; identical to SEQ ID NO: 61
  Description of Artificial Sequence: Synthetic polypeptide

SEQ ID NO: 73 - protein, 234 aa, Artificial Sequence
  Description of Artificial Sequence: Synthetic polypeptide
MDMRVPAQLLGLLLLWLRGARCSYELTQPPSVSVSPGQTASITCSGDKLGDKYSFWFQQKPGQSPVLVIYHDAKRPSGIPERFSGSNSGDTATLTISGTRAMDEADYYCQAWDSSTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 11 aa, Artificial Sequence
  Description of Artificial Sequence: Synthetic peptide
SGDKLGDKYSF

SEQ ID NO: 75 - skipped

SEQ ID NO: 76 - protein, 9 aa, Artificial Sequence
  Description of Artificial Sequence: Synthetic peptide
QAWDSSTYV

SEQ ID NO: 77 - protein, 11 aa, Artificial Sequence
  Description of Artificial Sequence: Synthetic peptide
SGDKLGDKYSF

SEQ ID NO: 78 - protein, 9 aa, Artificial Sequence
  Description of Artificial Sequence: Synthetic peptide
QAWDASTYV

SEQ ID NO: 79 - protein, 11 aa, Artificial Sequence
  Description of Artificial Sequence: Synthetic peptide
SGKKLGKKYSF

SEQ ID NO: 80 - protein, 7 aa, Artificial Sequence
  Description of Artificial Sequence: Synthetic peptide
HDAKRPS

SEQ ID NO: 81 - protein, 11 aa, Artificial Sequence
  Description of Artificial Sequence: Synthetic peptide
SGDKLGKKYSF

SEQ ID NO: 82 - protein, 11 aa, Artificial Sequence
  Description of Artificial Sequence: Synthetic peptide
SGKKLGKKYSF

SEQ ID NO: 83 - protein, 17 aa, Artificial Sequence
  Description of Artificial Sequence: Synthetic peptide
AFSGSGGSTYYADAVKG

SEQ ID NO: 84 - skipped

SEQ ID NO: 85 - protein, 17 aa, Artificial Sequence
  Description of Artificial Sequence: Synthetic peptide
AFSGWDVSTYYADAVKG

SEQ ID NO: 86 - protein, 106 aa, Artificial Sequence
  Description of Artificial Sequence: Synthetic polypeptide
SYELTQPPSVSVSPGQTASITCSGDKLGDKYAFWFQQKPGQSPVLVIYHDSKRPSGIPERFSGSNSGDTATLTISGTQAMDEADYYCQAWDSSTYVFGTGTKVTVL

SEQ ID NO: 87 - protein, 123 aa, Artificial Sequence
  Description of Artificial Sequence: Synthetic polypeptide
EVQLLESGGGLVQPGGSLRLSCAASGFTFSSYAGSWVRQAPGKGLEWVSAFSGSGGSTYYADSVKGRFTISRDNSKNTLYLQMNSLRAEDTAVYYCVQDGLGPYFYNYGMDVWGQGTTVTVSS

SEQ ID NO: 88 - protein, 106 aa, Artificial Sequence
  Description of Artificial Sequence: Synthetic polypeptide
SYELTQPPSVSVSPGQTASITCSGDKLGDKYAFWFQQKPGQSPVLVIYHDAKRPSGIPERFSGSNSGDTATLTISGTQAMDEADYYCQAWDSSTYVFGTGTKVTVL

SEQ ID NO: 89 - protein, 123 aa, Artificial Sequence
  Description of Artificial Sequence: Synthetic polypeptide
EVQLLESGGGLVQPGGSLRLSCAASGFTFSSYAGSWVRQAPGKGLEWVSAFSGSGGSTYYADAVKGRFTISRDNSKNTLYLQMNSLRAEDTAVYYCVKDGLGPYFYNYGMDVWGQGTTVTVSS

SEQ ID NO: 90 - protein, 106 aa, Artificial Sequence
  Description of Artificial Sequence: Synthetic polypeptide
SYELTQPPSVSVSPGQTASITCSGDKLGDKYAFWFQQKPGQSPVLVIYHDAKRPSGIPERFSGSNSGDTATLTISGTQAMDEADYYCQAWDASTYVFGTGTKVTVL

SEQ ID NO: 91 - protein, 123 aa, Artificial Sequence; identical to SEQ ID NO: 89
  Description of Artificial Sequence: Synthetic polypeptide

SEQ ID NO: 92 - protein, 106 aa, Artificial Sequence
  Description of Artificial Sequence: Synthetic polypeptide
SYELTQPPSVSVSPGQTASITCSGDKLGDKYSFWFQQKPGQSPVLVIYHDSKRPSGIPERFSGSNSGDTATLTISGTRAMDEADYYCQAWDSSTYVFGTGTKVTVL

SEQ ID NO: 93 - protein, 123 aa, Artificial Sequence
  Description of Artificial Sequence: Synthetic polypeptide
EVQLLESGGGLVQPGGSLRLSCAASGFTFSSYAMSWVRQAPGKGLEWVSAFSGWDVSTYYADSVKGRFTISRDNSKNTLYLQMNSLRAEDTAVYYCVQDGLGPYFYNYGMDVWGQGTTVTVSS

SEQ ID NO: 94 - protein, 106 aa, Artificial Sequence
  Description of Artificial Sequence: Synthetic polypeptide
SYELTQPPSVSVSPGQTASITCSGDKLGDKYSFWFQQKPGQSPVLVIYHDAKRPSGIPERFSGSNSGDTATLTISGTRAMDEADYYCQAWDASTYVFGTGTKVTVL

SEQ ID NO: 95 - protein, 123 aa, Artificial Sequence
  Description of Artificial Sequence: Synthetic polypeptide
EVQLLESGGGLVQPGGSLRLSCAASGFTFSSYAMSWVRQAPGKGLEWVSAFSGWDVSTYYADAVKGRFTISRDNSKNTLYLQMNSLRAEDTAVYYCVKDGLGPYFYNYGMDVWGQGTTVTVSS

SEQ ID NO: 96 - protein, 106 aa, Artificial Sequence
  Description of Artificial Sequence: Synthetic polypeptide
SYELTQPPSVSVSPGQTASITCSGKKLGKKYSFWFQQKPGQSPVLVIYHDAKRPSGIPERFSGSNSGDTATLTISGTRAMDEADYYCQAWKASTYVFGTGTKVTVL

SEQ ID NO: 97 - protein, 123 aa, Artificial Sequence; identical to SEQ ID NO: 95
  Description of Artificial Sequence: Synthetic polypeptide

SEQ ID NO: 98 - protein, 106 aa, Artificial Sequence
  Description of Artificial Sequence: Synthetic polypeptide
SYELTQPPSVSVSPGQTASITCSGKKLGKKYSFWFQQKPGQSPVLVIYHDAKRPSGIPERFSGSNSGNTATLTISGTRAMDEADYYCQAWDASTYVFGTGTKVTVL

SEQ ID NO: 99 - protein, 123 aa, Artificial Sequence; identical to SEQ ID NO: 95
  Description of Artificial Sequence: Synthetic polypeptide

SEQ ID NO: 100 - protein, 106 aa, Artificial Sequence
  Description of Artificial Sequence: Synthetic polypeptide
SYELTQPPSVSVSPGQTASITCSGDKLGKKYSFWFQQKPGQSPVLVIYHDAKRPSGIPERFSGSNSGNTATLTISGTRAMDEADYYCQAWKASTYVFGTGTKVTVL

SEQ ID NO: 101 - protein, 123 aa, Artificial Sequence; identical to SEQ ID NO: 95
  Description of Artificial Sequence: Synthetic polypeptide

SEQ ID NO: 102 - protein, 106 aa, Artificial Sequence
  Description of Artificial Sequence: Synthetic polypeptide
SYVLTQPPSVSVSPGQTASITCSGDKLGKKYSFWFQQKPGQSPVLVIYHDAKRPSGIPERFSGSNSGDTATLTISGTRAMDEADYYCQAWKASTYVFGTGTKVTVL

SEQ ID NO: 103 - protein, 123 aa, Artificial Sequence; identical to SEQ ID NO: 95
  Description of Artificial Sequence: Synthetic polypeptide

SEQ ID NO: 104 - protein, 106 aa, Artificial Sequence
  Description of Artificial Sequence: Synthetic polypeptide
SYELTQPPSVSVSPGQTASITCSGDKLGDKYSFWFQQKPGQSPVLVIYHDAKRPSGIPERFSGSNSGDTATLTISGTRAMDEADYYCQAWDSSTYVFGTGTKVTVL

SEQ ID NO: 105 - protein, 123 aa, Artificial Sequence; identical to SEQ ID NO: 95
  Description of Artificial Sequence: Synthetic polypeptide

SEQ ID NO: 106 - protein, 17 aa, Artificial Sequence
  Description of Artificial Sequence: Synthetic peptide
AFSGSGGSTYYADSVKG

SEQ ID NO: 107 - protein, 5 aa, Artificial Sequence
  Description of Artificial Sequence: Synthetic peptide
SYAMS

SEQ ID NO: 108 - protein, 25 aa, Artificial Sequence
  Description of Artificial Sequence: Synthetic peptide
EVQLVESGGGLVKPGGSLRLSCAAS

SEQ ID NO: 109 - protein, 25 aa, Artificial Sequence
  Description of Artificial Sequence: Synthetic peptide
QVQLQESGPGLVKPSETLSLTCTVS

SEQ ID NO: 110 - protein, 25 aa, Artificial Sequence
  Description of Artificial Sequence: Synthetic peptide
EVQLLESGGGLVQPGGSLRLSCAAS

SEQ ID NO: 111 - protein, 25 aa, Artificial Sequence
  Description of Artificial Sequence: Synthetic peptide
QVQLQESGPGLVKPSETLSLTCSVS

SEQ ID NO: 112 - protein, 25 aa, Artificial Sequence
  Description of Artificial Sequence: Synthetic peptide
QVQLVESGGGVVQPGRSLRLSCAAS

SEQ ID NO: 113 - protein, 25 aa, Artificial Sequence
  Description of Artificial Sequence: Synthetic peptide
QVQLVESGGGVVQPGRSLRLSCVAS

SEQ ID NO: 114 - protein, 10 aa, Artificial Sequence
  Description of Artificial Sequence: Synthetic peptide
GFTFSSYSMN

SEQ ID NO: 115 - protein, 10 aa, Artificial Sequence
  Description of Artificial Sequence: Synthetic peptide
GGSISSYYWS

SEQ ID NO: 116 - protein, 10 aa, Artificial Sequence
  Description of Artificial Sequence: Synthetic peptide
GGSISTYYWS

SEQ ID NO: 117 - protein, 10 aa, Artificial Sequence
  Description of Artificial Sequence: Synthetic peptide
GFTFSSYAMS

SEQ ID NO: 118 - protein, 10 aa, Artificial Sequence
  Description of Artificial Sequence: Synthetic peptide
GFTFSNAWMS

SEQ ID NO: 119 - protein, 10 aa, Artificial Sequence
  Description of Artificial Sequence: Synthetic peptide
GFTFSSYGMH

SEQ ID NO: 120 - protein, 10 aa, Artificial Sequence
  Description of Artificial Sequence: Synthetic peptide
GFTFSSYDMH

SEQ ID NO: 121 - protein, 10 aa, Artificial Sequence
  Description of Artificial Sequence: Synthetic peptide
GFTFSDYNMH

SEQ ID NO: 122 - protein, 14 aa, Artificial Sequence
  Description of Artificial Sequence: Synthetic peptide
WVRQAPGKGLEWVS

SEQ ID NO: 123 - protein, 14 aa, Artificial Sequence
  Description of Artificial Sequence: Synthetic peptide
WIRQPPGKGLEWIG

SEQ ID NO: 124 - protein, 14 aa, Artificial Sequence
  Description of Artificial Sequence: Synthetic peptide
WIRQPAGKGLEWIG

SEQ ID NO: 125 - protein, 14 aa, Artificial Sequence
  Description of Artificial Sequence: Synthetic peptide
WVRQAPGKGLEWVG

SEQ ID NO: 126 - protein, 14 aa, Artificial Sequence
  Description of Artificial Sequence: Synthetic peptide
WVRQAPGKGLEWVA

SEQ ID NO: 127 - protein, 17 aa, Artificial Sequence
  Description of Artificial Sequence: Synthetic peptide
SISSSSSYIYYADSVKG

SEQ ID NO: 128 - protein, 17 aa, Artificial Sequence
  Description of Artificial Sequence: Synthetic peptide
YISTSYNYIYYADSVKG

SEQ ID NO: 129 - protein, 17 aa, Artificial Sequence
  Description of Artificial Sequence: Synthetic peptide
YISSSYNYIYYGDSVKG

SEQ ID NO: 130 - protein, 17 aa, Artificial Sequence
  Description of Artificial Sequence: Synthetic peptide
YISTSNSYIYYADSVKG

SEQ ID NO: 131 - protein, 16 aa, Artificial Sequence
  Description of Artificial Sequence: Synthetic peptide
YIYYSGSTNYNPSLKS

SEQ ID NO: 132 - protein, 17 aa, Artificial Sequence
  Description of Artificial Sequence: Synthetic peptide
AISGSGGSTYYADSVKG

SEQ ID NO: 133 - protein, 16 aa, Artificial Sequence
  Description of Artificial Sequence: Synthetic peptide
RIYTSGSTNYNPSLKS

SEQ ID NO: 134 - protein, 16 aa, Artificial Sequence
  Description of Artificial Sequence: Synthetic peptide
RIYMTGRTNYNSSLKS

SEQ ID NO: 135 - protein, 19 aa, Artificial Sequence
  Description of Artificial Sequence: Synthetic peptide
RIKSKTDGGTTDYAAPVKG

SEQ ID NO: 136 - protein, 19 aa, Artificial Sequence
  Description of Artificial Sequence: Synthetic peptide
RIRSEIDGGTTNYAAPVKG

SEQ ID NO: 137 - protein, 19 aa, Artificial Sequence
  Description of Artificial Sequence: Synthetic peptide
RIRSKIDGGTINYAAPVKG

SEQ ID NO: 138 - protein, 17 aa, Artificial Sequence
  Description of Artificial Sequence: Synthetic peptide
VIWYDGSNKYYADSVKG

SEQ ID NO: 139 - protein, 17 aa, Artificial Sequence
  Description of Artificial Sequence: Synthetic peptide
VIWYDGSNKYYADSVQG

SEQ ID NO: 140 - protein, 17 aa, Artificial Sequence
  Description of Artificial Sequence: Synthetic peptide
SISYSSTYIYYADSVRG

SEQ ID NO: 141 - protein, 32 aa, Artificial Sequence
  Description of Artificial Sequence: Synthetic polypeptide
RFTISRDNAKNSLYLQMNSLRAEDTAVYYCAR

SEQ ID NO: 142 - protein, 32 aa, Artificial Sequence
  Description of Artificial Sequence: Synthetic polypeptide
RVTISVDTSKNQFSLKLSSVTAADTAVYYCAR

SEQ ID NO: 143 - protein, 32 aa, Artificial Sequence
  Description of Artificial Sequence: Synthetic polypeptide
RFTISRDNSKNTLYLQMNSLRAEDTAVYYCAK

SEQ ID NO: 144 - protein, 32 aa, Artificial Sequence
  Description of Artificial Sequence: Synthetic polypeptide
RFTISRDNSKNTLYLQMNSLRAEDTAVYYCVQ

SEQ ID NO: 145 - protein, 32 aa, Artificial Sequence
  Description of Artificial Sequence: Synthetic polypeptide
RVTMSVDTSKNQFSLKLSSVTAADTAVYYCAR

SEQ ID NO: 146 - protein, 32 aa, Artificial Sequence
  Description of Artificial Sequence: Synthetic polypeptide
RVTMSIDTSKNQLSLKLSFMTAADTAVYYCAR

SEQ ID NO: 147 - protein, 32 aa, Artificial Sequence
  Description of Artificial Sequence: Synthetic polypeptide
RFTISRDDSKNTLYLQMNSLKTEDTAVYYCTT

SEQ ID NO: 148 - protein, 32 aa, Artificial Sequence
  Description of Artificial Sequence: Synthetic polypeptide
RFTISRDDSKNTLYLQMNSLKTEDTAVYYCAT

SEQ ID NO: 149 - protein, 32 aa, Artificial Sequence
  Description of Artificial Sequence: Synthetic polypeptide
RFTISRDDSKNTLYLQMNSLRTEDTAVYYCAT

SEQ ID NO: 150 - protein, 32 aa, Artificial Sequence
  Description of Artificial Sequence: Synthetic polypeptide
RFTISRDNSKNTLYLQMNSLRAEDTAVYYCAR

SEQ ID NO: 151 - protein, 32 aa, Artificial Sequence
  Description of Artificial Sequence: Synthetic polypeptide
RFTISRDNSKNTLYLQMNSLRAEDTAVYYCTS

SEQ ID NO: 152 - protein, 32 aa, Artificial Sequence
  Description of Artificial Sequence: Synthetic polypeptide
RFTISRDNAKNSLYLQMNSLRAEDTAVFYCAR

SEQ ID NO: 153 - protein, 12 aa, Artificial Sequence
  Description of Artificial Sequence: Synthetic peptide
DQVGATLDAFDI

SEQ ID NO: 154 - protein, 12 aa, Artificial Sequence
  Description of Artificial Sequence: Synthetic peptide
DGGHYWDDAFDI

SEQ ID NO: 155 - protein, 13 aa, Artificial Sequence
  Description of Artificial Sequence: Synthetic peptide
ADFWSGTLWGFDY

SEQ ID NO: 156 - protein, 10 aa, Artificial Sequence
  Description of Artificial Sequence: Synthetic peptide
ESGSSYSYDY

SEQ ID NO: 157 - protein, 15 aa, Artificial Sequence
  Description of Artificial Sequence: Synthetic peptide
DQVGAYYGDYYGMDV

SEQ ID NO: 158 - protein, 13 aa, Artificial Sequence
  Description of Artificial Sequence: Synthetic peptide
EDSSGWYDGWFDP

SEQ ID NO: 159 - protein, 10 aa, Artificial Sequence
  Description of Artificial Sequence: Synthetic peptide
EDYYYYGLDV

SEQ ID NO: 160 - protein, 11 aa, Artificial Sequence
  Description of Artificial Sequence: Synthetic peptide
WGQGTMVTVSS

SEQ ID NO: 161 - protein, 11 aa, Artificial Sequence
  Description of Artificial Sequence: Synthetic peptide
WGQGTTVTVSS

SEQ ID NO: 162 - protein, 11 aa, Artificial Sequence
  Description of Artificial Sequence: Synthetic peptide
WGQGTLVTVSS

SEQ ID NO: 163 - protein, 23 aa, Artificial Sequence
  Description of Artificial Sequence: Synthetic peptide
DIQMTQSPSSLSASVGDRVTITC

SEQ ID NO: 164 - protein, 23 aa, Artificial Sequence
  Description of Artificial Sequence: Synthetic peptide
DIQMTQSPSSLSASVGDRVTFTC

SEQ ID NO: 165 - protein, 11 aa, Artificial Sequence
  Description of Artificial Sequence: Synthetic peptide
RASQGIRNDLG

SEQ ID NO: 166 - protein, 11 aa, Artificial Sequence
  Description of Artificial Sequence: Synthetic peptide
RASQGIGDDLG

SEQ ID NO: 167 - protein, 11 aa, Artificial Sequence
  Description of Artificial Sequence: Synthetic peptide
RASQDITDDLG

SEQ ID NO: 168 - protein, 11 aa, Artificial Sequence
  Description of Artificial Sequence: Synthetic peptide
RASQGIRNDLD

SEQ ID NO: 169 - protein, 11 aa, Artificial Sequence
  Description of Artificial Sequence: Synthetic peptide
RASQGISNYLA

SEQ ID NO: 170 - protein, 15 aa, Artificial Sequence
  Description of Artificial Sequence: Synthetic peptide
WYQQKPGKAPKRLIY

SEQ ID NO: 171 - protein, 15 aa, Artificial Sequence
  Description of Artificial Sequence: Synthetic peptide
WYQQKPGKVPKLLIY

SEQ ID NO: 172 - protein, 15 aa, Artificial Sequence
  Description of Artificial Sequence: Synthetic peptide
WYQQKPGKVPKVLIY

SEQ ID NO: 173 - protein, 32 aa, Artificial Sequence
  Description of Artificial Sequence: Synthetic polypeptide
GVPSRFSGSGSGTEFTLTISSLQPEDFATYYC

SEQ ID NO: 174 - protein, 32 aa, Artificial Sequence
  Description of Artificial Sequence: Synthetic polypeptide
GVPPRFSGSGSGTEFTLTISSLQPEDFATYYC

SEQ ID NO: 175 - protein, 32 aa, Artificial Sequence
  Description of Artificial Sequence: Synthetic polypeptide
GVPSRFSGSGSGTDFTLTISSLQPEDVATYYC

SEQ ID NO: 176 - protein, 9 aa, Artificial Sequence
  Description of Artificial Sequence: Synthetic peptide
LQHNSYPFT

SEQ ID NO: 177 - protein, 9 aa, Artificial Sequence
  Description of Artificial Sequence: Synthetic peptide
LQHDSYPFT

SEQ ID NO: 178 - protein, 9 aa, Artificial Sequence
  Description of Artificial Sequence: Synthetic peptide
QKYNSAPFT

SEQ ID NO: 179 - protein, 9 aa, Artificial Sequence
  Description of Artificial Sequence: Synthetic peptide
LQHNSYPWT

SEQ ID NO: 180 - protein, 10 aa, Artificial Sequence
  Description of Artificial Sequence: Synthetic peptide
FGPGTKVDIK

SEQ ID NO: 181 - protein, 10 aa, Artificial Sequence
  Description of Artificial Sequence: Synthetic peptide
FGPGTRVDIK

SEQ ID NO: 182 - protein, 10 aa, Artificial Sequence
  Description of Artificial Sequence: Synthetic peptide
FGPGTKVDIR

SEQ ID NO: 183 - protein, 10 aa, Artificial Sequence
  Description of Artificial Sequence: Synthetic peptide
FGQGTKLEIK

SEQ ID NO: 184 - protein, 10 aa, Artificial Sequence
  Description of Artificial Sequence: Synthetic peptide
FGQGTKVEIK

SEQ ID NO: 185 - protein, 22 aa, Artificial Sequence
  Description of Artificial Sequence: Synthetic peptide
SYELTQPPSVSVSPGQTASITC

SEQ ID NO: 186 - protein, 22 aa, Artificial Sequence
  Description of Artificial Sequence: Synthetic peptide
SYVLTQPPSVSVAPGQTARITC

SEQ ID NO: 187 - protein, 22 aa, Artificial Sequence
  Description of Artificial Sequence: Synthetic peptide
SYELTQPPSVSVSPGQTARITC

SEQ ID NO: 188 - protein, 11 aa, Artificial Sequence
  Description of Artificial Sequence: Synthetic peptide
SGDKLGDKYAC

SEQ ID NO: 189 - protein, 11 aa, Artificial Sequence
  Description of Artificial Sequence: Synthetic peptide
SGDKLGDKYTC

SEQ ID NO: 190 - protein, 11 aa, Artificial Sequence
  Description of Artificial Sequence: Synthetic peptide
GGNNIGSKSVH

SEQ ID NO: 191 - protein, 11 aa, Artificial Sequence
  Description of Artificial Sequence: Synthetic peptide
GGNNIGSKGVH

SEQ ID NO: 192 - protein, 11 aa, Artificial Sequence
  Description of Artificial Sequence: Synthetic peptide
GGNNIGNKIVH

SEQ ID NO: 193 - protein, 11 aa, Artificial Sequence
  Description of Artificial Sequence: Synthetic peptide
SGDALPKKYAY

SEQ ID NO: 194 - protein, 11 aa, Artificial Sequence
  Description of Artificial Sequence: Synthetic peptide
SGDALPEKYAY

SEQ ID NO: 195 - protein, 15 aa, Artificial Sequence
  Description of Artificial Sequence: Synthetic peptide
WYQQKPGQSPVLVIY

SEQ ID NO: 196 - protein, 15 aa, Artificial Sequence
  Description of Artificial Sequence: Synthetic peptide
WFQQKPGQSPVLVIY

SEQ ID NO: 197 - protein, 15 aa, Artificial Sequence
  Description of Artificial Sequence: Synthetic peptide
WYQQKPGQAPVLVVY

SEQ ID NO: 198 - protein, 15 aa, Artificial Sequence
  Description of Artificial Sequence: Synthetic peptide
WYQQKSGQAPVLVIY

SEQ ID NO: 199 - protein, 7 aa, Artificial Sequence
  Description of Artificial Sequence: Synthetic peptide
QDSKRPS

SEQ ID NO: 200 - protein, 7 aa, Artificial Sequence
  Description of Artificial Sequence: Synthetic peptide
DDSDRPS

SEQ ID NO: 201 - protein, 7 aa, Artificial Sequence
  Description of Artificial Sequence: Synthetic peptide
EDSKRPS

SEQ ID NO: 202 - protein, 7 aa, Artificial Sequence
  Description of Artificial Sequence: Synthetic peptide
EDTKRPS

SEQ ID NO: 203 - protein, 32 aa, Artificial Sequence
  Description of Artificial Sequence: Synthetic polypeptide
GIPERFSGSNSGNTATLTISGTQAMDEADYYC

SEQ ID NO: 204 - protein, 32 aa, Artificial Sequence
  Description of Artificial Sequence: Synthetic polypeptide
GIPERFSGSNSGDTATLTISGTQAMDEADYYC

SEQ ID NO: 205 - protein, 32 aa, Artificial Sequence
  Description of Artificial Sequence: Synthetic polypeptide
GIPERFSGSNSGNTATLTISRVEAGDEADYYC

SEQ ID NO: 206 - protein, 32 aa, Artificial Sequence
  Description of Artificial Sequence: Synthetic polypeptide
GIPERFSGSSSGTMATLTISGAQVEDEADYYC

SEQ ID NO: 207 - protein, 12 aa, Artificial Sequence
  Description of Artificial Sequence: Synthetic peptide
QVWVSSSDHHVV

SEQ ID NO: 208 - protein, 11 aa, Artificial Sequence
  Description of Artificial Sequence: Synthetic peptide
QVWDSSSDHVV

SEQ ID NO: 209 - protein, 11 aa, Artificial Sequence
  Description of Artificial Sequence: Synthetic peptide
HSTDSSGNHGV

SEQ ID NO: 210 - protein, 11 aa, Artificial Sequence
  Description of Artificial Sequence: Synthetic peptide
YSTDSSGNHGV

SEQ ID NO: 211 - protein, 10 aa, Artificial Sequence
  Description of Artificial Sequence: Synthetic peptide
FGTGTKVTVL

SEQ ID NO: 212 - protein, 10 aa, Artificial Sequence
  Description of Artificial Sequence: Synthetic peptide
FGGGTKLTVL

SEQ ID NO: 213 - protein, 10 aa, Artificial Sequence
  Description of Artificial Sequence: Synthetic peptide
FGGGTKLTVV

SEQ ID NO: 214 - protein, 113 aa, Artificial Sequence
  Description of Artificial Sequence: Synthetic polypeptide
MGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 215 - protein, 49 aa, Artificial Sequence
  Description of Artificial Sequence: Synthetic polypeptide
CPAPELLGGPSVFLFPPKPKDTLYITREPEVTCVVVDVSHEDPEVKFNW

SEQ ID NO: 216 - protein, 49 aa, Artificial Sequence
  Description of Artificial Sequence: Synthetic polypeptide
CPAPELLGGPSVFLFPPKPKDTLMISRTPEVTCVVVDVSHEDPEVKFNW

SEQ ID NO: 217 - protein, 11 aa, Artificial Sequence
  Description of Artificial Sequence: Synthetic peptide
SGDKLGDKYAF

SEQ ID NO: 218 - protein, 9 aa, Artificial Sequence
  Description of Artificial Sequence: Synthetic peptide
QAWKASTYV

SEQ ID NO: 219 - protein, 7 aa, Artificial Sequence
  Description of Artificial Sequence: Synthetic peptide
AASSLQS

SEQ ID NO: 220 - protein, 7 aa, Artificial Sequence
  Description of Artificial Sequence: Synthetic peptide
DASSLQS

SEQ ID NO: 221 - protein, 7 aa, Artificial Sequence
  Description of Artificial Sequence: Synthetic peptide
AASTLQS

SEQ ID NO: 222 - protein, 12 aa, Unknown
  Description of Unknown: Phage-display derived peptide sequence
YKIGEPHKDTYM

SEQ ID NO: 223 - protein, 51 aa, Homo sapiens
TQRMLSGFCPHKVSAGQFSSLHVRDTKIEVAQFVKDLLLHLKKLFREGRFN